(19) United States
(12) Patent Application Publication
(10) Pub. No.: US 2025/0270278 A1
(43) Pub. Date: Aug. 28, 2025

(54) METHODS OF MAKING WATER-SOLUBLE PROTEIN FORMED IN A BACTERIAL EXPRESSION SYSTEM, COMPOSITIONS, AND METHODS OF USE THEREOF

(71) Applicant: THE RESEARCH FOUNDATION FOR THE STATE UNIVERSITY OF NEW YORK, Albany, NY (US)

(72) Inventors: Allen Norin, New York, NY (US); Sheerin Alandejani, Brooklyn, NY (US)

(21) Appl. No.: 18/265,883

(22) PCT Filed: Dec. 7, 2021
(86) PCT No.: PCT/US2021/062280

§ 371 Date: Jun. 7, 2023

Related U.S. Application Data
(60) Provisional application No. 63/281,132, filed on Nov. 19, 2021.
(60) Provisional application No. 63/122,451, filed on Dec. 7, 2020.

(51) Int. Cl. C07K 14/705; C07K 1/107; C07K 1/14

(52) U.S. Cl. CPC C07K 14/705; C07K 1/1077; C07K 1/145; C07K 2319/21

(57) ABSTRACT

The present disclosure relates to a polypeptide which is a variant of SEQ ID NO:1, fragment thereof, or fusion polypeptide, including: the variant of SEQ ID NO:1 or a fragment thereof, having an increased water solubility compared to SEQ ID NO:1 which binds to moesin. In embodiments, the polypeptide includes an affinity tag. Compositions and methods of detecting naïve NK cells using one or more moesin binding partners are also disclosed.

### CROSS-REFERENCE TO RELATED APPLICATIONS

The present disclosure claims priority or the benefit under 35 U.S.C. § 119 of U.S. provisional application No. 63/122,451 filed Dec. 7, 2020, and U.S. Provisional application No. 63/281,132 filed Nov. 19, 2021, both of which are herein entirely incorporated by reference.

### REFERENCE TO A SEQUENCE LISTING

This application contains a Sequence Listing in computer readable form, which is incorporated herein by reference.

### FIELD OF THE INVENTION

This disclosure relates to the formation of one or more water-soluble peptides or proteins in a bacterial expression system, as well as compositions, and down-stream assays in aqueous solutions using one or more water-soluble peptides or proteins formed in a bacterial expression system. In some embodiments, the present disclosure relates to a method of detecting, counting, or isolating naïve natural killer cells (nNK cells) and separating them from T and B cells using compositions and methods of the present disclosure.

### BACKGROUND

Tumor cells may be recognized and killed by activated natural killer (NK) cells or in certain cases, by naïve NK cells. Naïve NK (nNK) cells (CD3⁻, CD16⁺, TCR⁻), unlike cytolytic T lymphocytes (CTL) (CD3⁺, CD16⁻, TCR⁺), provide cell-mediated lytic activity against virus infected cells and certain tumors without requirement for activation. Such unactivated lymphocytes, also referred to as resting or naïve NK cells, are capable of destroying a narrow spectrum of tumor cells. See Trinchieri, G. in Adv Immunol 47:187-376, (1989); Whiteside T. L., in Current Protocols in Immunology, J. E. Coligan et al. eds., Supplement 17, Unit 7-18, John Wiley & Sons, New York (1996). Upon activation with lymphokines such as IL-2, NK cells acquire broad anti-tumor lytic activity, becoming lymphokine activated killer cells (LAK cells). The mechanism(s) by which naïve NK cells recognize their target cells is not completely understood. Interaction of cellular adhesion molecules and recognition of specific target structure(s) have been proposed as critical initial events in the cell mediated lytic process. Trinchieri, G. in Adv Immunol 47:187-376, (1989); Storkus, W. J. and J. R. Dawson Critical Rev. Immunol. 10:393-416 (1989).

A surface protein, p38.5, on the prototypical nNK cell target, K562 was previously identified that serves as a cognate receptor for cytotoxicity. See B. Das, M. O. Mondragon, S. Z. Tao, A. J. Norin, J Exp Med 185, 1735-1742 (1997). This protein was named “Haymaker” (HYMKR) after the complete amino acid sequence was determined (GenBank accession no. AF316402). See Genetic Identity and differential expression of p38.5 (Haymaker) in human malignant and nonmalignant cells by B. Das, S. Z. Tao, R. Mushnitsky, A. J. Norin, Int J Cancer 94, 800-806 (2001).

The amino acid sequence of HYMKR is identical to the human homologue of the translocase of outer mitochondrial membrane (TOMM40) (GenBank accession no. AF043250). The protein may be referred to as HYMKR when it localizes to the leukemia cell surface, and TOMM40 when it localizes to the mitochondria. In certain leukemia cells (K562, Jurkat, and Molt-4), HYMKR is found on the cell surface as well as in the outer membrane of mitochondria (i.e. as TOMM40). See B. Das, M. O. Mondragon, S. Z. Tao, A. J. Norin, J Exp Med 185, 1735-1742 (1997) and B. Das, S. Z. Tao, R. Mushnitsky, A. J. Norin, Int J Cancer 94, 800-806 (2001).

In normal cells and epithelial-derived cancers, HYMKR does not localize to the plasma membrane. See B. Das, M. O. Mondragon, S. Z. Tao, A. J. Norin, J Exp Med 185, 1735-1742 (1997) and B. Das, S. Z. Tao, R. Mushnitsky, A. J. Norin, Int J Cancer 94, 800-806 (2001). In mitochondria, TOMM40 forms a pore and together with other proteins facilitates protein transport into this organelle from the cytosol. See D. Rapaport, W. Neupert, J Cell Biol 146, 321-331 (1999); T. Becker et al., J Mol Biol 405, 113-124 (2011); and N. Bolender, A. Sickmann, R. Wagner, C. Meisinger, N. Pfanner, EMBO Rep 9, 42-49 (2008). HYMKR's function at the leukemia cell surface is unknown, but it may also form a pore in the plasma membrane. See U. Ahting et al., J Cell Biol 153, 1151-1160 (2001); and K. Tucker, E. Park, Nat Struct Mol Biol 26, 1158-1166 (2019).

Prior art of interest includes U.S. Application Publication No. US20030040029A1 to inventor AJ Norin entitled Detection of tumor marker transcript and protein recognized by naïve natural killer cells published Feb. 27 2003 (herein entirely incorporated by reference) relating to, inter alia, methods for detection, diagnosis and monitoring of tumor cells in a sample of mammalian cells.

To date, the nNK receptor that targets K562 cells for destruction via interaction with HYMKR is heretofore undisclosed. Moreover, the inventors have observed that methods for forming recombinant proteins or polypeptides in bacterial expression systems, including those used to investigate nNK receptors, are deficient and problematically limited by the formation of cytoplasmic aggregates (inclusion bodies or “IBs”), that are water insoluble polypeptides. The inventors have observed that the water insoluble polypeptides so synthesized in bacterial expression systems form precipitates that are functionally inactive in aqueous solutions. While formation of functionally active recombinant proteins may be possible in eukaryotic expression systems, the eukaryotic systems are more complicated and may have lower yields and certain other drawbacks compared to bacterial systems.

What are needed are methods of detecting naïve NK cells, probes, and compositions suitable for detecting and/or isolating naïve NK cells, as well as bacterial expression systems with high level expression and/or recovery of water soluble recombinant proteins or polypeptides-of-interest.

### SUMMARY

In embodiments, the present disclosure relates to a polypeptide which is a variant of SEQ ID NO:1, a fragment thereof, or fusion polypeptide, including: a variant of SEQ ID NO:1 or a fragment thereof having an increased water solubility compared to SEQ ID NO:1 which binds to moesin, wherein the polypeptide includes an affinity tag. In some embodiments, the affinity tag is a polyhistidine tag or a hexahistidine tag.

In some embodiments, the present disclosure relates to a variant of a parent HYMKR polypeptide including: a plurality of deletions of amino acid residues corresponding to amino acid residues M1 to P78 and D198 to G361 using SEQ ID NO:1 for numbering, wherein the variant includes biotin and increased water-solubility compared to the parent HYMKR polypeptide.

In some embodiments, the present disclosure relates to a method for preparing a polypeptide, fragment, or fusion polypeptide or fragment thereof, including: a) contacting a polypeptide, fragment, variant, or fusion polypeptide precipitate obtained from bacteria cells with an aqueous urea solution in an amount sufficient to solubilize the precipitate and form a dissolved polypeptide, fragment, variant, or fusion polypeptide within an aqueous medium; b) contacting the dissolved polypeptide, fragment, variant, or fusion polypeptide within the aqueous medium with biotin under conditions sufficient to bind biotin to the dissolved polypeptide, fragment, variant, or fusion polypeptide and form biotinylated dissolved polypeptide, fragment, variant, or fusion polypeptide; and c) recovering the biotinylated dissolved polypeptide, fragment, variant, or fusion polypeptide as a water-soluble biotinylated polypeptide, fragment, variant, or fusion polypeptide.

In some embodiments, the present disclosure relates to a method for producing a water-soluble polypeptide in a bacterial host cell, the method including: transforming one or more bacterial host cells with an expression vector including a nucleotide sequence encoding a moesin binding partner; culturing the transformed bacterial host is cells in a medium suitable for expression of the moesin binding partner, and dissolving the moesin binding partner in an aqueous urea mixture including a detectable label moiety to form a water-soluble labelled moesin binding partner.

In some embodiments, the present disclosure relates to a synthetic biotinylated variant probe, including an amino acid sequence including a moesin binding partner, wherein the moesin binding partner includes a detectable label moiety.

In some embodiments, the present disclosure relates to a method for determining a number of naïve natural killer (NK) cells in a biological sample, including: (a) contacting a sample containing naïve NK cells with one or more moesin binding partners including a detectable label moiety and having specific binding affinity for moesin, under conditions permissive for binding of the one or more moesin binding partners expressed by naïve NK cells in the sample; and (b) counting the number of cells bound by the moesin binding partners to determine the number of naïve natural killer cells in the sample.

In embodiments, the present disclosure relates to a method for identifying or determining a number of naïve natural killer (NK) cells in a biological sample, including: (a) contacting a sample containing naïve NK cells with one or more moesin binding partners including a detectable label moiety and having specific binding affinity for moesin, under conditions permissive for binding of the one or more moesin binding partners expressed by naïve NK cells in the sample, wherein the one or more moesin binding partners are an antibody with a binding affinity to moesin, or a synthetic biotinylated variant probe configured to bind moesin; and (b) detecting the moesin binding partners to identify or determine the number of naïve natural killer cells in the sample.

In some embodiments, the present disclosure relates to a method of identifying cytotoxic naïve natural killer (NK) cells, including: (a) contacting a sample containing naïve NK cells with one or more moesin binding partners including a detectable label moiety and having specific binding affinity for moesin, under conditions permissive for binding of the one or more moesin binding partners expressed by naïve NK cells in the sample, wherein the one or more moesin binding partners are an antibody with a binding affinity to moesin, or a synthetic biotinylated variant probe configured to bind moesin; and (b) detecting the moesin binding partners to identify or determine the presence of cytotoxic naïve natural killer cells.

In some embodiments, the present disclosure relates to a method of isolating one or more naïve NK cells from a blood sample, including: contacting one or more moesin binding partners with one or more magnetic beads under conditions sufficient to form one or more conjugated magnetic beads; contacting the one or more conjugated magnetic beads with a blood sample under conditions sufficient to form one or more magnetic bead/naïve NK cell complexes; and separating the one or more magnetic bead/naïve NK cell complexes to isolate the naïve NK cells from the blood sample.

In some embodiments, the present disclosure relates to a method of isolating one or more naïve NK cells from a blood sample, including: contacting one or more moesin binding partners with one or more substrates under conditions sufficient to form one or more conjugated substrates; contacting the one or more conjugated substrates with a blood sample under conditions sufficient to form one or more substrate/naïve NK cell complexes; and separating the one or more substrate/naïve NK cell complexes to isolate the naïve NK cells from the blood sample.

In some embodiments, the present disclosure relates to a bead including: a substrate; and one or more moesin binding partners.

In some embodiments, the present disclosure relates to a method of selecting T and B cells, from a mixture of cells, including: contacting a mixture of cells with the bead of the present disclosure under conditions that bind the bead to one or more nNK cells within the mixture of cells, if any, within a container; immobilizing the bead within the container; and eluting T and B cells from the mixture of cells, if any.

The illustrative aspects of the present disclosure are designed to solve the problems herein described and/or other problems not discussed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. These and other features of this disclosure will be more readily understood from the following detailed description of the various aspects of the disclosure taken in conjunction with the accompanying drawings that depict various embodiments of the disclosure, in which:

FIG. 1 depicts a Coomassie blue stained SDS-PAGE of uninduced and induced recombinant HYMKR polypeptide (HYMKR⁷⁹⁻¹⁹⁷).

FIGS. 2A, 2B, and 2C each independently depict a plurality of panels relating to biotinylated-HYMKR⁷⁹⁻¹⁹⁷ polypeptide interacting strongly with nNK cells compared to T cells. Flow cytometry data is shown to examine the binding of biotinylated-HYMKR⁷⁹⁻¹⁹⁷ to CD3⁺ T cells and CD16⁺ NK cells of three healthy subjects.

FIG. 2D depicts data relating to the binding of biotinylated-HYMKR⁷⁹⁻¹⁹⁷ to CD3⁺ T cells and CD16⁺ NK cells of three healthy subjects, Donor 1, Donor 2, and Donor 3.

FIGS. 3A and 3B depicts data relating to affinity chromatography purification of surface biotinylated proteins of NK cells from HYMKR⁷⁹⁻¹⁹⁷-Ni²⁺ resin.

FIG. 4 depicts data relating to experimental approaches to identify the plasma membrane surface protein on nNK cells that binds to the HYMKR⁷⁹⁻¹⁹⁷-Ni²⁺ resin.

FIG. 5 depicts data relating to the mono-specificity of affinity purified rabbit anti-moesin antibody.

FIGS. 6A, 6B, and 6C each independently depict a plurality of panels relating to reactivity with anti-moesin antibody indicating cell surface expression on nNK cells. Flow cytometry experiments were performed to determine if moesin was expressed on the surface of different lymphocyte subpopulations. Affinity purified rabbit anti-moesin antibody against amino acids 1 to 294 was used (0.3 μg) as described in Materials and Methods

FIG. 6D depicts a table with data indicating cell surface expression of moesin on nNK cells was significantly greater than on T cells (94-fold, p=0.0087).

FIGS. 6E, 6F, and 6G depict data showing three cell types expressed the molecule intracellularly at similar levels in permeabilized lymphocytes.

FIG. 7 depicts immunological identity of the 70 kDa lymphocyte protein purified by affinity chromatography on a HYMKR⁷⁹⁻¹⁹⁷-Ni-resin.

FIGS. 8A and 8B depict models of surface localization of moesin in NK cells: interaction with radixin in nNK cells.

FIGS. 9A and 9B depict, respectively, the nucleotide and amino acid sequences of the HYMKR construct of the present disclosure.

FIGS. 10A, 10B and 10C each independently depict a plurality of panels showing the reactivity of CD19⁺ B lymphocytes of three subjects with affinity purified anti-moesin antibody.

FIG. 11 depicts a composition embodiment of the present disclosure.

FIG. 12 depicts a process flow of embodiments of the present disclosure.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

The inventors have found that the HYMKR (Haymaker) polypeptide, characteristically expressed on the surface of certain tumor cells susceptible to cell-mediated lysis by naïve (non-activated) human natural killer (NK) cells, binds to moesin disposed atop the surface of naïve NK cells. The inventors have surprisingly found moesin on the surface of nNK cells, but not on T or B cells. With the understanding that HYMKR binds to moesin, embodiments of the present disclosure provide methods of detecting naïve NK cells, probes, and compositions suitable for binding to moesin and/or identifying, detecting, or isolating naïve NK cells. Further, the inventors have surprisingly found water insoluble proteins, including water-insoluble polypeptides generated from bacterial expression systems, may be altered to become or remain water-soluble and/or biotinylated using methods of the present disclosure. For example, a method of producing water soluble polypeptides that are normally insoluble in bacterial expression systems is provided. Accordingly, the methods and compositions of the present disclosure advantageously provide high level expression and/or recovery of one or more water-soluble proteins or polypeptides formed from a bacterial expression system, as well as compositions, and down-stream assays in aqueous solutions using the one or more water-soluble proteins or polypeptides formed in a bacterial expression system initially in water-insoluble form. In embodiments, the methods of the present disclosure may advantageously save time and effort to produce functionally active recombinant polypeptides. In some embodiments, a robust method of detecting, counting, or isolating naïve natural killer cells (nNK cells) is also provided. In embodiments, water-soluble polypeptides of the present disclosure may be used in methods of selective detection, identification, and separation of nNK cells and/or NK cells in biological samples, such as blood samples.

### Definitions

As used in the present specification, the following words and phrases are generally intended to have the meanings as set forth below, except to the extent that the context in which they are used indicates otherwise.

As used herein, the singular forms “a” “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [Cl 95%] for the mean) or within ±10% of the indicated value, whichever is greater.

cDNA: The term “cDNA” means a DNA molecule that can be prepared by reverse transcription from a mature, spliced, mRNA molecule obtained from a eukaryotic or prokaryotic cell. cDNA lacks introns or intron sequences that may be present in corresponding genomic DNA. In embodiments, cDNA may refer to a nucleotide sequence that correspond to the nucleotide sequence of an mRNA from which it is derived.

Coding sequence: The term “coding sequence” means a polynucleotide, which directly specifies the amino acid sequence of a polypeptide. The boundaries of the coding sequence are generally determined by an open reading frame, which begins with a start codon and ends with a stop codon. The coding sequence may be a genomic DNA, cDNA, synthetic DNA, or a combination thereof.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Non-limiting exemplary conservative amino acid substitution groups are: valine-leucine-isoleucine, phenylalanine-tyrosine, lysine-arginine, alanine-valine-glycine, and asparagine-glutamine.

As used herein, the term “forming a mixture” refers to the process of bringing into contact at least two distinct species such that they mix together and interact. “Forming a reaction mixture” and “contacting” refer to the process of bringing into contact at least two distinct species such that they mix together and can react, either modifying one of the initial reactants or forming a third, distinct, species, a product. It should be appreciated, however, the resulting reaction product can be produced directly from a reaction between the added reagents or from an intermediate from one or more of the added reagents which can be produced in the reaction mixture. “Conversion” and “converting” refer to a process including one or more steps wherein a species is transformed into a distinct product.

The terms “deoxyribonucleotide” and “DNA” refer to a nucleotide or polynucleotide including at least one ribosyl moiety that has an H at the 2′ position of a ribosyl moiety. In embodiments, a deoxyribonucleotide is a nucleotide having an H at its 2′ position.

As used herein the term “fragment” means a polypeptide having one or more amino acids absent from the amino and/or carboxyl terminus of a mature polypeptide or domain, wherein the fragment is able to bind to a moesin. In one aspect, a fragment contains at least 1% to 75%, at least 2% to 40% or about 2 to 30% of the number of amino acids of the mature polypeptide of SEQ ID NO: 1. In another aspect, a fragment contains at least 70% to 99%, at least 90% to 99% or about 95 to 99% of the number of amino acids of the mature polypeptide of SEQ ID NOS: 2-14.

The terms “host cell” or “host microorganism” refer to a microorganism capable of receiving foreign or heterologous genes and of expressing those genes to produce an active gene product.

The term “isolated” means a substance in a form or environment that does not occur in nature. For example, an isolated polypeptide may include a polypeptide or a fragment, variant, or derivative thereof that is not in its natural milieu. No particular level of purification is required. For example, an isolated polypeptide can be removed from its native or natural environment. Recombinantly produced polypeptides and proteins expressed in host cells are considered isolated for purposes of the present disclosure, as are native or recombinant polypeptides which have been separated, fractionated, or partially or substantially purified by any suitable technique.

The term “mature polypeptide” means a polypeptide in its final form following translation and any post-translational modifications.

The term “natural killer (NK) cells” as used herein, refers to non-T, non-B lymphocytes. In embodiments, “natural killer (NK) cells” as used herein may refer to non-T, non-B lymphocytes including one or more of the following properties: they have spontaneous lytic activity against cells infected with intra-cellular parasites (e.g. viruses) and certain types of tumor cells (usually of hematologic origin). They express various combinations of CD8, CD16 and CD56 on their surface and lack the surface expression of CD3 components, TCR heterodimers and immunoglobulins (e.g., IgM, IgD). They express cell surface molecules that regulate cytolytic activity by interaction with MHC class-1 molecules. They may be stimulated by incubation with cytokines, e.g. IL-2, to express lytic activity against a broader spectrum of target cells. In embodiments, NK cells that have not been stimulated/activated with cytokines in vivo or in vitro are termed naïve or non-activated NK cells.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. A polypeptide may be derived from a natural biological source or produced by recombinant technology, but is not necessarily translated from a designated nucleic acid sequence. It may be generated in any manner including by chemical synthesis.

The terms “sequence identity”, “identity” and the like as used herein with respect to polynucleotide or polypeptide sequences refer to the nucleic acid residues or amino acid residues in two sequences that are the same when aligned for maximum correspondence over a specified comparison window. Thus, “percentage of sequence identity”, “percent identity” and the like refer to the value determined by comparing two optimally aligned sequences over a comparison window, wherein the portion of the polynucleotide or polypeptide sequence in the comparison window may include additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity.

Percent identity can be readily determined by any known method, including but not limited to those described in: 1) Computational Molecular Biology (Lesk, A. M., Ed.) Oxford University: NY (1988); 2) Biocomputing: Informatics and Genome Projects (Smith, D. W., Ed.) Academic: NY (1993); 3) Computer Analysis of Sequence Data, Part I (Griffin, A. M., and Griffin, H. G., Eds.) Humana: NJ (1994); 4) Sequence Analysis in Molecular Biology (von Heinje, G., Ed.) Academic (1987); and 5) Sequence Analysis Primer (Gribskov, M. and Devereux, J., Eds.) Stockton: NY (1991), all of which are incorporated herein by reference. In embodiments, sequence identity between two amino acid sequences is determined using the Needleman-Wunsch algorithm (Needleman and Wunsch, 1970, J. Mol. Biol. 48: 443-453). In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety.

The term “recombinant” when used herein to characterize a DNA sequence such as a plasmid, vector, or construct refers to an artificial combination of two otherwise separated segments of sequence, e.g., by chemical synthesis and/or by manipulation of isolated segments of nucleic acids by genetic engineering techniques.

As used herein, “moesin binding partner” refers to a compound that selectively binds to any portion of moesin. In embodiments, a moesin binding partner is a selective binding compound. In embodiments, a moesin binding partner may be an antibody that binds to moesin. In embodiments, a moesin binding partner may be a protein or polypeptide that binds to moesin.

The term “recovering” refers to separating a chemical compound from an initial mixture to obtain the compound in greater purity or at a higher concentration than the purity or concentration of the compound in the initial mixture.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

As used herein, the term “target protein” refers to a molecule or a portion of a protein capable of being bound by a selective binding compound. In certain embodiments, a target protein is moesin or portions of moesin.

Variant: The term “variant” means a polypeptide such as a moesin binding partner including an alteration, i.e., a substitution, insertion, and/or deletion, at one or more positions. A substitution means replacement of the amino acid occupying a position with a different amino acid; a deletion means removal of the amino acid occupying a position; and an insertion means adding one or more (e.g., several) amino acids, e.g., 1-10 amino acids, adjacent to the amino acid occupying a position.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Certain abbreviations are used herein and have the following meanings:
DPBS, dulbecco's phosphate-buffered saline;
ERM, ezrin, radixin, and moesin;
FBS, fetal bovine serum;
HBSS, hank's balanced salt solution;
HLA, human leukocyte antigens,
HYMKR, Haymaker (also unknown as TOMM40);
HYMKR⁷⁹⁻¹⁹⁷-Ni²⁺ resin, HYMKR⁷⁹⁻¹⁹⁷ bound to Ni²⁺ resin;
K562, human erythroleukemic cell line;
IL-2, Interleukin-2;
Kan, kanamycin.

### DESCRIPTION OF CERTAIN EMBODIMENTS

Embodiments of the present disclosure provide methods of detecting naïve NK cells, probes, compositions suitable for binding to moesin or portions thereof, and/or methods of purifying or identifying naïve NK cells such as cytotoxic naïve NK cells. Without being bound by theory, the role played by moesin in binding to the HYMKR polypeptide, suggests that one or more moesin binding partners are useful in the detection of nNK cells. Further, water-soluble proteins, including water-soluble polypeptides generated from bacterial expression systems, may be generated and/or biotinylated using methods of the present disclosure, and/or are suitable for binding to moesin.

In some embodiments, a moesin binding partner is characterized as polypeptide configured to bind to moesin (SEQ ID NO:15) or to a segment thereof. In some embodiments, a moesin binding partner of the present disclosure includes or consists of one or more isolated polypeptides, or polypeptides having a sequence identity to the mature polypeptide of SEQ ID NO: 1 of at least 60%, at least 70%, at least 80%, e.g., at least 85%, at least 90%, at least 91%, at least 92%, at least 93%, at least 94%, at least 95%, at least 96%, at least 97%, at least 98%, at least 99%, or 100%, having the ability to bind to moesin or a segment thereof. In some embodiments, a moesin binding partner of the present disclosure includes or consists of one or more polypeptides having a sequence identity to the mature polypeptide of SEQ ID NO: 2 of at least 60%, at least 70% e.g., at least 85%, at least 90%, at least 91%, at least 92%, at least 93%, at least 94%, at least 95%, at least 96%, at least 97%, at least 98%, at least 99%, or 100%, having the ability to bind to moesin or a segment thereof. In some embodiments, each polypeptide is soluble in water at 25 degrees Celsius. In embodiments, the moesin binding partner is a polypeptide characterized as isolated, or substantially purified.

In some embodiments, a moesin binding partner of the present disclosure includes or consists of one or more polypeptides having identical subsequences to the corresponding subsequences in the 79-197 segment of SEQ ID NO: 1.

In embodiments, the present disclosure relates to a polypeptide which is a variant of SEQ ID NO:1, a fragment thereof, or fusion polypeptide, including: a variant of SEQ ID NO:1 or a fragment thereof having an increased water solubility compared to SEQ ID NO:1 which binds to moesin.

In embodiments, polypeptides of the present disclosure include or consist of one or more polypeptides including residues corresponding to 79-197 segment of SEQ ID NO: 1, and further include (His)ₙ added to the amino terminal end, or carboxyl terminal end, wherein n is a number from 1 to 8. In embodiments, polypeptides of the present disclosure comprise or consist of one or more polypeptides including residues corresponding to 79-197 segment of SEQ ID NO: 1, and further include (His)ₙ added to the carboxyl terminal end, wherein n is a number from 1 to 11, or wherein n is a number from 2 to 11, or wherein n=6. In embodiments, polypeptides of the present disclosure include or consist of one or more synthetic polypeptides including residues of SEQ ID NO: 2, and further include (His)ₙ added to the carboxyl terminal end, wherein n is a number from 1 to 11, or wherein n is a number from 2 to 11, or wherein n is a number from 2-8. In embodiments, polypeptides of the present disclosure comprise or consist of one or more synthetic polypeptides including residues of one of SEQ ID NOS. 2, and further including (Xaa)ₙ added to the carboxyl terminal end, wherein in Xaa is selected from the group consisting of arginine, lysine, histidine, and combinations thereof, wherein n is a number from 1 to 11, or wherein n is a number from 2 to 11, or wherein n is a number 2-8. In some embodiments, n=0. In some embodiments, Xaa may refer to any amino acid. However, in some embodiments, Xaa is used to identify a group of selected amino acids. For example, in embodiments, Xaa is selected from the group consisting of arginine, lysine, histidine, and combinations thereof. In embodiments, Xaa is selected from the group consisting of arginine, lysine, and combinations thereof. In embodiments, Xaa is selected from the group consisting of arginine, histidine, and combinations thereof. In embodiments, Xaa is selected from the group consisting of arginine, and histidine. In embodiments, Xaa is selected from the group consisting of lysine, histidine, and combinations thereof. In some embodiments, such as where more than one Xaa is provided, two or more Xaa's may be the same or different.

In embodiments, the moesin binding partners of the present disclosure have the ability to bind to moesin or a segment thereof disposed atop the top surface of an nNK cell. In some embodiments, each polypeptide or moesin binding partner is soluble in water at 25 degrees Celsius. In embodiments, the polypeptide or moesin binding partner is characterized as isolated, or substantially purified.

In some embodiments, a moesin binding partner of the present disclosure includes or consists of a polypeptide having a sequence identity to the polypeptide or mature polypeptide of SEQ ID NOS: 2-14 of at least 80%, e.g., at least 85%, at least 90%, at least 91%, at least 92%, at least 93%, at least 94%, at least 95%, at least 96%, at least 97%, at least 98%, at least 99%, or 100%, which have the ability to bind to moesin. In embodiments, the polypeptide is characterized as isolated, and/or substantially purified.

In some embodiments, moesin binding partner of the present disclosure includes one or more polypeptides having a sequence identity to the mature polypeptide of SEQ ID NOS: 3-8 of at least 80%, e.g., at least 85%, at least 90%, at least 91%, at least 92%, at least 93%, at least 94%, at least 95%, at least 96%, at least 97%, at least 98%, at least 99%, or 100%, which have the ability to bind to moesin. In embodiments, X or Xaa is selected from the group consisting of arginine (R), Histidine (H), Lysine (K), and combinations thereof. In embodiments, X or Xaa is Histidine (H), or each X or Xaa is Histidine (H). In embodiments, the polypeptide is characterized as isolated, or substantially purified.

In some embodiments, a moesin binding partner of the present disclosure includes or consists of a polypeptide having the amino acid sequence consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14, having the ability to bind to moesin. In embodiments, X or Xaa is selected from the group consisting of Arginine (R), Histidine (H), Lysine (K), and combinations thereof. In some embodiments, each polypeptide is soluble in water at 25 degrees Celsius. In embodiments, the polypeptide is characterized as isolated, or substantially purified. in embodiments, Xaa is selected from the group consisting of arginine, lysine, histidine, and combinations thereof. In embodiments, Xaa is selected from the group consisting of arginine, and lysine. In embodiments, Xaa is selected from the group consisting of arginine, histidine, and combinations thereof. In embodiments, Xaa is selected from the group consisting of arginine, and histidine. In embodiments, Xaa is selected from the group consisting of lysine, histidine, and combinations thereof. In some embodiments, such as where more than one Xaa is provided, two or more Xaa's may be the same or different.

In some embodiments, moesin binding partner of the present disclosure includes a plurality of polypeptides having amino acid sequences selected from the group consisting of: SEQ ID NO: 2, SEQ ID NO: 3, SEQ ID NO: 4. SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and combinations thereof. In embodiments, each amino acid sequence has the ability to bind to moesin or a segment thereof. In embodiments, X or Xaa is selected from the group consisting of Arginine (R), Histidine (H), Lysine (K), and combinations thereof. In some embodiments, each polypeptide is soluble in water at 25 degrees Celsius. In embodiments, the polypeptide is characterized as isolated, or substantially purified.

In some embodiments, suitable moesin binding partner may include a polypeptide which is a variant of SEQ ID NO:1, fragment thereof or fusion polypeptide, including: a variant of SEQ ID NO:1 or a fragment thereof having an increased water solubility compared to SEQ ID NO:1 and binds moesin, wherein the polypeptide includes an affinity tag. In some embodiments, the affinity tag is bound to a carboxy terminal end of the polypeptide. In some embodiments, the polypeptide includes a plurality of deletions of amino acid residues corresponding to amino acid residues M1 to P78, D198 to G361, or M1 to P78 and D198 to G361 using SEQ ID NO:1 for numbering. In some embodiments, the polypeptide includes biotin. In some embodiments, the sequence identity of the polypeptide to SEQ ID NO: 1 or SEQ ID NO: 2 is more than 90%, more than 95%, more than 96%, more than 97%, more than 98%, or more than 99%. In embodiments, the polypeptide is characterized as isolated, or substantially purified.

In some embodiments, suitable moesin binding partner may include a variant of a parent HYMKR polypeptide including: a plurality of deletions of amino acid residues corresponding to amino acid residues M1 to P78 and D198 to G361 using SEQ ID NO:1 for numbering, wherein the variant includes biotin and increased water-solubility compared to the parent HYMKR polypeptide. In some embodiments, the moesin binding partner may include an affinity tag. In embodiments, the affinity tag includes one or more positive electrically charged side chains including arginine, histidine, lysine, or combinations thereof. In some embodiments, the affinity tag includes at least two histidine residues. In some embodiments, the variant further includes one or more additions of histidine amino acid residues corresponding to amino acid residues D198, D198 to F199, D198 to T200, D198 to A201, D198 to A202, or D198 to V203, using SEQ ID NO:1 for numbering, and wherein the variant has increased water solubility and moesin protein binding activity compared to the parent haymaker polypeptide.

In embodiments, the parent HYMKR polypeptide is from Homo sapiens. In some embodiments, the parent HYMKR polypeptide includes an amino acid sequence consisting of SEQ ID NO: 1. In some embodiments, the amino acid sequence consisting of SEQ ID NO:1 is encoded by the nucleotide sequence of SEQ ID NO: 20. In embodiments, the variant binds to moesin.

In embodiments, the variants of the present disclosure may include or further include a substitution, deletion, and/or insertion at one or more (e.g. several) positions. In embodiments, the number of amino acid substitutions, deletions, and/or insertions introduced into the mature polypeptide of SEQ ID NOS. 2-14 is up to 3 or up to 5, e.g. 1, 2, 3, 4, 5. In embodiments, the amino acid changes may be of a minor nature, that is conservative amino acid substitutions or insertions that do not significantly affect the folding and/or activity of the protein, small deletions, typically 1-3 amino acids.

In some embodiments, the present disclosure relates to a complementary deoxynucleotide (cDNA) sequence encoding an amino acid sequence having at least at least 90%, at least 95%, or at least 99% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14. In some embodiments, the present disclosure relates to a complementary deoxynucleotide (cDNA) sequence encoding an amino acid sequence consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14. In embodiments, the cDNA are made through laboratory manipulation and genetic engineering techniques. In embodiments, methods of making cDNA's of the present disclosure are known in the art. In embodiments, methods of making synthetic polypeptides of the present disclosure are known in the art.

In some embodiments, the present disclosure relates to a complementary deoxynucleotide (cDNA) sequence encoding an amino acid sequence selected from the group consisting of: SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 14.

In some embodiments, a suitable cDNA sequence of the present disclosure includes the nucleic acid sequence of SEQ ID NO: 18. In embodiments, SEQ ID NO: 18 encodes SEQ ID NO: 2. In some embodiments, SEQ ID NO:19 encodes SEQ ID NO:2.

In some embodiments, the present disclosure includes a polypeptide which is a variant of SEQ ID NO:1, fragment thereof or fusion polypeptide, including: a variant of SEQ ID NO:1 or a fragment thereof having an increased water solubility compared to SEQ ID NO:1 which binds to moesin, wherein the polypeptide includes an affinity tag. In some embodiments, the affinity tag is a polyhistidine tag or a hexahistidine tag. In some embodiments, the affinity tag is bound to an amino or carboxy terminal end of the polypeptide. In some embodiments, the amino acid(s) that form the affinity tag of the present disclosure are inserted at the amino or carboxy terminal end of the polypeptide. In some embodiments, the amino acid(s) that form the affinity tag of the present disclosure are inserted at the carboxy terminal end of the polypeptide. In embodiments, the polypeptides are suitable for use as a moesin binding partner.

In some embodiments, moesin binding partners may be formed by a method for preparing a polypeptide, fragment, or fusion polypeptide or fragment thereof of the present disclosure. In embodiments, a method for preparing a polypeptide, fragment, or fusion polypeptide or fragment thereof includes: a) contacting a polypeptide, fragment, variant, or fusion polypeptide precipitate obtained from bacteria cells with an aqueous urea solution in an amount sufficient to solubilize the precipitate and form a dissolved polypeptide, fragment, variant, or fusion polypeptide within an aqueous medium; b) contacting the dissolved polypeptide, fragment, variant, or fusion polypeptide within the aqueous medium with biotin under conditions sufficient to bind biotin to the dissolved polypeptide, fragment, variant, or fusion polypeptide and form a biotinylated dissolved polypeptide, fragment, variant, or fusion polypeptide; and c) recovering the biotinylated dissolved polypeptide, fragment, variant, or fusion polypeptide as a water-soluble biotinylated polypeptide, fragment, variant, or fusion polypeptide. In some embodiments, the methods include purifying the water-soluble biotinylated polypeptide, fragment, variant, or fusion polypeptide. In some embodiments, purifying includes removing urea or free biotin from the aqueous medium. Optionally, the methods may further include, prior to contacting a polypeptide, fragment, variant, or fusion polypeptide precipitate obtained from bacteria cells with an aqueous urea solution in an amount sufficient to solubilize the precipitate and form a dissolved polypeptide, fragment, variant, or fusion polypeptide within an aqueous medium: providing a nucleic acid sequence encoding a parent polypeptide; modifying the nucleic acid sequence to form a modified nucleic acid sequence encoding a polypeptide, fragment, variant, or fusion polypeptide; introducing the modified nucleic acid sequence into a suitable bacterial host cell; growing the bacterial host cells in a suitable growth medium under conditions leading to expression of the polypeptide, fragment, variant, or fusion polypeptide; and lysing the bacterial host cells to form a polypeptide, fragment, variant, or fusion polypeptide precipitate. In is embodiments, a water-soluble biotinylated polypeptide, fragment, variant, or fusion polypeptide is a moesin binding partner.

In embodiments, the moesin binding partners are formed by a method for preparing a polypeptide, fragment, or fusion polypeptide or fragment thereof. In embodiments, a method may include: (a) providing a nucleic acid sequence encoding a parent polypeptide; (b) modifying the nucleic acid sequence of step (a), to encode a variant, fragment, or fusion polypeptide comprising an affinity tag; (c) introducing the modified nucleic acid sequence of step (b) into a suitable bacterial host cell; (d) growing the bacterial cells in a suitable growth medium under condition leading to expression of the variant, fragment, or fusion polypeptide, wherein the variant, fragment, or fusion peptide comprises an affinity tag; (e) lysing the cells to form a precipitate of the variant, fragment, or fusion polypeptide. In some embodiments, the methods may further include (f) contacting the precipitate with urea in an amount sufficient to solubilize the variant, fragment, or fusion polypeptide and then with a labelling moiety such as biotin; (g) eluting the variant, fragment, or fusion polypeptide; and (h) purifying the variant, fragment, or fusion polypeptide, and optionally forming a substantially purified variant, fragment, or fusion polypeptide.

In some embodiments, the present disclosure relates to a nucleic acid encoding a moesin binding partner, a vector including a nucleic acid encoding a moesin binding partner, or a bacterial host cell including a nucleic acid encoding a moesin binding partner.

In embodiments, nucleic acids and expression vectors can be generated via methods known per se for modifying nucleic acids. Such methods are, for example, presented in relevant manuals such as the one by Fritsch, Sambrook and Maniatis, “Molecular cloning: a laboratory manual”, Cold Spring Harbor Laboratory Press, New York, 1989, and familiar to a person skilled in the art in the field of biotechnology. Examples of such methods are chemical synthesis or the polymerase chain reaction (PCR), optionally in conjunction with further standard methods in molecular biology and/or chemistry or biochemistry.

In embodiments, a bacterial host cell containing an expression vector according to the present disclosure is provided. In embodiments, an expression vector is introduced into the host cell by the transformation thereof. For example, transforming a vector according to the present disclosure into a microorganism, which then constitutes a host cell according to the present disclosure. Alternatively, it is also possible for individual components, i.e., nucleic acid portions or fragments, for example the components of a vector according to the present disclosure to be introduced into a host cell in such a way that the thus resulting host cell includes a vector according to the present disclosure.

Methods for transforming cells are established in the prior art and well known to a person skilled in the art. Prokaryotic cells, are suitable as host cells as they can be advantageously manipulated genetically, for example with regard to transformation with the vector and the stable establishment thereof. In addition, host cells may be easily manipulatable from a microbiological and biotechnological perspective, for example, ease of culture, high growth rates, low demands on fermentation media, and good production for foreign proteins.

In embodiments, host cells are prokaryotic or bacterial cells. Bacteria have short generation times and low demands in terms of culture conditions. As a result, it is possible to establish cost-effective methods. In addition, a wealth of experience is available to a person skilled in the art in the case of bacteria in fermentation technology. For a specific production process, Gram-negative or Gram-positive bacteria may be suitable for a very wide variety of different reasons which are to be determined experimentally on an individual basis, such as nutrient sources, rate of product formation, time requirement, etc.

In embodiments, the host cell is a bacterium, such as one selected from the group of the genera of Escherichia, Klebsiella, Bacillus, Staphylococcus, Corynebacterium, Arthrobacter, Streptomyces, Stenotrophomonas and Pseudomonas, or one selected from the group of Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxidans, Streptomyces lividans, Streptomyces coelicolor and/or Stenotrophomonas maltophilia.

In embodiments, the host cells can be modified with respect to their requirements in terms of culture conditions, can have other or additional selection markers, antibiotic selection marker(s), or can express other or additional proteins. More particularly, the host cells can be those which express multiple proteins or polypeptides.

In embodiments, the host cells are cultured and fermented in a manner known in the art, for example in batch systems or continuous systems. In some embodiments, host cells are used to prepare proteins encoded by the nucleic acid sequence encoding one or more moesin binding partners of the present disclosure. The disclosure therefore further provides a method for producing a water-soluble polypeptide in a bacterial host cell, the method including: transforming one or more bacterial host cells with an expression vector comprising a nucleotide sequence encoding a moesin binding partner; culturing the transformed bacterial host cells in a medium suitable for expression of the moesin binding partner, and dissolving the moesin binding partner in an aqueous urea mixture including a detectable label moiety to form a water-soluble labelled moesin binding partner. In some embodiments, the detectable label moiety is biotin.

In some embodiments, the present disclosure relates to a synthetic biotinylated variant probe, including an amino acid sequence including a moesin binding partner, wherein the moesin binding partner includes a detectable label moiety.

In some embodiments, a moesin binding partner includes one or more antibodies that bind to moesin or a segment thereof. In embodiments, one or more antibodies may be used for the detection of intracellular and/or cell surface moesin on lymphocytes by known methods such as flow cytometry. For example, in embodiments, lymphocytes may be reacted with polyclonal rabbit anti-moesin antibody (amino acids 1 to 294, Proteintech, #16495-1-AP) for a duration such as 30 minutes at room temperature in the dark. The cells may be washed and incubated with e.g., (1:100) goat anti-rabbit IgG highly cross-adsorbed secondary antibody conjugated with Alexa Fluor 488 (Thermo Fisher Scientific, #A-11034) for a duration such as 30 minutes at room temperature in the dark. In embodiments, the lymphocytes may be washed again and stained with anti-CD16 PE, anti-OD3 PE, or anti-CD19 PE mouse monoclonal antibodies (BD Pharmingen™, #555413) for 30 minutes at room temperature in the dark. Finally, lymphocytes may be washed, resuspended with, and analyzed on a CytoFLEX flow cytometer, Beckman Coulter Life Sciences, Indianapolis, IN. In embodiments, antibodies may contact nNK cells under conditions suitable for binding to moesin or a region thereof. In embodiments, one or more antibodies bind to moesin or a region thereof under conditions suitable to signal or indicate the binding of the antibody to moesin of an nNK cell. In embodiments, antibodies suitable for use herein may be preselected to bind to moesin. In embodiments, antibody suitable for use herein may be characterized as polyclonal or is monoclonal.

In some embodiments, the present disclosure relates to a method for determining the number of naïve natural killer (NK) cells in a biological sample, including: (a) contacting a sample containing naïve NK cells with one or more moesin binding partners including a detectable label moiety and having specific binding affinity for moesin, under conditions permissive for binding of the one or more moesin binding partners expressed by naïve NK cells in the sample; and (b) counting a number of cells bound by the one or more moesin binding partners to determine the number of naïve natural killer cells in the sample. In some embodiments, the one or more moesin binding partners are characterized as variant polypeptides. In some embodiments, the detectable label moiety is biotin.

In some embodiments, the present disclosure relates to a method for identifying or determining a number of naïve natural killer (NK) cells in a biological sample, including: (a) contacting a sample containing naïve NK cells with one or more moesin binding partners including a detectable label moiety and having specific binding affinity for moesin, under conditions permissive for binding of the one or more moesin binding partners expressed by naïve NK cells in the sample, wherein the one or more moesin binding partners are an antibody with a binding affinity to moesin, or a synthetic biotinylated variant probe configured to bind moesin; and (b) detecting the moesin binding partners to identify or determine a number of naïve natural killer cells in the sample.

In some embodiments, the present disclosure relates to a method of identifying cytotoxic naïve natural killer (NK) cells, including: (a) contacting a sample containing naïve NK cells with one or more moesin binding partners including a detectable label moiety and having specific binding affinity for moesin, under conditions permissive for binding of the one or more moesin binding partners expressed by naïve NK cells in the sample, wherein the one or more moesin binding partners are an antibody with a binding affinity to moesin, or a synthetic biotinylated variant probe configured to bind moesin; and (b) detecting the moesin binding partners to identify or determine a presence of cytotoxic naïve natural killer cells.

In some embodiments, a recombinant polypeptide or protein is produced in a bacterial expression system including an affinity tag on the C-terminus of a fusion polypeptide or fusion protein. For example, a vector with a 6× histidine peptide could be used for affinity purification of the soluble recombinant polypeptide or protein. Specifically, a cDNA encoding amino acids 79 to 197 of the Haymaker ORF may be cloned into the pET28a+ vector. In some embodiments, a positive clone may be selected, and the cDNA transferred into a BL21-CodonPlus (DE3)-RIPL, expression vector. In some embodiments, the recombinant polypeptide or protein is produced by induction with β-d-1-thiogalactopyranoside (IPTG). In some embodiments, the induced bacteria are lysed and a precipitate of the recombinant protein forms. The precipitate is washed to remove contaminating soluble proteins 3 times in binding buffer. In embodiments, a urea solution, such as 8M urea, is added to the precipitate in order to solubilize the precipitate. In embodiments, the recombinant polypeptide or protein is then purified by affinity on a nickel resin (NEBExpress™ Ni Resin) by the standard batch method. In some embodiments, the recombinant polypeptide or protein is eluted with a 500 mM imidazole-8M urea borate buffer and the preparation is dialyzed in a urea borate buffer to remove imidazole. In some embodiments, the preparation is subsequently treated with 3.0 mM Biotin-NHS, water soluble (Sigma-Aldrich 203118) for one hour followed by dialysis with a NaCl-borate buffer to remove urea and free Biotin-NHS. Unexpectedly, the biotinylated recombinant protein remains soluble after removal of urea. It is noteworthy that the recombinant polypeptide or protein precipitates if the preparation is not first treated with Biotin-NHS. The soluble biotinylated recombinant polypeptide or protein of the present disclosure may be subsequently used in this form for down-stream assays that are performed in aqueous solutions.

In some embodiments, the present disclosure includes a method of isolating one or more naïve NK cells or NK cells from a blood sample and/or separating one or more naïve NK cells or NK cells from T and B cells. For example, in some embodiments, naïve NK cells or NK cells are isolated or substantially purified by contacting one or more moesin binding partners with one or more substrates such as magnetic beads under conditions sufficient to form one or more conjugated substrates or conjugated magnetic beads. In some embodiments, magnetic particles may be conjugated to antibodies recognizing moesin. In some embodiments, magnetic particles may be conjugated to one or more moesin binding partners. In embodiments, the one or more moesin binding partners are conjugated or attached to one or more magnetic beads using methods known in the art. Non-limiting examples of reagents, magnetic particles, cell separation methods, and conditions where polypeptides and is proteins are conjugated, fixed, or attached to a substrate are described in U.S. Pat. Nos. 5,543,289, 9,885,032, 10,006,840, and 10,620,212 (all of which are herein incorporated by reference in their entireties). Subsequently, the process sequence may include contacting the one or more conjugated magnetic beads with a blood sample under conditions sufficient to form one or more magnetic bead/naïve NK cell complexes. In embodiments, the process sequence further includes separating the one or more magnetic bead/naïve NK cell complexes to isolate the naïve NK cells from the blood sample.

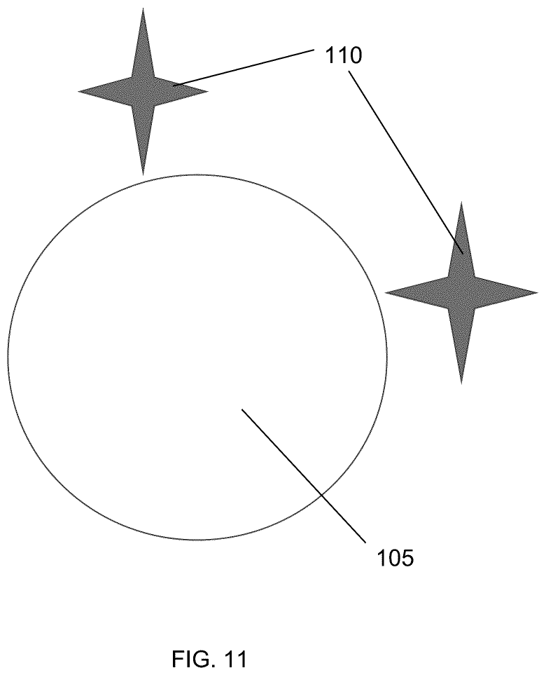

In some embodiments, the present disclosure relates to a bead including: a substrate; and one or more moesin binding partners. For example, referring to FIG. 11, a bead composition 100 is shown including substrate 105, and one or more moesin binding partners 110. In some embodiments, the one or more moesin binding partners 110 include one or more polypeptides with moesin binding affinity fixedly attached to the substrate 105 or one or more anti-moesin antibodies fixedly attached to the substrate 105. In some embodiments, the one or more polypeptides with moesin binding affinity fixedly attached to the substrate is one or more HYMKR polypeptides or one or more fragments thereof. In some embodiments, the one or more polypeptides with moesin binding affinity fixedly attached to the substrate incudes a polypeptide which is a variant of SEQ ID NO:1, a fragment thereof, or fusion polypeptide, including: a variant of SEQ ID NO:1 or a fragment thereof having an increased water solubility compared to SEQ ID NO:1 which binds to moesin. In some embodiments, the one or more polypeptides with moesin binding affinity fixedly attached to the substrate comprises a variant of a parent HYMKR polypeptide including: a plurality of deletions of amino acid residues corresponding to amino acid residues M1 to P78 and D198 to G361 using SEQ ID NO:1 for numbering. In some embodiments, the substrate is a magnetic particle. In some embodiments, the substrate is a spherical magnetic particle.

Still referring to FIG. 11, in some embodiments, the substrate 105 includes, a one or more sepharose beads, one or more magnetic beads, or combinations thereof. In some embodiments, proteins or amino acid sequences of the present disclosure may be attached to the one or more sepharose beads by methods known in the art including the methods described in Das et al., A Novel Ligand in Lymphocyte-mediated Cytotoxicity: Expression of the Beta Subunit of H+ Transporting ATP Synthase on the Surface of Tumor Cell Lines, J. Exp. Med. 180(1): 273-281 (1994), see for example, the second full paragraph on page 274 describing coupling protein to CNBR-Sepharose 4B at 4° C. for 90 min in PBS containing 1% BSA. Here, the reacted beads were washed three times with buffer and incubated for 90 min with biotinylated tumor plasma membrane proteins. The beads were then washed three times with PBS containing 1% BSA and two times with PBS alone, suspended in Laemmli SDS-PAGE sample buffer, and boiled for 10 min. The supernatant was subjected to SDS-PAGE and proteins from gels were transferred to Immobilon-P membrane by electroblotting. The immunoprecipitated biotinylated tumor membrane proteins were identified on blots by reaction with a streptavidin-biotin detection system. In embodiments, this methodology is suitable for use in attaching proteins, or amino acids of the present disclosure to a sepharose substrate or sepharose bead.

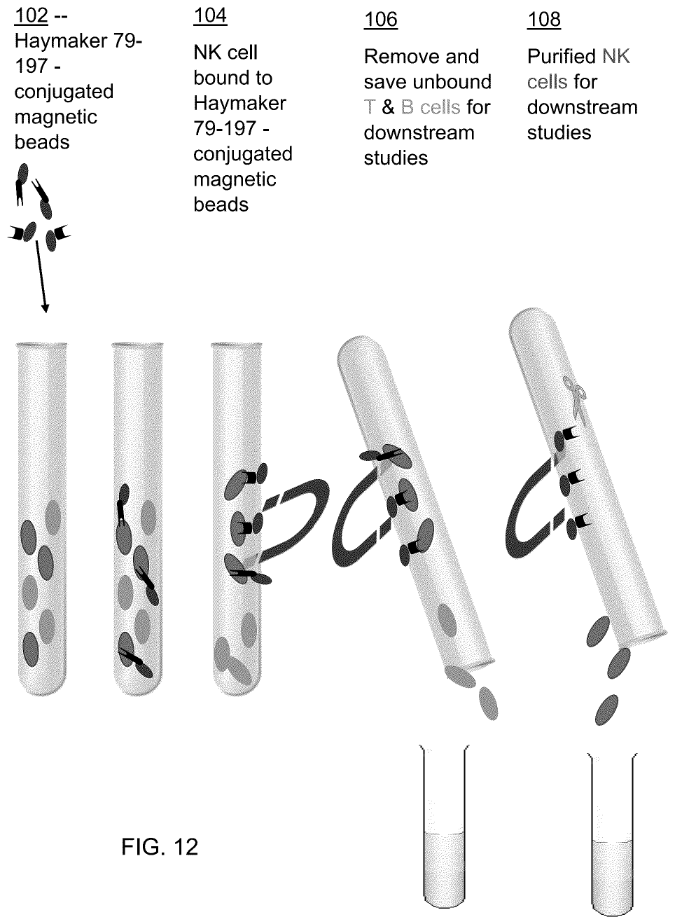

Referring now to FIG. 12, a purification process flow of the present disclosure is shown. In embodiments, naïve natural killer cells (nNK cells) and/or NK cells may be purified, substantially purified, and/or separated from T and B cells by a selection process in accordance with the present disclosure. Referring to FIG. 12 at process sequence 102 a binding partner in accordance with the present disclosure such as e.g., Haymaker 79-197 is bound to a substrate, or solid support (or matrix) such as a magnetic bead to form a magnetically responsive material or composition configured for separation of cells for positive or negative selection. In embodiments, the sample or composition of cells to be separated is incubated with the magnetically responsive material under conditions suitable for suitable for binding or attaching the magnetically responsive material which may be configured to specifically bind to moesin present on one or more cells or cell surfaces, or a population of cells that it is desired to separate, e.g., it is desired to negatively or positively select. In embodiments, the incubation may be performed under conditions wherein naïve natural killer cells (nNK cells) including moesin will specifically bind to the magnetically responsive material including the binding partner of the present disclosure. Accordingly, moesin will attach to the magnetically responsive material and the cell surface including moesin will be affixed to the magnetically responsive material.

At process sequence 104, the sample is placed in a magnetic field, and those cells having magnetically responsive or magnetizable particles attached thereto will be attracted to the magnet and separated from any cells in the sample that do not include moesin, such as T and B cells. In embodiments, process sequence 106 shows an example of a positive selection wherein cells attracted to the magnet remain in a sample tube or container, while cells not attracted to the magnet (such as T and B cells devoid of moesin) may be eluted away. Cells eluted away may be characterized as cells purified by a negative selection process.

Referring now to process sequence 108, magnetically responsive particles are removed from the cells, and the cells may be collected, isolated, substantially purified, and/or further used e.g., in downstream studies. Removing the magnetizable particles from the cells may be performed by methods known in the art, such as biodegrading the magnetizable particles. In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. See e.g., U.S. Pat. No. 10,786,533 (herein incorporated by reference in its entirety).

In some embodiments, the present disclosure includes a method of selecting or substantially purifying T and B cells, from a mixture of cells, including: contacting a mixture of cells with the bead of the present disclosure under conditions that bind the bead to one or more nNK cells within the mixture of cells, if any, within a container; immobilizing the bead of within the container; and eluting T and B cells from the mixture of cells, if any. In some embodiments, the bead is characterized as a magnetic bead, and immobilizing further includes contacting the magnetic bead with a magnetic field under conditions sufficient to hold the magnetic bead. In some embodiments, the substrate of the bead is sepharose.

### EXAMPLES

Summary: Moesin: a novel receptor on Natural Killer lymphocytes binds to TOMM40 on leukemia cells initiating cytolysis. Moesin-Haymaker now provides a newly discovered pathway for delivery of a lethal hit to leukemia by unstimulated Natural Killer cells.

### Materials and Methods

### HYMKR⁷⁹⁻¹⁹⁷ Construction in a Bacterial Expression Vector System

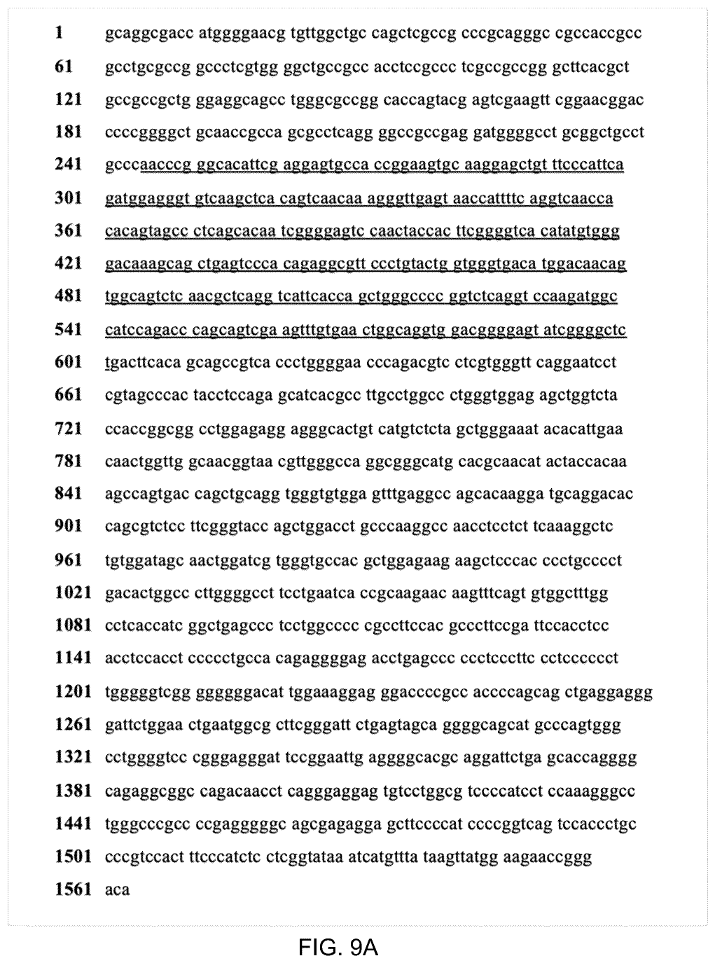

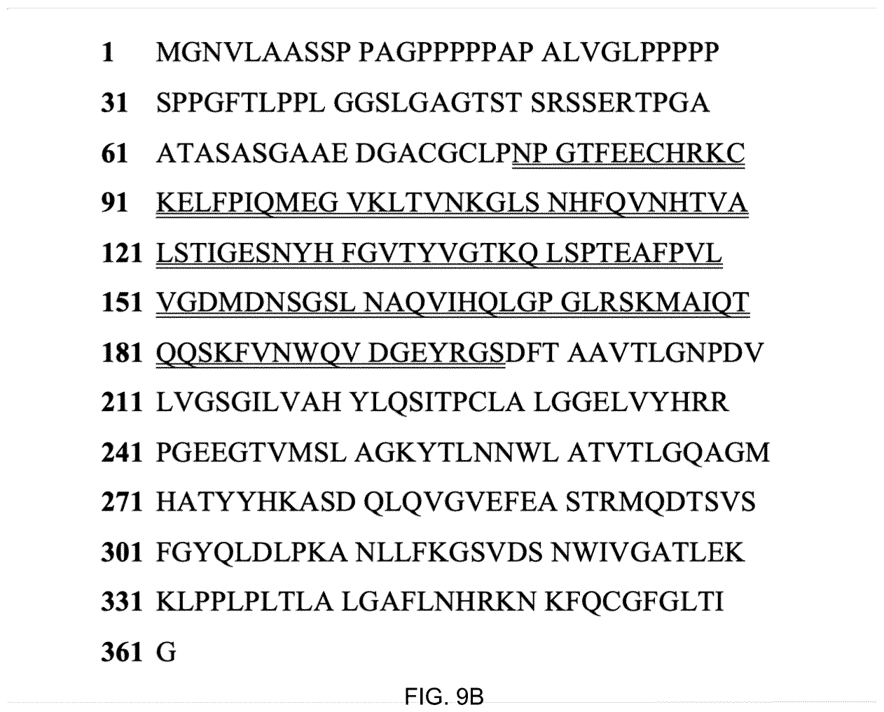

Nucleic acids encoding amino acid N79 to S197 of Haymaker/TOMM40 (HYMKR) protein (SEQ ID NO: 2) was cloned into the pET28a⁺ vector (Novagen, #69864). The 5369 base pair vector encodes a C-terminal His-tag on the recombinant protein and a kanamycin (Kan) resistance gene. PCR was performed to amplify the selected region by using the cDNA of the ORF of HYMKR as the template (GenBank accession no. AF316402) (See e.g., FIG. 9A). More specifically, FIGS. 9A and 9B depict, respectively, a Nucleotide and amino acid sequences of HYMKR/Tomm40. Referring to FIG. 9A, the underlined nucleotide sequence (GenBank accession no. AF316402) encodes amino acids N79 to S197 of HYMKR (SEQ ID NO: 2). The amino acid sequence of HYMKR (GenBank accession no. AAL46627) is also shown. The double-underlined amino acid sequence indicates the composition of the HYMKR polypeptide from N79 to S197 that was used in embodiments, of the present disclosure. The nucleotides that encode this region were cloned into the pET28a⁺ vector, which resulted in a 6×His-tag fused to the carboxyl terminus of the recombinant HYMKR polypeptide (HYMKR⁷⁹⁻¹⁹⁷-6×His) see e.g., SEQ ID NO: 14.

Forward and reverse primers were designed and then obtained from Invitrogen (Carlsbad, CA). These primers contained a replacement for two arginine codons, CGG with CGT, since tRNAs for the former codon are rare in E. coli. The nucleotide sequences of the primers are indicated:

forward:
(SEQ ID NO: 16)
5′-CCATAACCATGGGTAACCCGGGCACATTCGAGGAGTGCCACCGT-
3′;
reverse:
(SEQ ID NO: 17)
5′ CCATATCTCGAGAGAGCCACGATACTCCCCGTCCAC-3′.

HYMKR cDNA (see e.g., FIG. 9A) encoding amino acids 79 to 197 (HYMKR⁷⁹⁻¹⁹⁷ or SEQ ID NO:2) were inserted between the NcoI and XhoI restriction sites (New England BioLabs, #R0193S and #R0146S). Following ligation, the recombinant plasmid DNA was transformed into Nova Blue E. coli competent cells (Novagen, #70181) using heat shock. The transformation mixture was plated on selective LB agar containing 50 μg/mL of Kan and incubated at 37° C. overnight. Selected colonies were inoculated separately in 3 mL LB-Kan culture medium and incubated at 37° C. shaking overnight. Plasmid DNA was purified using the QIAquick Spin Miniprep Kit (Qiagen, #27104) and submitted for DNA sequencing to ACGT, Inc. (Wheeling, IL). The confirmed, purified recombinant plasmid DNA from the Nova Blue E. coli were transformed into BL21-CodonPlus (DE3)-RIPL E. coli competent cells (Agilent Technologies, #230240) using heat shock.

### Induction of HYMKR⁷⁹⁻¹⁹⁷ Polypeptide in Transformed (DE3)-RIPL E. Coli

The HYMKR⁷⁹⁻¹⁹⁷ polypeptide was induced in the transformed cells that contained the recombinant plasmid. Transformed (DE3)-RIPL E. coli cells were grown overnight in LB broth with Kan and chloramphenicol (LB-Kan-CHL) LB medium. 50 ml of LB-Kan culture medium was inoculated with 3 mL of overnight culture and grown for 2 hours at 37° C. Cells were then incubated with 1.0 mM Isopropyl β-D-1-thiogalactopyranoside (IPTG, Millipore Sigma, #367-93-1) to induce expression of the recombinant polypeptide. The culture was incubated at 37° C. with shaking for 2 hours, then at 4° C. overnight prior to harvest.

Extraction of HYMKR⁷⁹⁻¹⁹⁷ polypeptide from induced (DE3)-RIPL E. coli

HYMKR⁷⁹⁻¹⁹⁷ was extracted from the induced cells with the bacterial protein extraction reagent (B-PER, Thermo Fisher Scientific, #78248), 10 mg/mL lysozyme (Millipore Sigma, #L3790), and 1 μg/mL DNase I (New England BioLabs, #M0303S). Additionally, cells were freeze-thawed three times to improve extraction efficiency. Then the bacterial lysate was centrifuged at 2000 g for 10 minutes. The supernatant was discarded and the HYMKR⁷⁹⁻¹⁹⁷ precipitate was washed with borate buffer (10 mM sodium borate, 10 mM benzamidine, 1 mM EDTA, 1 mM iodoacetamide, 1 mM phenylmethylsulfonyl fluoride, pH 9.0). The expressed HYMKR⁷⁹⁻¹⁹⁷ polypeptide was then dissolved in borate buffer by addition of 8 M urea.

### Purification of HYMKR⁷⁹⁻¹⁹⁷ Polypeptide on a Ni-NTA Agarose Resin

HYMKR⁷⁹⁻¹⁹⁷ was purified on a Ni-NTA agarose resin (Ni²⁺ resin), batch method (Qiagen, ID:30210) via the C-terminal His-tag. The resin was prepared by washing twice with His-tag equilibration buffer containing 8 M urea. Solubilized HYMKR⁷⁹⁻¹⁹⁷ was then added to the washed resin and incubated for 2 hours at room temperature with gentle shaking. The reaction mixture was centrifuged at 500 g for 5 minutes, and the supernatant was collected and stored for further analysis. The resin was then washed four times with His-tag washing buffer containing 8 M urea. His-tag imidazole elution buffer containing 8 M urea was added to the resin and incubated for 1 hour at room temperature with gentle shaking. The reaction mixture was then centrifuged at 500 g for 5 minutes, and the supernatant that contained the eluted HYMKR⁷⁹⁻¹⁹⁷ was collected. The eluate was dialyzed against borate buffer containing 8 M urea for 2 hours to remove imidazole.

### Biotinylation of HYMKR⁷⁹⁻¹⁹⁷ Polypeptide

Affinity purified HYMKR⁷⁹⁻¹⁹⁷ was biotinylated by incubation with 3 mM biotin 3-sulfo-N-hydroxysuccinimide ester sodium salt (Sigma-Aldrich, #B5161) for 1 hour at room temperature with gentle shaking. (See e.g., Das, B., et al., Preferential interaction of a novel tumor surface protein (p38.5) with naïve natural killer cells. J Exp Med, 1997. 185(10): p. 1735-42; von Boxberg, Y., R. Wutz, and U. Schwarz, Use of the biotin-avidin system for labelling, isolation and characterization of neural cell-surface proteins. Eur J Biochem, 1990. 190(2): p. 249-56; and Meier, T., et al., Immunodetection of biotinylated lymphocyte-surface proteins by enhanced chemiluminescence: a nonradioactive method for cell-surface protein analysis. Anal Biochem, 1992. 204(1): p. 220-6.) Biotinylated-HYMKR⁷⁹⁻¹⁹⁷ was then dialyzed three times against 200 ml of borate buffer to remove biotin and urea. Sufficient NaCl was added to bring the solution to 0.9% saline. The soluble biotinylated-HYMKR⁷⁹⁻¹⁹⁷ was stored at −20° C. for use in downstream experiments.

### Lymphocyte Purification

Human peripheral blood lymphocytes (HPBL) were obtained by venipuncture from volunteers and isolated using Ficoll-Paque™ PLUS (GE Healthcare Life Sciences, #17144002). This procedure was reviewed and approved by SUNY Downstate Health Sciences University Institutional Review Board & Privacy Board (Downstate IRB #1668790-2). Blood was diluted 1:1 with Hank's balanced salt solution (HBSS) without Ca²⁺ and Mg²⁺ (Stemcell Technologies, #37250). The diluted blood was then layered on Ficoll-Paque and centrifuged at 400 g for 30 minutes. The top layer of plasma and platelets was discarded. The resuspended mononuclear cell layer was transferred into a clean tube, washed twice with HBSS, resuspended in Dulbecco's Phosphate Buffered Saline (DPBS) with 2.5% Fetal Bovine Serum (FBS) as a source of IgG and 0.025% Sodium Azide, SA (DPBS-2.5% FBS-0.25% SA) for use in flow cytometry experiments. Furthermore, approximately half of the isolated mononuclear cells were permeabilized with the FIX & PERM™ Cell Permeabilization Kit (Thermo Fisher Scientific, #GAS003) and used in flow cytometry studies to detect the intracellular target protein. The remaining fraction of non-permeabilized cells were used in flow cytometry studies to detect cell surface target protein.

### Isolation of nNK Cells

nNK cells were isolated using negative selection with the EasySep™ Direct Human NK Cell isolation kit (Stemcell Technologies, #19665). Isolated mononuclear cells from the Ficoll-Paque preparation were incubated with the isolation Cocktail, the RapidSphere™ reagent, and DPBS with 2% FBS (Stemcell Technologies, #07905) for 5 minutes at room temperature. The tube containing the cells and magmatic beads was placed into a magnetic rack and incubated for another 5 minutes at room temperature. The enriched nNK cell suspension was pipetted into a fresh tube for a second incubation in the magnetic rack. The isolated nNK cells were collected, washed, and resuspended in DPBS without Ca²⁺ and Mg²⁺ (Thermo Fisher Scientific, #14190136). The Ficoll-Paque™ fractionated mononuclear cells contained about 2.5% nNK. After the mononuclear cells were isolated by negative selection, nNK cells were about 90% of the total cell suspension according flow cytometry (data not shown).

### Biotinylation of nNK Cell Surface Proteins

Surface proteins of freshly isolated nNK cells were biotinylated by a previously published method (See e.g., von Boxberg, Y., R. Wutz, and U. Schwarz, Use of the biotin-avidin system for labelling, isolation and characterization of neural cell-surface proteins. Eur J Biochem, 1990. 190(2): p. 249-56; Meier, T., et al., Immunodetection of biotinylated lymphocyte-surface proteins by enhanced chemiluminescence: a nonradioactive method for cell-surface protein analysis. Anal Biochem, 1992. 204(1): p. 220-6; and Das, B., et al., A novel ligand in lymphocyte-mediated cytotoxicity: expression of the beta subunit of H+ transporting ATP synthase on the surface of tumor cell lines. J Exp Med, 1994. 180(1): p. 273-81.) In brief cells were incubated with the lipid insoluble reagent, 3 mM Biotin 3-sulfo-N-hydroxysuccinimide ester sodium salt for 1 hour at room temperature with gentle shaking (See e.g., Das, B., et al., Preferential interaction of a novel tumor surface protein (p38.5) with naive natural killer cells. J Exp Med, 1997. 185(10): p. 1735-42). Surface-biotinylated nNK cells were washed twice with 1 M Glycine in DPBS, pH 7.4 and three times with 10 mM Hepes, 145 mM NaCl, 4 mM KCl, 11 mM Glucose, pH 7.4 to remove the unreacted biotin, and then resuspended in 1% Triton-X 100 borate buffer containing 1 μg/mL DNase I (New England BioLabs, #M0303S). The cell lysate was then dialyzed twice against borate buffer each for 2 hours and a third time against borate buffer containing 0.05% Triton-X 100 for another 2 hours.

### Binding of Biotinylated-HYMKR⁷⁹⁻¹⁹⁷ to Lymphocyte Subpopulations

Non-permeabilized unstimulated lymphocytes (100 μl of 1×10⁶ cell/mL) were reacted with 75 μg of biotinylated-HYMKR⁷⁹⁻¹⁹⁷ for 30 minutes at room temperature in the dark. The cells were washed three times with DPBS-2.5% FBS-0.025% SA and probed with (1:200) streptavidin-FITC (BD Pharmingen™, #554060) for 30 minutes at room temperature in the dark. They were then washed three times with DPBS-2.5% FBS-0.025% SA and stained with (1:10) anti-CD16 PE or anti-CD3 PE mouse monoclonal antibodies (BD Pharmingen™, #555407 and #555333, respectively). Finally, the reacted cells were washed three times, resuspended in DPBS-2.5% FBS-0.025% SA, and analyzed on a CytoFLEX flow cytometer (Beckman Coulter Life Sciences, Indianapolis, IN). Experiments from three subjects are shown.

Identification of nNK Cell-Binding Protein to the HYMKR⁷⁹⁻¹⁹⁷ Ni²⁺ Resin

Dialyzed surface-biotinylated or non-biotinylated nNK cell lysate was applied to the HYMKR⁷⁹⁻¹⁹⁷ bound to the Ni²⁺ resin (HYMKR⁷⁹⁻¹⁹⁷-Ni²⁺ resin) and incubated overnight at 4° C. with gentle shaking in the dark. The following day, the reaction mixture was centrifuged at 500 g for 5 minutes and washed twice with borate buffer. His-tag imidazole elution buffer containing 8 M urea was added and incubated for 1 hour at room temperature with gentle shaking in the dark. The reaction mixture was then centrifuged at 500 g for 5 minutes, and the supernatant that contained the eluted HYMKR⁷⁹⁻¹⁹⁷ and any nNK protein which had bound to the affinity resin was collected. The eluate was concentrated using the Amicon ultra-0.5 centrifugal filter unit (Millipore Sigma, #UFC501024) and solubilized in Laemmli sample buffer with 2% mercaptoethanol (See e.g. Das, B., et al., A novel ligand in lymphocyte-mediated cytotoxicity: expression of the beta subunit of H+ transporting ATP synthase on the surface of tumor cell lines. J Exp Med, 1994. 180(1): p. 273-81). SDS-PAGE was then performed, and a Western blot of the surface-biotinylated nNK cell proteins was probed with streptavidin-alkaline phosphatase and developed with the 1-Step™ NBT/BCIP substrate solution to reveal the molecular weight of nNK protein(s) that had bound to the HYMKR⁷⁹⁻¹⁹⁷-Ni²⁺ resin.

Amino Acid Sequence of Peptides from the Putative nNK Receptor for HYMKR

Two preparation were sequenced, one contained only non-biotinylated nNK cell surface proteins. While the second preparation that was sequenced contained biotinylated nNK cell surface proteins in addition to the non-biotinylated nNK cell proteins. The concentrated nNK plasma membrane protein preparation was separated on SDS-PAGE. Thin slices of the gel between 60 kDa and 72 kDa were removed and sent for peptide sequencing (Harvard University Center for Mass Spectrometry Proteomics Portal, Cambridge, MA). Sequencing was performed by liquid chromatography with tandem mass spectrometry (LC-MS/MS).

### Detection of Cell Surface and Intracellular Moesin on Lymphocyte Subpopulations by Flow Cytometry

Permeabilized or non-permeabilized unstimulated lymphocytes (100 μl of 1×10⁶ cells/mL) were reacted with 0.3 μg of anti-moesin rabbit polyclonal antibody (the immunogen, amino acids 1 to 294, Proteintech, #16495-1-AP) for 30 minutes at room temperature in the dark in DPBS-2.5% FBS-0.25% SA. Lymphocytes were washed three times with DPBS-2.5% FBS-0.025% SA. The cells were then incubated with (1:100) goat anti-rabbit IgG-Alexa Fluor 488 (Thermo Fisher Scientific, #A-11034) for 30 minutes at room temperature in the dark. The lymphocytes were washed three times with DPBS-2.5% FBS-0.025% SA, resuspended in the latter buffer and stained with anti-CD16 PE, anti-CD3 PE, or anti-CD19 PE mouse monoclonal antibodies (BD Pharmingen™, #555407, #555333, or #555413, respectively) for 30 minutes at room temperature in the dark. Finally, lymphocytes were washed three times, resuspended with DPBS-2.5% FBS-0.025% SA, and analyzed on a CytoFLEX flow cytometer.

Immunologic Identification of the 70 kDa Protein that was Purified by Affinity Chromatography on the HYMKR⁷⁹⁻¹⁹⁷-Ni²⁺ Resin

A pulldown of non-biotinylated nNK cell lysate using the HYMKR⁷⁹⁻¹⁹⁷-Ni²⁺ resin was performed as described above. The eluate containing the captured nNK plasma membrane protein was run on SDS-PAGE (15% running gel-4% stacking) and transferred to a 0.45 μm polyvinylidene fluoride (PVDF) membrane Immobilon®-P (Millipore Sigma, #IPVH00010). Anti-moesin rabbit polyclonal antibody at 1:1000 dilution of a 0.15 mg/ml solution was incubated overnight at 4° C. with gentle shaking in the dark. The membrane was incubated with secondary (1:1000) goat anti-rabbit IgG-alkaline phosphatase (Millipore Sigma, #A3687) for 1 hour at room temperature with gentle shaking in the dark. Detection was performed with 1-Step™ NBT/BCIP substrate solution (Thermo Fisher Scientific, #34042) incubated for 2 minutes at room temperature with gentle shaking in the dark.

### Statistical Analysis

Statistical analyses were performed using GraphPad Prism 8 (GraphPad Software, La Jolla, CA). Sample size calculations were not performed. The mean and standard deviation was determined (n=3, df=4). The null hypothesis: NK cells are more reactive than T cells or B cells. A one-tailed student's t-test for unpaired two sample means was used to establish statistical significance where a p value of less than 0.05 was considered statistically significance.

### Results

In order to develop tools for identifying the nNK receptor for HYMKR, a cDNA of the Open Reading Frame (ORF) of this protein (See e.g., Das, B., et al., Genetic identity and differential expression of p38.5 (Haymaker) in human malignant and nonmalignant cells. Int J Cancer, 2001. 94(6): p. 800-6) was used as a template to clone the nucleotide sequence encoding amino acids 79 to 197 into the pET28a⁺ expression vector as described in Materials and Methods. This maneuver resulted in a 6×His-tag fused to the carboxyl terminus of the recombinant polypeptide. This region of HYMKR was selected based on blocking studies that suggested it contained the binding site for the nNK cell receptor (See e.g., Das, B., R. Mushnitsky, and A. J. Norin, Difference in target cell recognition of naive and activated human natural killer cells: role of Haymaker (p38.5) in tumoricidal activity. Hum Immunol, 2005. 66(3): p. 241-51). The 79-197 polypeptide was induced in BL21-CodonPlus (DE3)-RIPL E. coli and purified on a Ni-NTA agarose resin (Ni²⁺ resin), which is referred to as “HYMKR⁷⁹⁻¹⁹⁷” (SEQ ID NO: 2). This construct was used in downstream experiments.

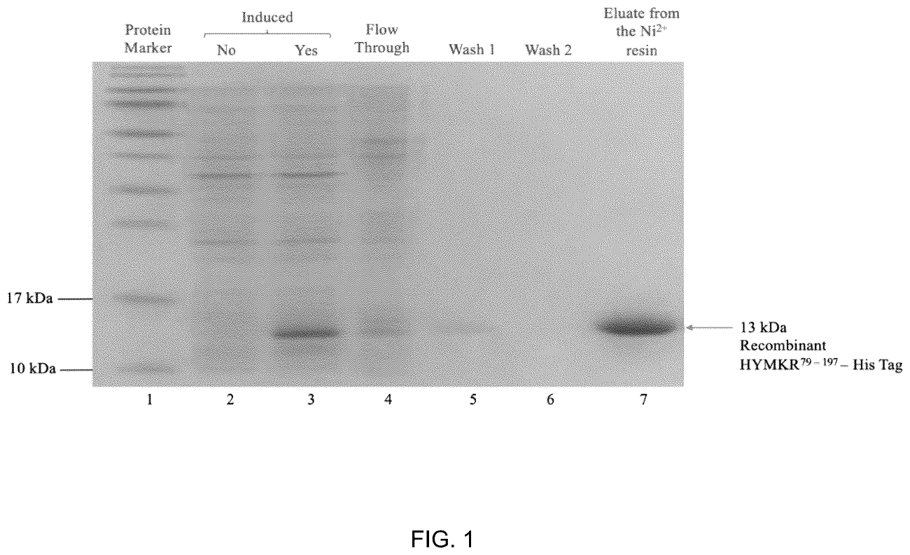

More specifically, referring now to FIG. 1, Coomassie blue stained SDS-PAGE of uninduced and induced recombinant HYMKR polypeptide (HYMKR⁷⁹⁻¹⁹⁷) is shown. The induced cell lysate shows a prominent band of about 13 kDa (lane 3) compared to the uninduced cell lysate (lane 2). The affinity-purified recombinant polypeptide was eluted from the Ni-NTA agarose resin (Ni²⁺ resin) with imidazole buffer as shown in lane 7. Note that only a single band was detected at the predicted molecular weight of HYMKR⁷⁹⁻¹⁹⁷ without any other observable proteins on the gel.

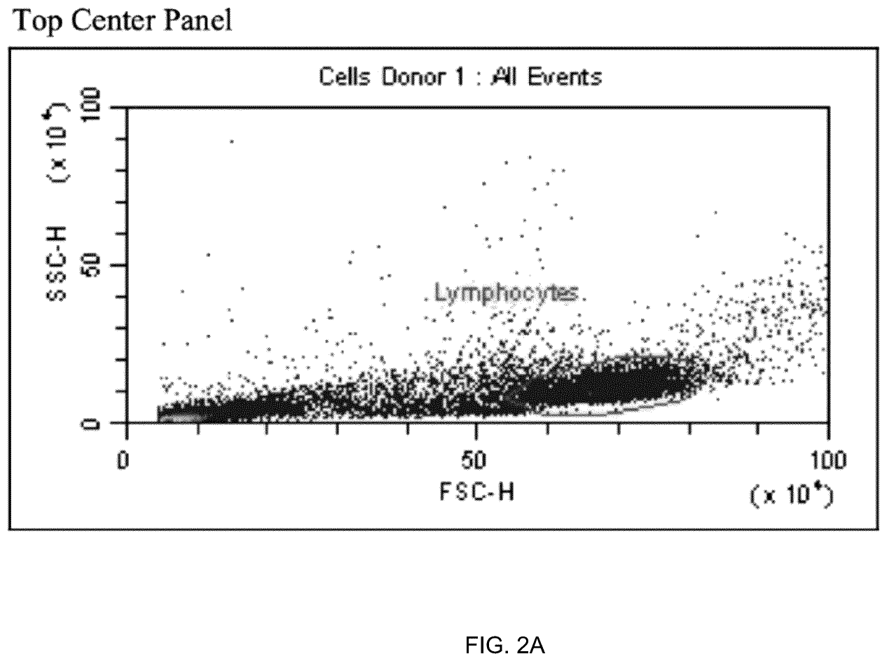

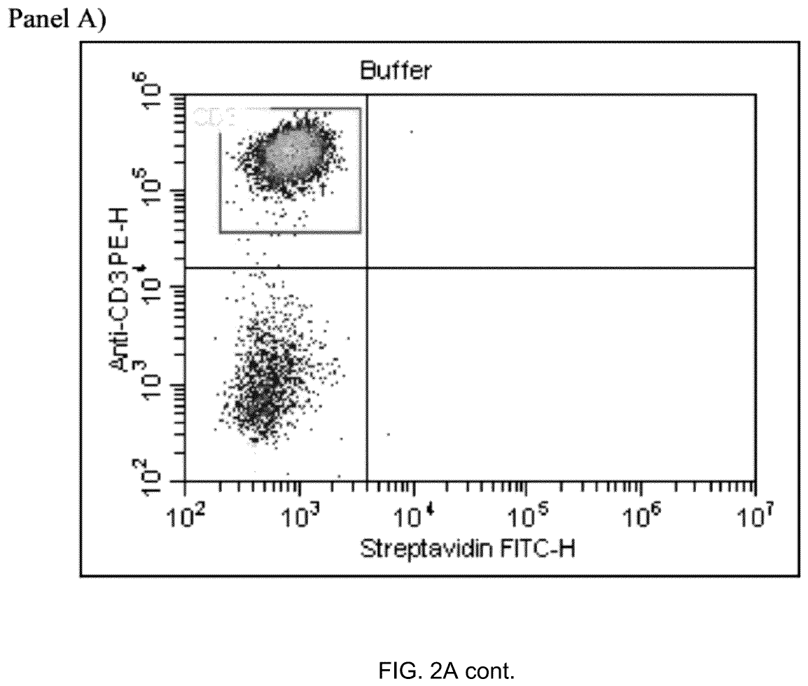

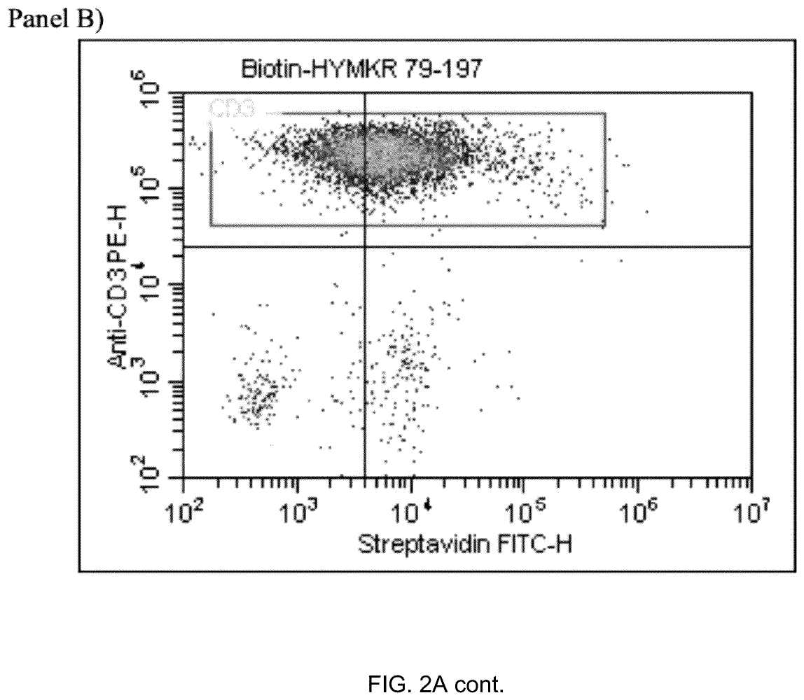

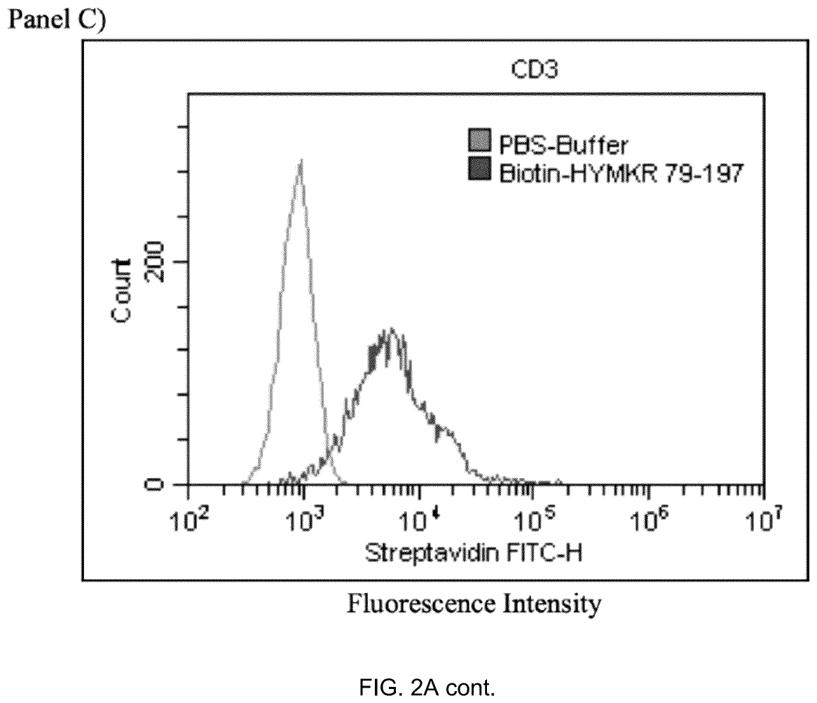

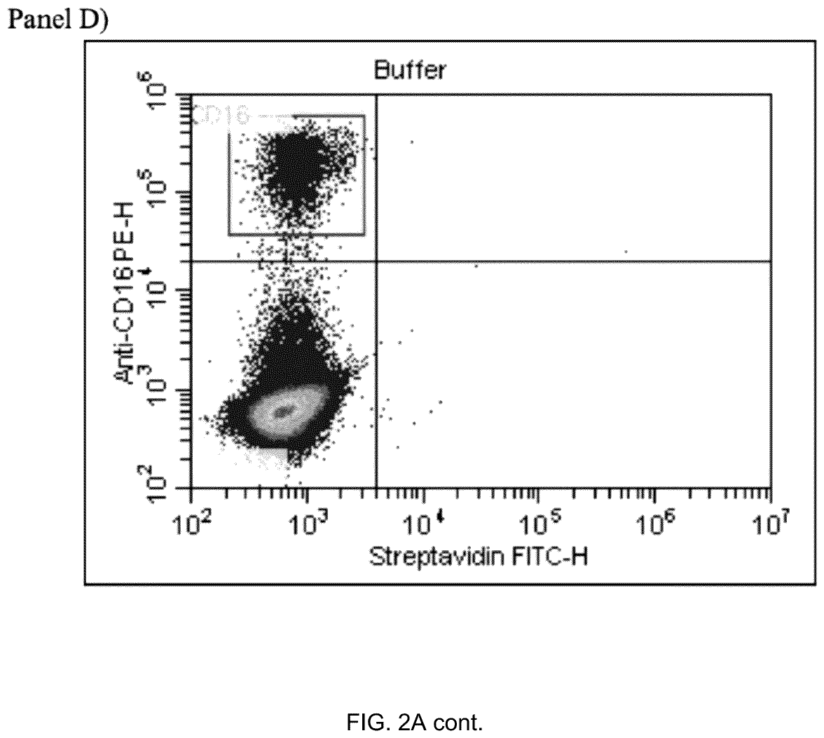

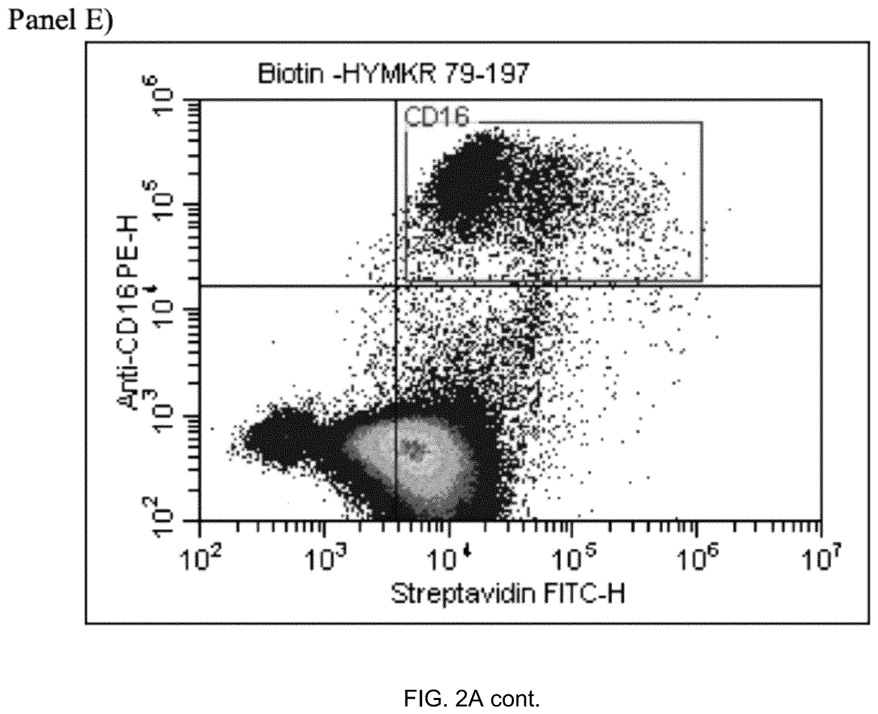

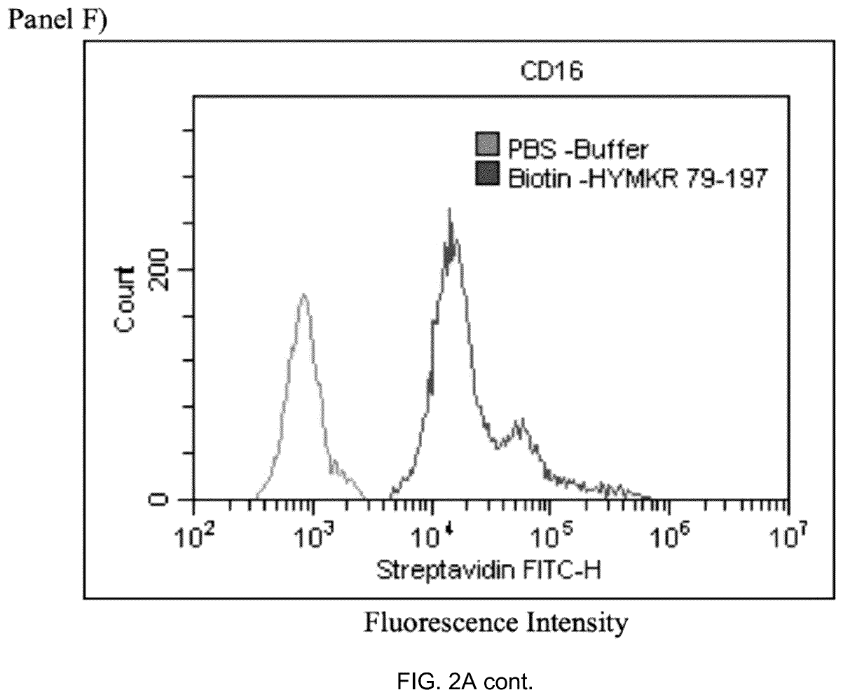

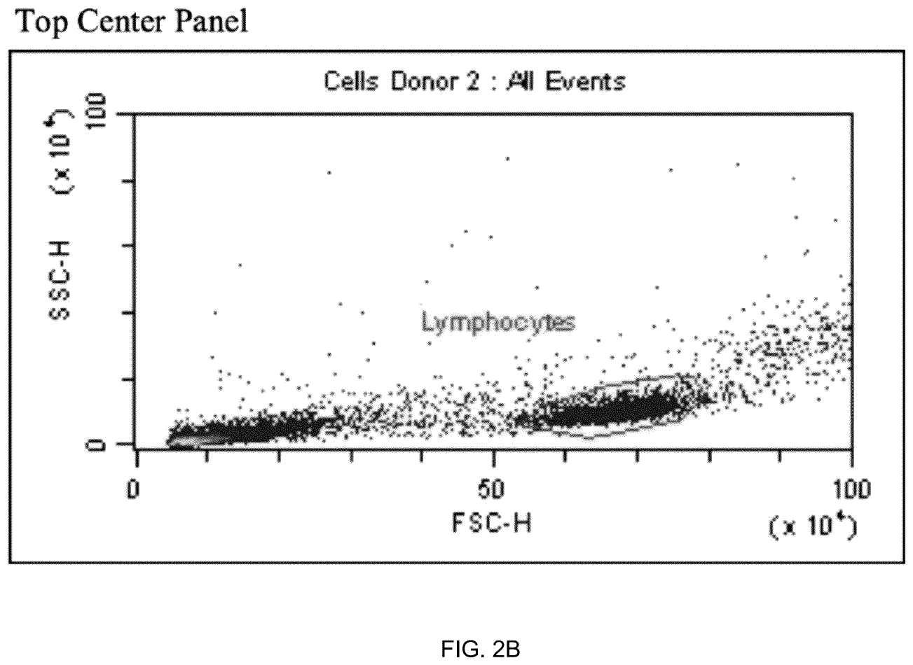

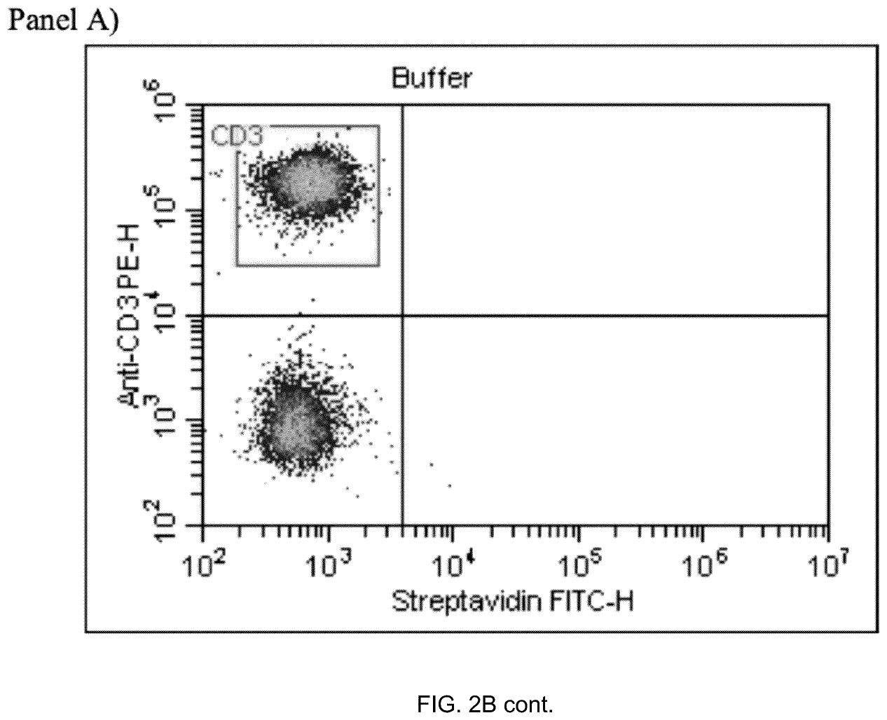

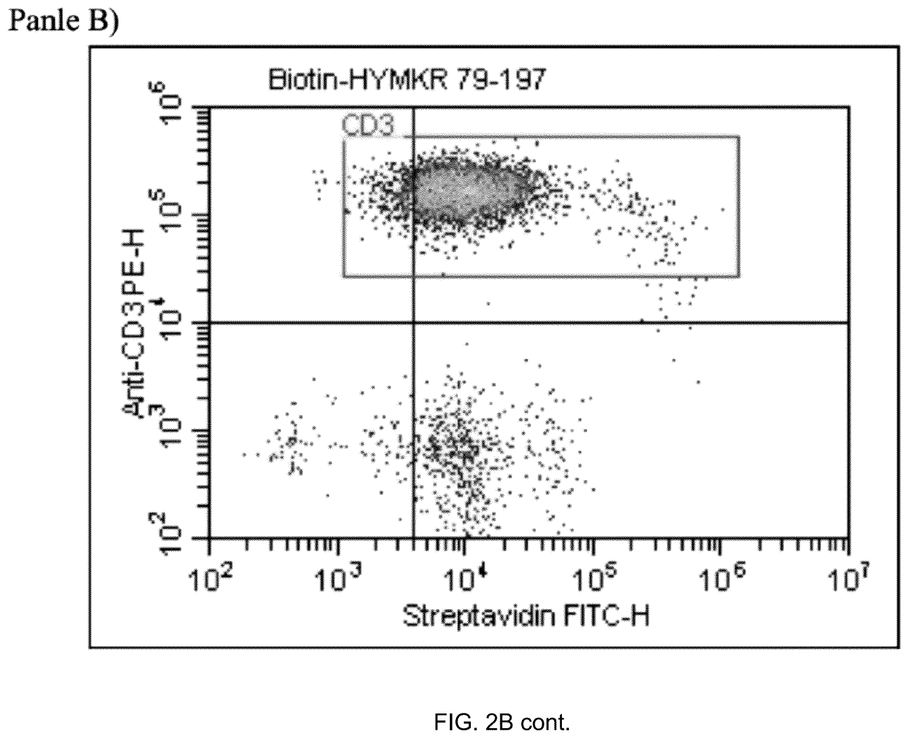

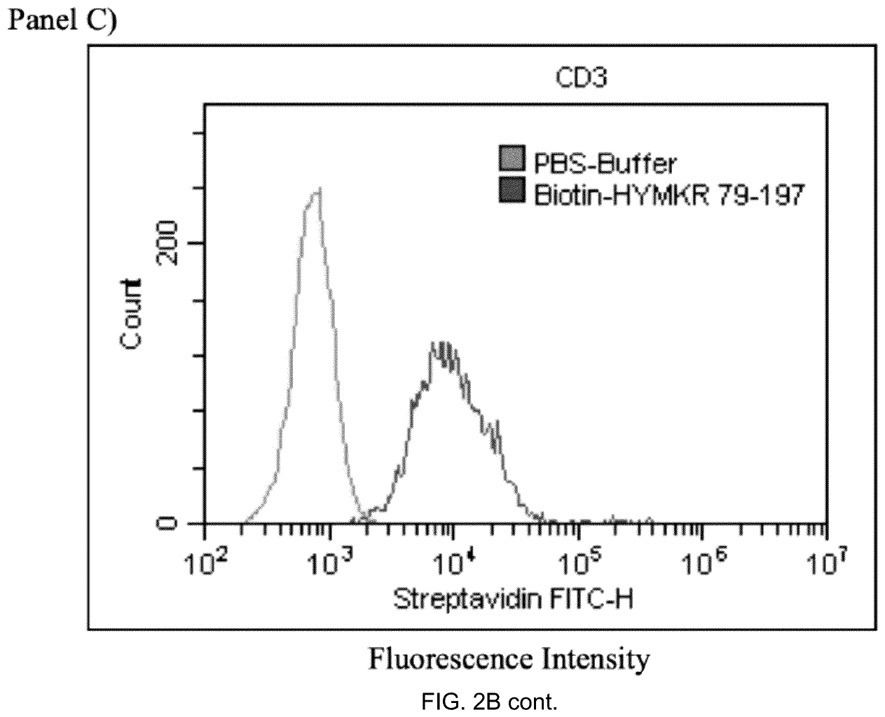

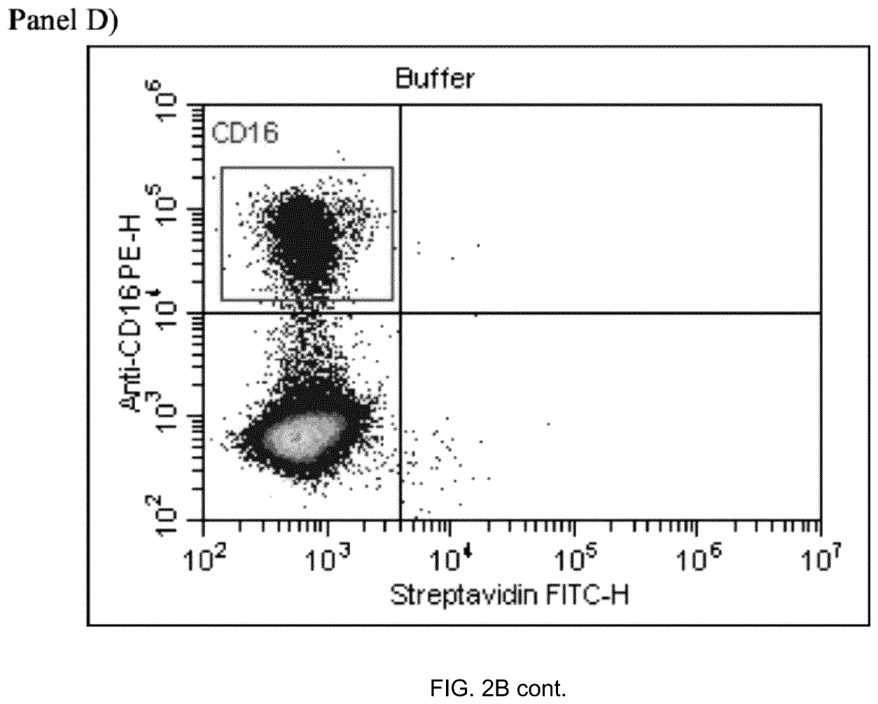

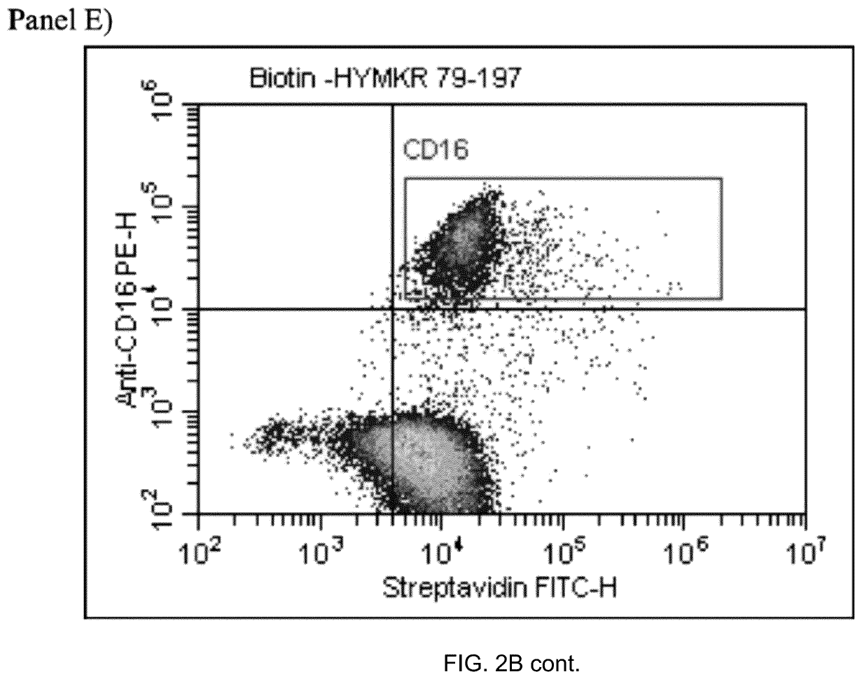

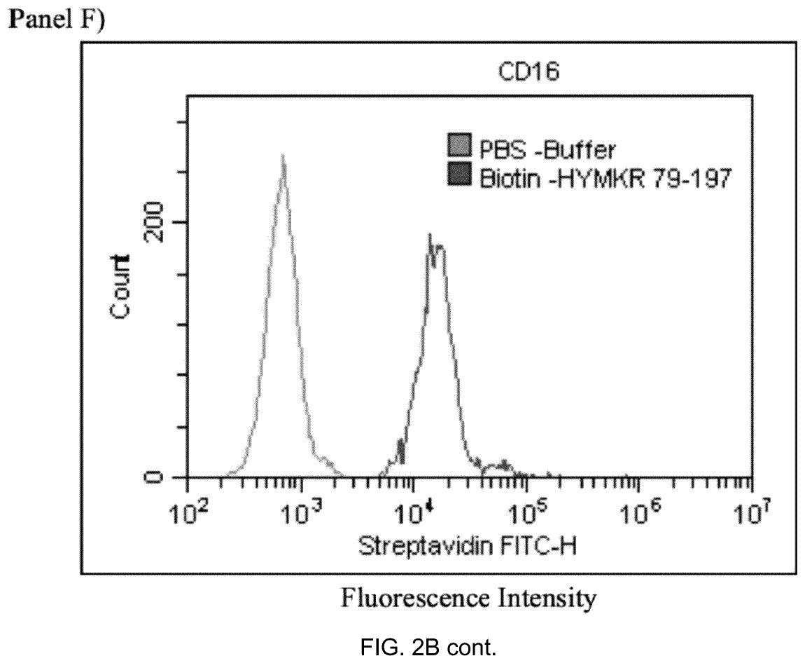

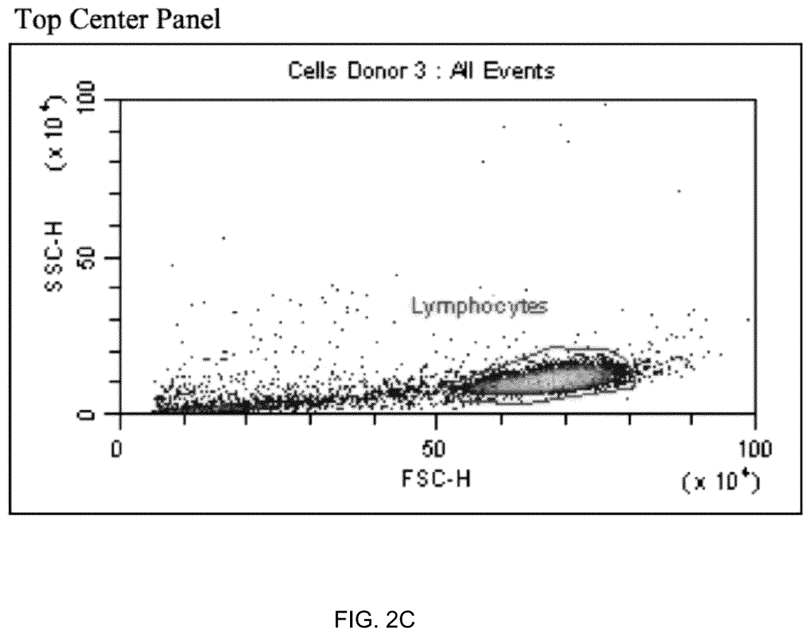

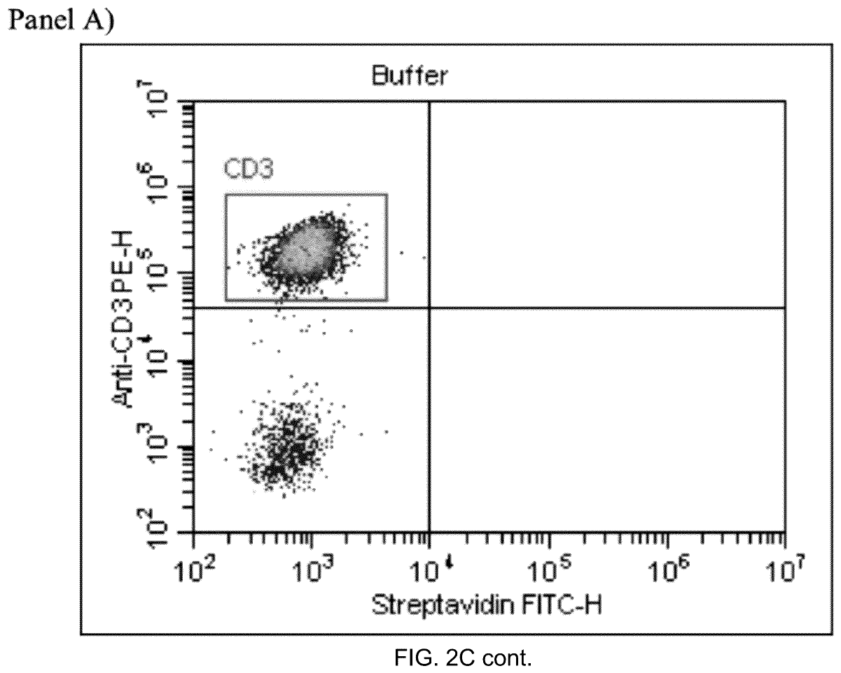

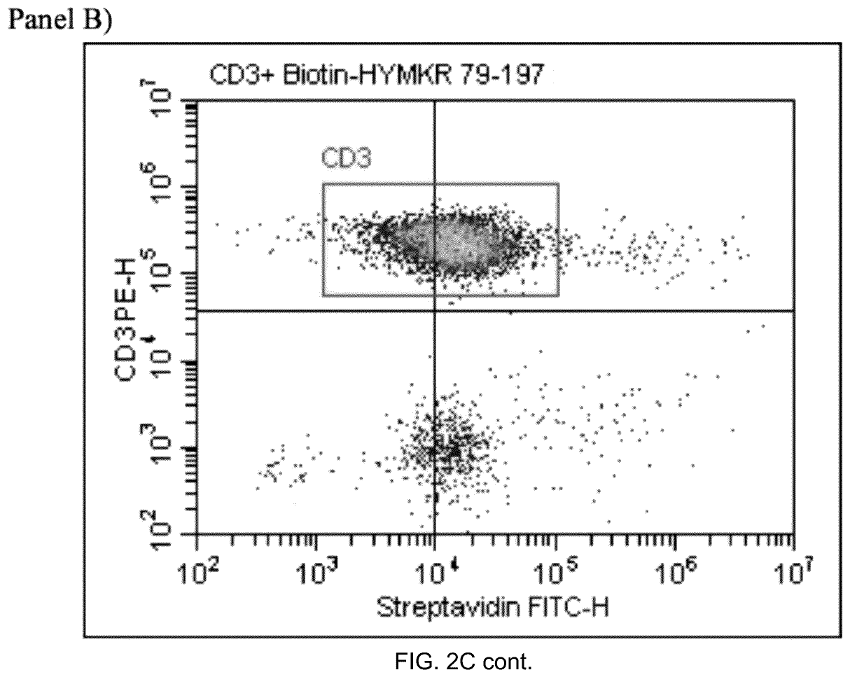

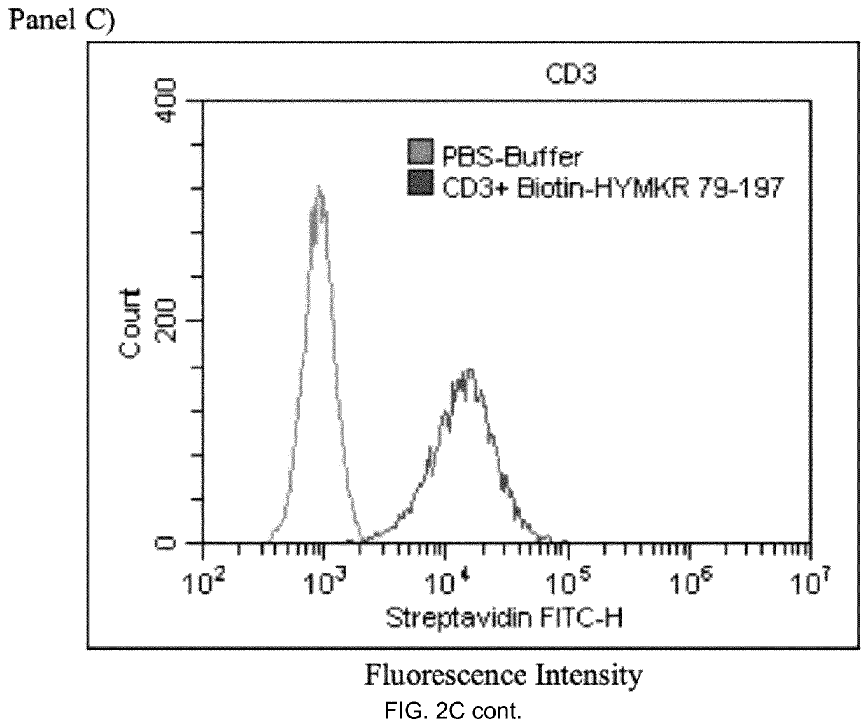

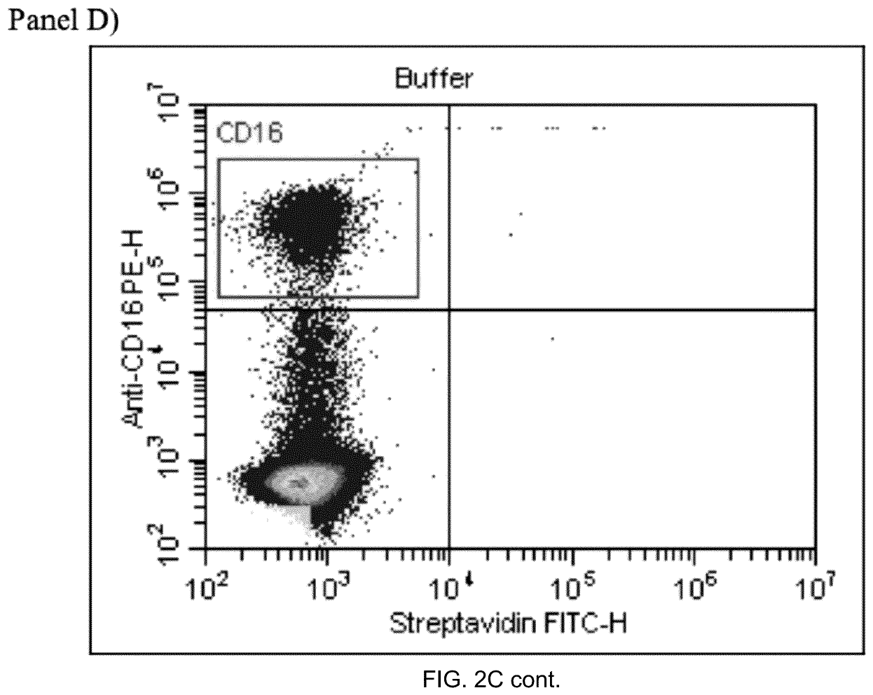

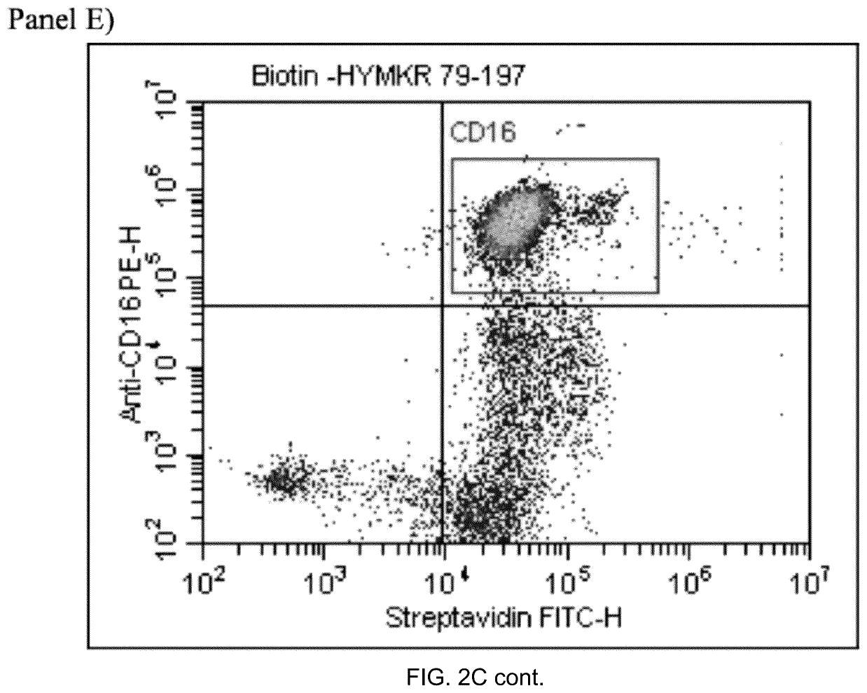

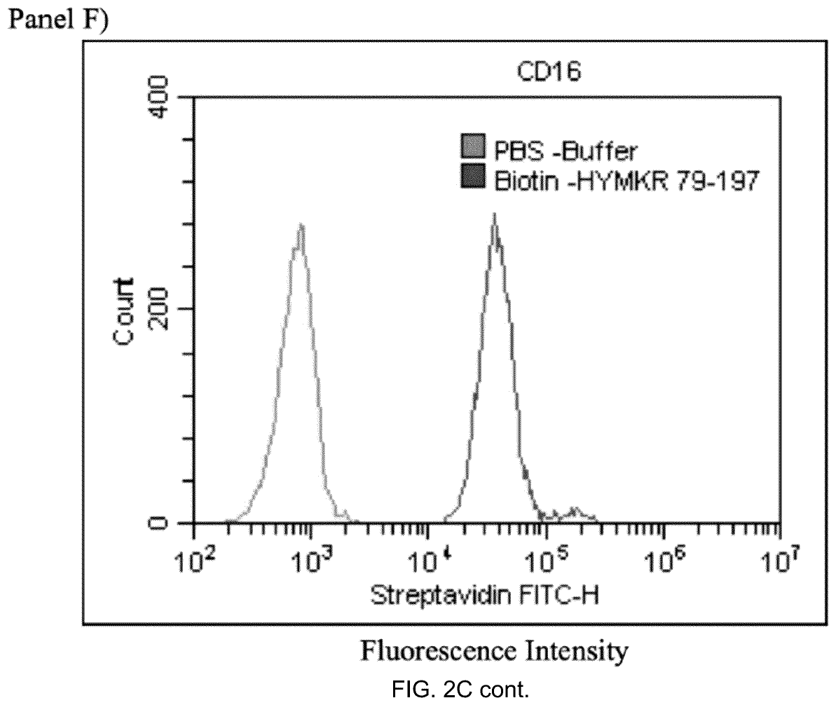

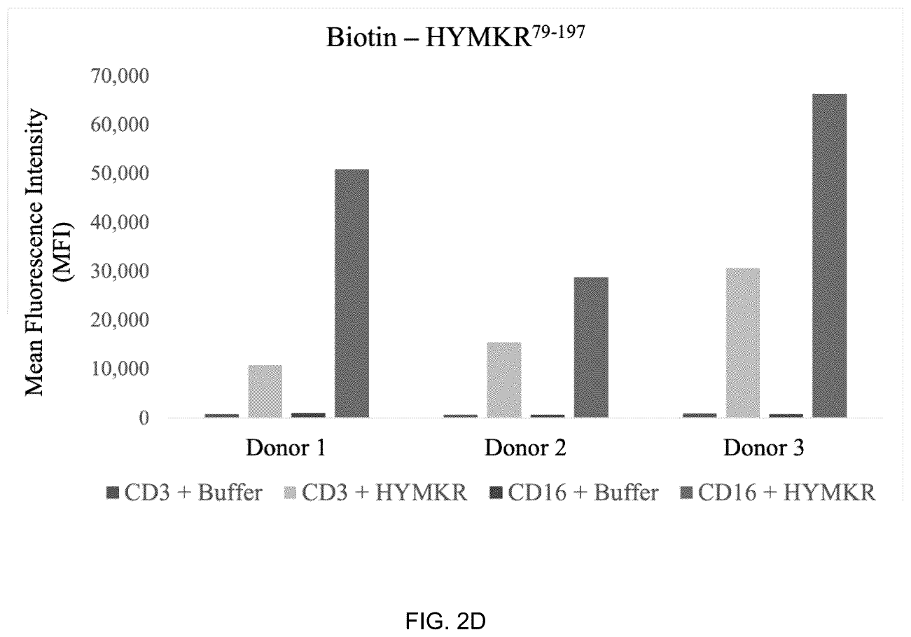

Flow cytometry assays were performed to determine whether HYMKR⁷⁹⁻¹⁹⁷ binds selectively to nNK cells relative to T cells. Studies from three different subjects are shown in FIGS. 2A, 2B and 2C. Biotinylated-HYMKR⁷⁹⁻¹⁹⁷ bound significantly greater to the surface of nNK cells relative to unstimulated T cells from three subjects (compare Panel C to Panel F of the three donors and FIG. 2D) These results suggest that HYMKR⁷⁹⁻¹⁹⁷ preferentially binds to the surface of NK cells via a putative nNK cell receptor.

More specifically, referring now to FIGS. 2A, 2B and 2C, biotinylated-HYMKR⁷⁹⁻¹⁹⁷ polypeptide interacts strongly with nNK cells compared to T cells. Flow cytometry was performed to examine the binding of biotinylated-HYMKR⁷⁹⁻¹⁹⁷ to CD3⁺ T cells and CD16⁺ NK cells of three healthy subjects, each of which are shown in FIGS. 2A, 2B and 2C, respectively. Freshly isolated peripheral blood lymphocytes were incubated with buffer as negative control (Panel A, T cells and Panel D, NK cells of each donor) or treated with 75 μg of biotinylated-HYMKR⁷⁹⁻¹⁹⁷(Panels B, T cells and Panel E, NK cells of each donor). Cells from the controls and experimental groups were probed with streptavidin-FITC to detect cell surface-bound biotinylated-HYMKR⁷⁹⁻¹⁹⁷. The treated cells were then reacted with anti-CD16 PE or anti-CD3 PE. Lymphocytes were first gated based on forward and side scatter (top center Panels) for downstream analyses with biotinylated-HYMKR⁷⁹⁻¹⁹⁷. HYMKR⁷⁹⁻¹⁹⁷ reacted strongly with CD16⁺ NK cells from each of the subjects (Panel E of each donor) but weakly with CD3⁺ T cells (Panel B of each donor). T cells from Panel B of each donor were gated and further analyzed in Panel C. NK cells from Panel E of each donor were gated and further analyzed in Panel F. A bar graph shows the MFI values of the gated negative controls and the gated biotinylated-HYMKR⁷⁹⁻¹⁹⁷ treated CD3⁺ cells and treated CD16⁺ lymphocytes (FIG. 2D). The average MFI of donor T cells (n=3) after the buffer control MFI was subtracted was 18,222±SD 10,311. The average MFI of donor NK cells (n=3) after subtraction of the buffer MFI was 47,862±SD 18,830. The null hypothesis: biotinylated-HYMKR⁷⁹⁻¹⁹⁷ reacts more strongly with CD16⁺ NK cells than CD3⁺ T cells due to the presence of its receptor (moesin) on the cell surface. Student's unpaired t-test (one-tailed), df=4, NK cells vs T cells, p=0.0376, significant at p≤0.05.

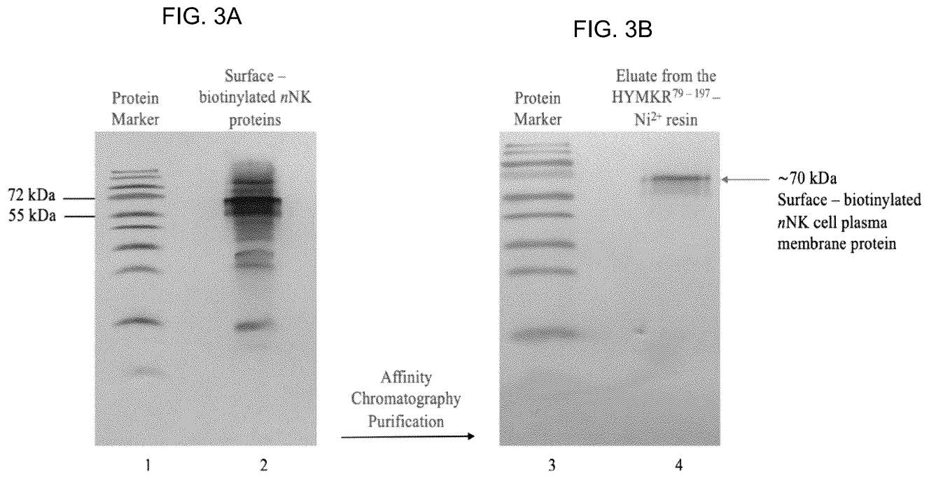

To detect and purify the nNK cell receptor for its ligand, HYMKR, experiments were performed in which the surface proteins of nNK cells were labelled with biotin. A lysate of surface-biotinylated negatively selected CD16⁺ NK cells (FIG. 3 Panel A) was applied to the HYMKR⁷⁹⁻¹⁹⁷-Ni²⁺ resin. The specifically bound proteins were eluted in a urea-imidazole buffer. A prominent biotinylated band was detected on western blots demonstrating that the molecular weight of the putative nNK cell receptor was approximately 70 kDa (FIG. 3 Panel B).

To determine the identity of the nNK cell receptor for HYMKR, we isolated the 60 to 72 kDa region of SDS-PAGE gels from the affinity purified negatively selected NK cells that presumably contained the nNK cell-HYMKR⁷⁹⁻¹⁹⁷ binding protein (as seen in FIG. 3B).

More specifically, FIGS. 3A and 3B depict affinity chromatography purification of surface biotinylated proteins of NK cells from HYMKR⁷⁹⁻¹⁹⁷-Ni²⁺ resin. Freshly isolated nNK cells from peripheral blood were enriched about 38-fold (2.5% to 95%) by negative selection and then surface biotinylated as previously described (See e.g., Das, B., et al., Preferential interaction of a novel tumor surface protein (p38.5) with naive natural killer cells. J Exp Med, 1997. 185(10): p. 1735-42). FIG. 3A depicts Western blot of surface-biotinylated nNK cell plasma membrane proteins prior to affinity chromatography showing numerous proteins. FIG. 3B depicts Western blot of surface-biotinylated nNK plasma membrane proteins after affinity chromatography purification on a HYMKR⁷⁹⁻¹⁹⁷-Ni²⁺ resin via a 6×His-tag. SDS-PAGE and Western blot of the biotinylated nNK cell surface proteins were probed with streptavidin-alkaline phosphatase. A single band of approximately 70 kDa was detected in the eluate from the HYMKR⁷⁹⁻¹⁹⁷-Ni²⁺ resin.

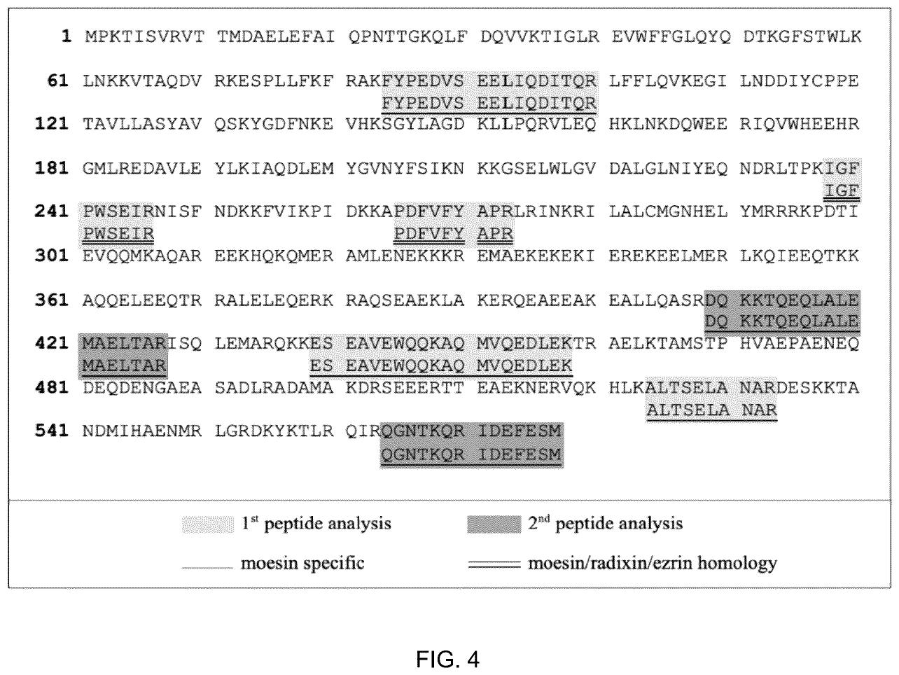

The protein extracted from the gel was treated with proteases, and the resulting peptides were sequenced as described in Materials and Methods (See also, FIG. 4).

More specifically, FIG. 4 relates to two experimental approaches to identify the plasma membrane surface protein on nNK cells that binds to the HYMKR⁷⁹⁻¹⁹⁷-Ni²⁺ resin. Amino acid sequences of peptides obtained from two separate gel slices (between 60 to 72 kDa) of proteins that were eluted from the HYMKR⁷⁹⁻¹⁹⁷-Ni²⁺ resin. Amino acid sequences of these peptides were determined by mass spectrometry (Harvard University Center for Mass Spectrometry Proteomics Portal, Cambridge, MA). In the first experiment a standard approach was used where the eluate from the HYMKR⁷⁹⁻¹⁹⁷-Ni²⁺ resin was applied to an SDS PAGE gel and the 60 to 72 kDa region recovered and sent for amino acid sequencing. This procedure resulted in more than 38 protein candidates. In order to reduce the number of candidate proteins a second procedure was used where the purified nNK cells were first surface biotinylated before affinity chromatography, SDS PAGE and amino acid sequence analysis. This procedure reduced the number of proteins identified as candidate nNK cell receptors to three. These three proteins were also identified in the first experiment. The sequences of these peptides were identical to the sequence of moesin (GenBank accession no. NG_012516). The first peptide digest from non-biotinylated surface proteins, shown in blue, revealed three peptides specific to moesin (single-underlined) and two peptides that shared homology with radixin and ezrin (double-underlined). The second peptide digest, which included surface biotinylated proteins, as well as non-biotinylated proteins, shown in orange, revealed two moesin specific sequences. This was the only plasma membrane protein of the three seen in both experiments.

Peptide analysis from the initial experiment revealed 38 candidate proteins with a molecular weight of 65 to 75 kDa (data not shown). In an effort to reduce the number of possible NK cell receptor protein candidates a second experiment using a different technique was performed. The surface of nNK cells was biotinylated and then applied to the HYMKR⁷⁹⁻¹⁹⁷-Ni²⁺ resin along with the non-biotinylated extract. The specifically bound protein(s) from this extract were eluted and processed on an SDS-PAGE. A gel slice between 60 to 72 kDa was obtained and the peptides were sequenced. Analysis of the second experiment showed a different distribution and fewer peptides. Only three candidate molecules instead of 38 between 65 to 75 kDa were identified in this latter experiment: moesin, lamin-B1, and heat shock cognate 71 kDa protein. Significantly, these three proteins were also found in the list of 38 proteins from the first peptide digest thereby reducing the number of candidate molecules to investigate.

Subsequent focus on moesin was based on previous studies suggesting a possible role of ezrin, radixin, and moesin (ERM, cytoskeleton adaptor family proteins) in NK cell function (See e.g., Ramoni, C., et al., Differential expression and distribution of ezrin, radixin and moesin in human natural killer cells. Eur J Immunol, 2002. 32(11): p. 3059-65). Peptides of moesin from the first analysis are shown in FIG. 4. Two peptides share sequence homology between moesin (GenBank accession no. NG_012516), radixin (GenBank accession no. NG_023044), and ezrin (GenBank accession no. NG_052952). While radixin and ezrin could not be completely ruled out on this basis, three other peptides were unique to moesin. Moreover, the two peptides from the second experiment were unique to moesin (FIG. 4).

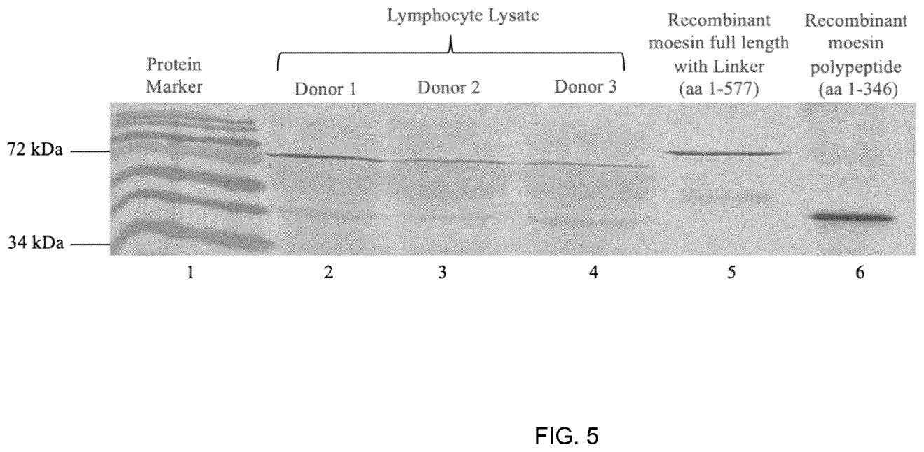

Accordingly, moesin is expressed on the exterior of the nNK plasma membrane since biotinylated-HYMKR⁷⁹⁻¹⁹⁷ bound to non-permeabilized CD16⁺ cells as shown in FIGS. 2A, 2B and 2C. In order to verify that moesin is differentially expressed on the exterior of the plasma membrane of nNK cells as compared to T cells, affinity chromatography purified anti-moesin rabbit antibody was used to demonstrate cell surface moesin expression. The specificity of this antibody is shown in FIG. 5.

More specifically, FIG. 5 depicts mono-specificity of affinity purified rabbit anti-moesin antibody. The anti-moesin rabbit polyclonal antibody (immunogen, amino acids 1 to 294) was purified by the manufacturer by affinity chromatography on an immunogen column. This antibody reacted with recombinant moesin polypeptide of 42 kDa, (the immunogen, Sino Biological Inc, #13659-H07E), a 92 kDa (full length protein linked to GST (Novus Biologicals, #H00004478-P01) and a 70 kDa band from lymphocyte extracts of three subjects.

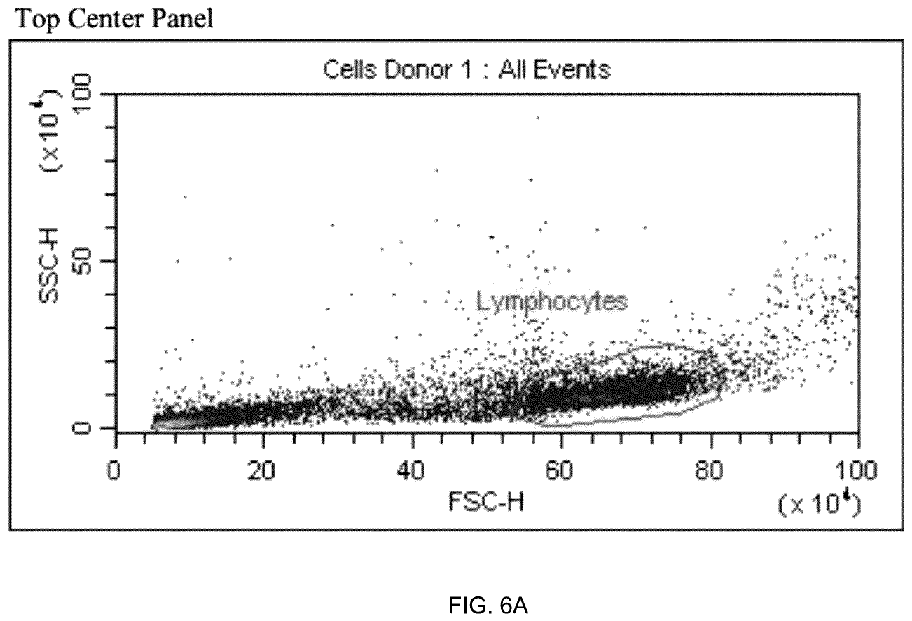

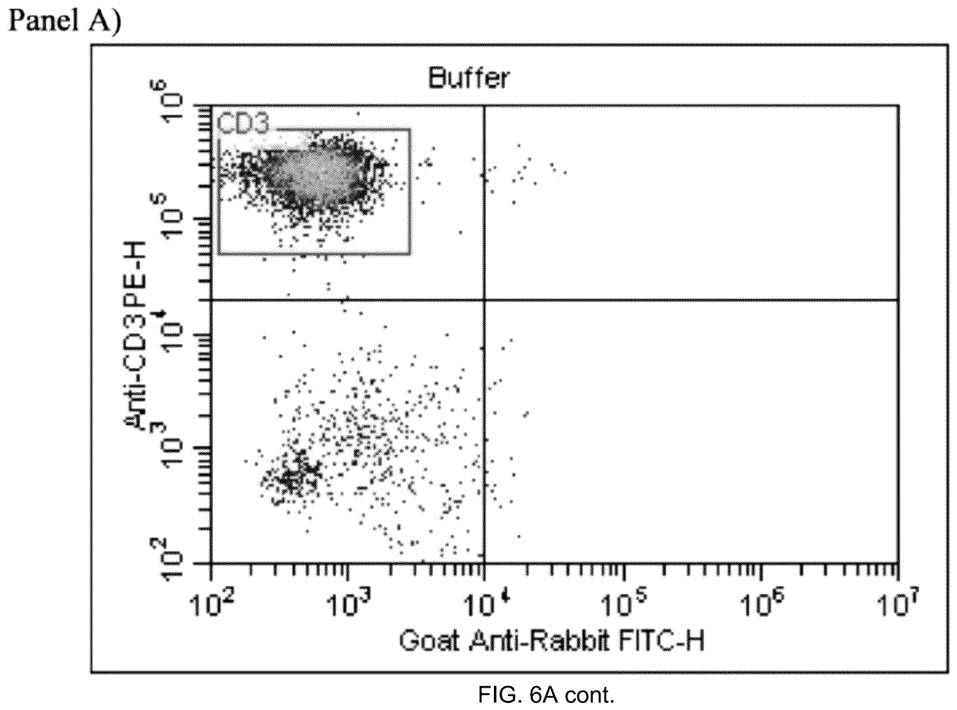

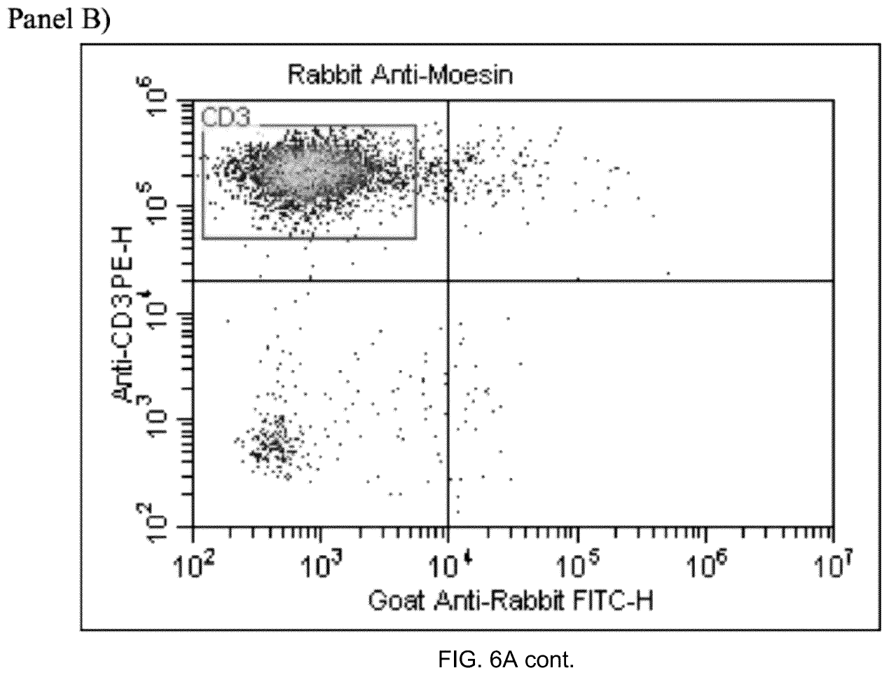

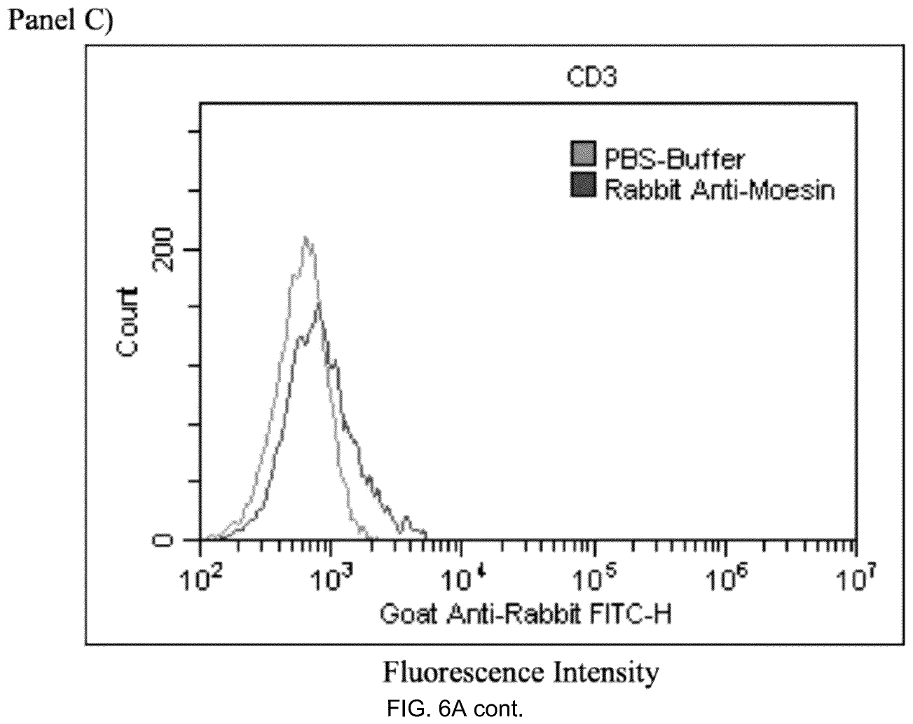

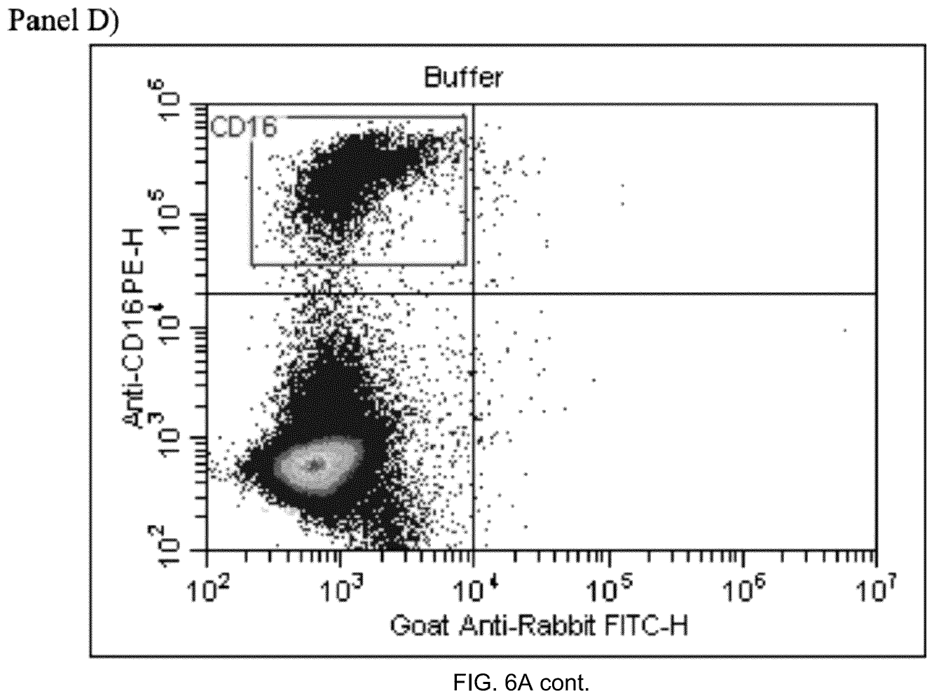

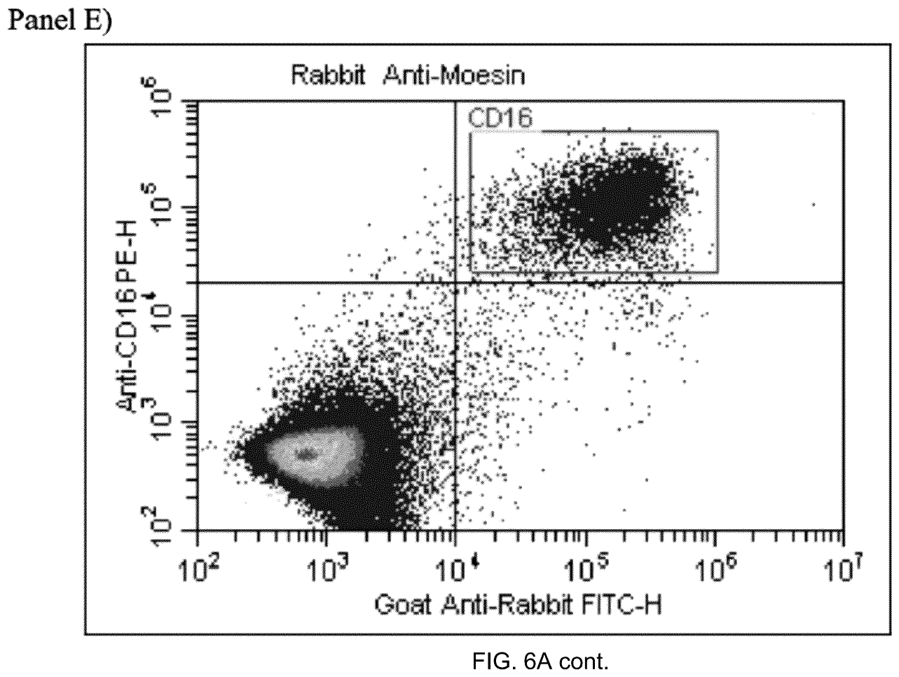

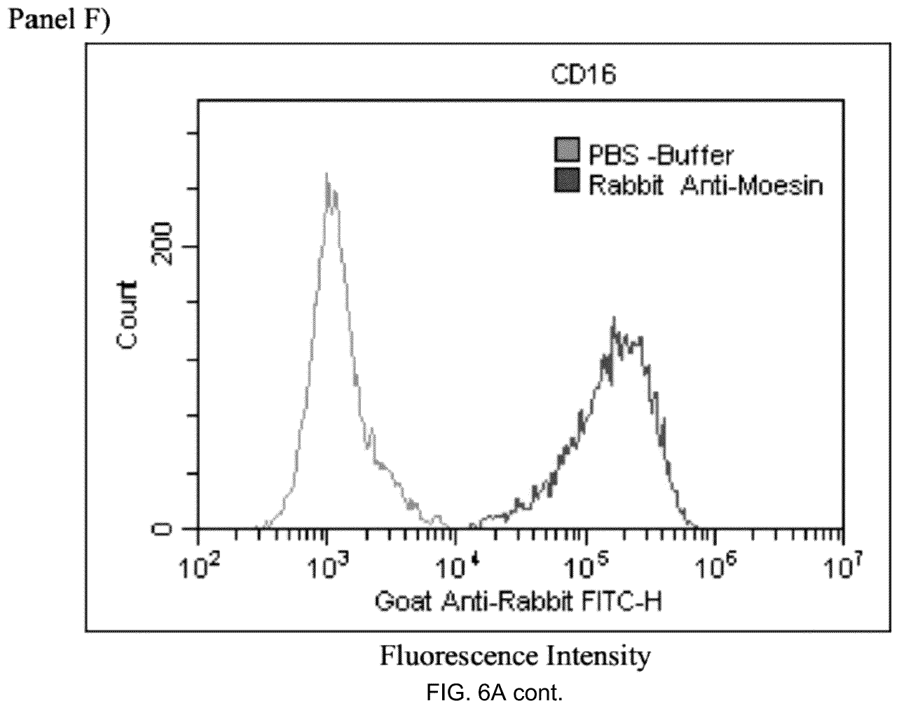

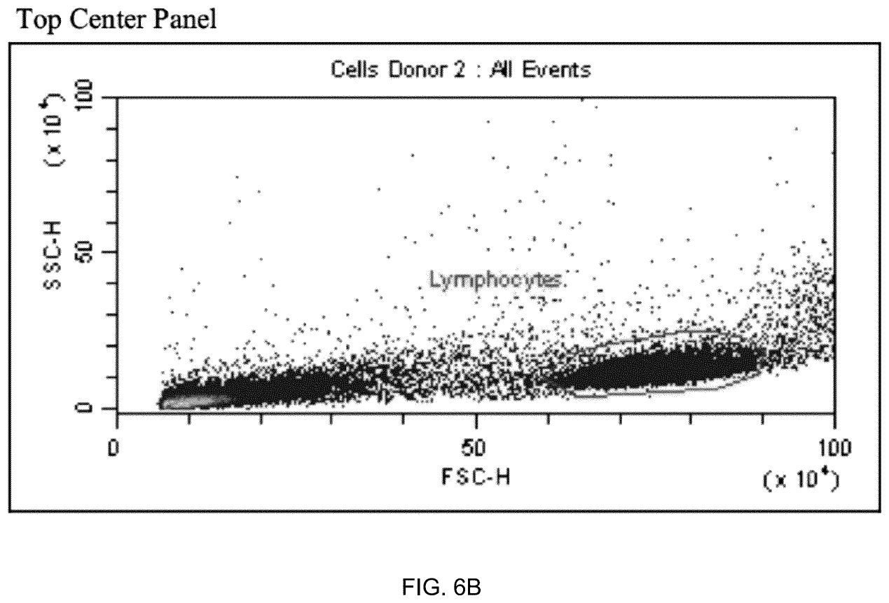

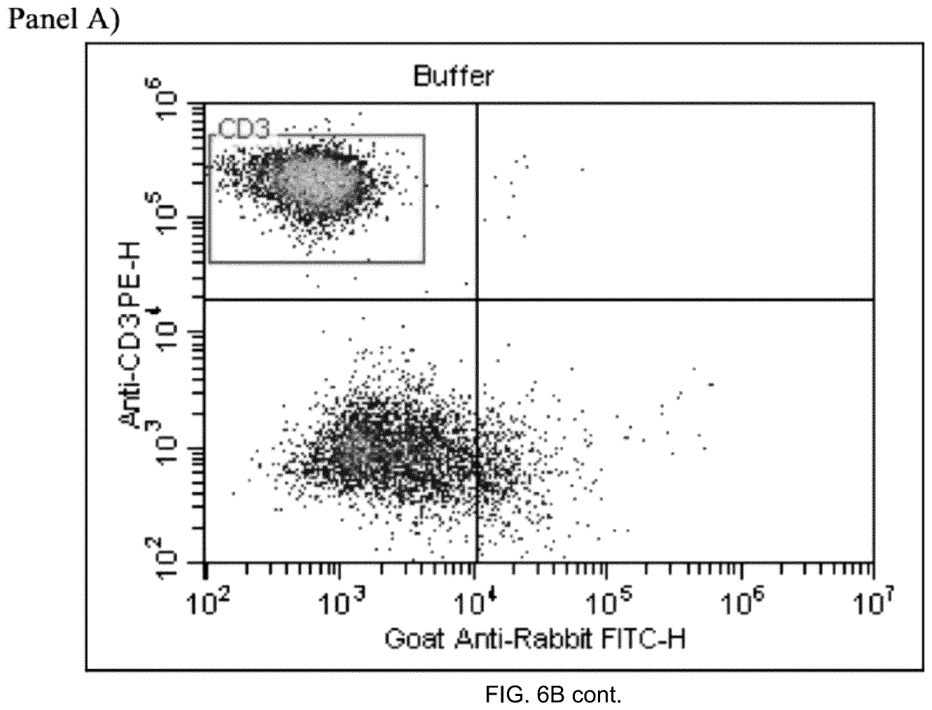

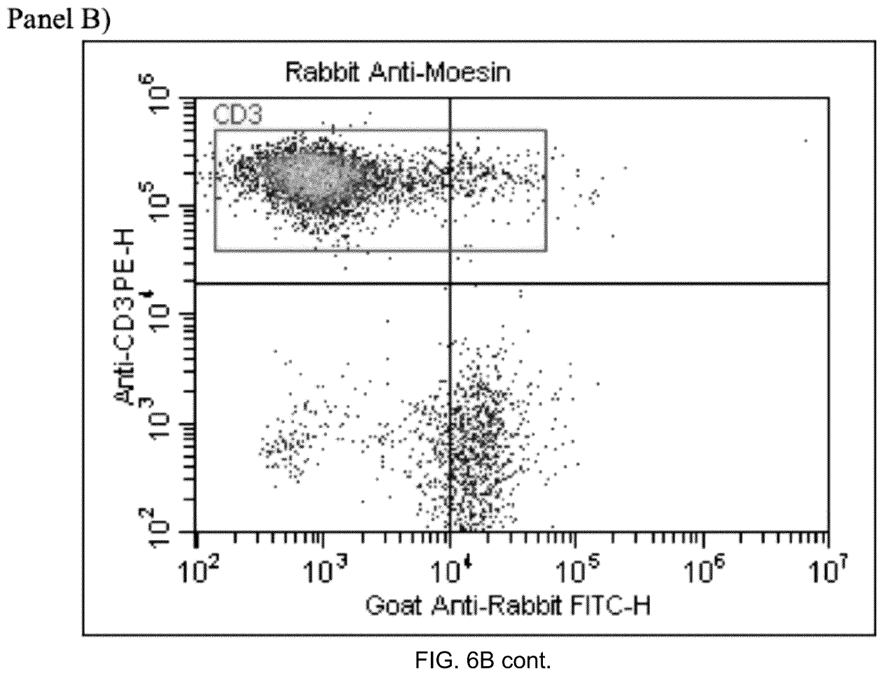

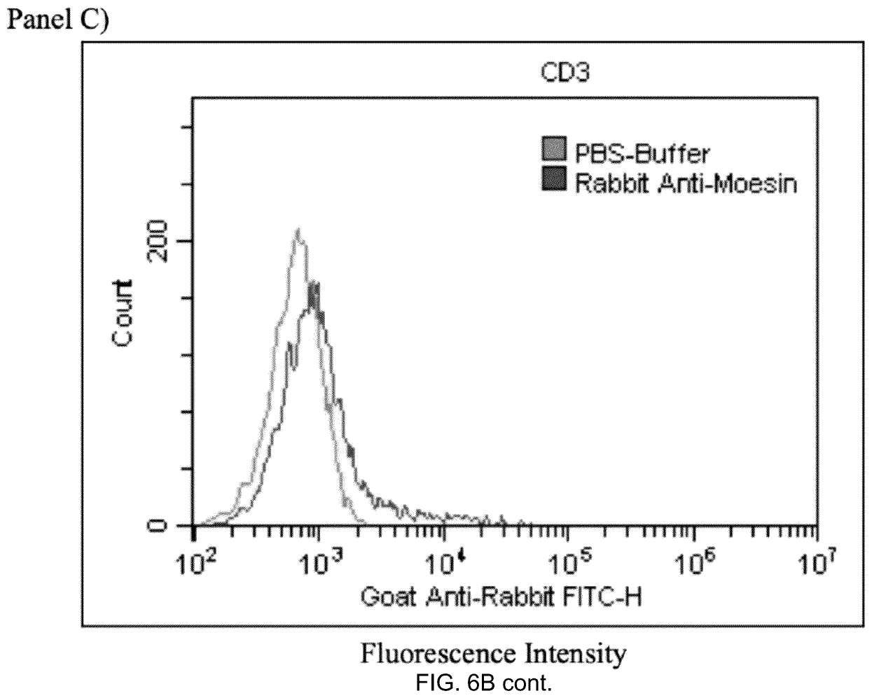

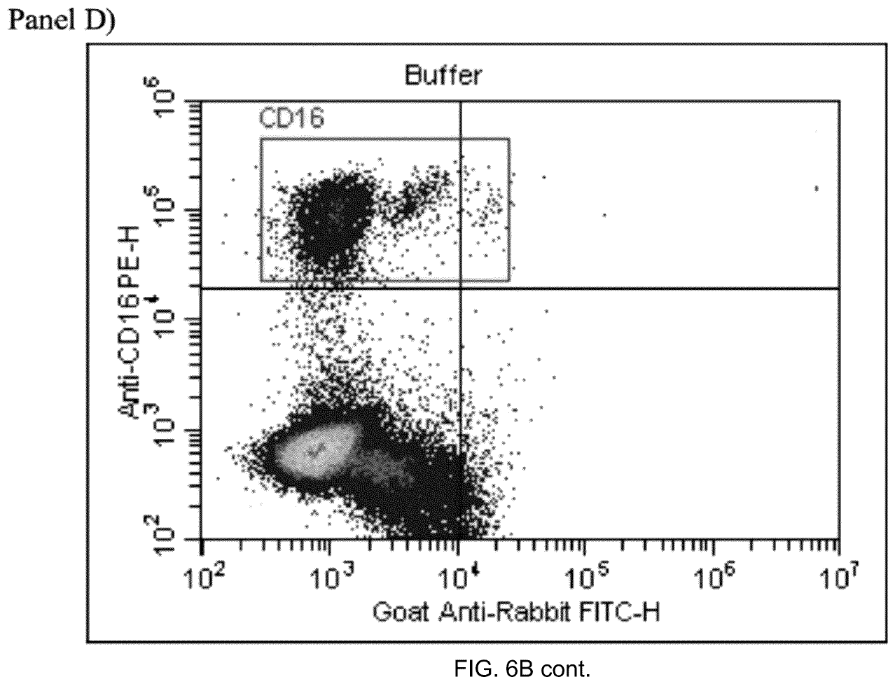

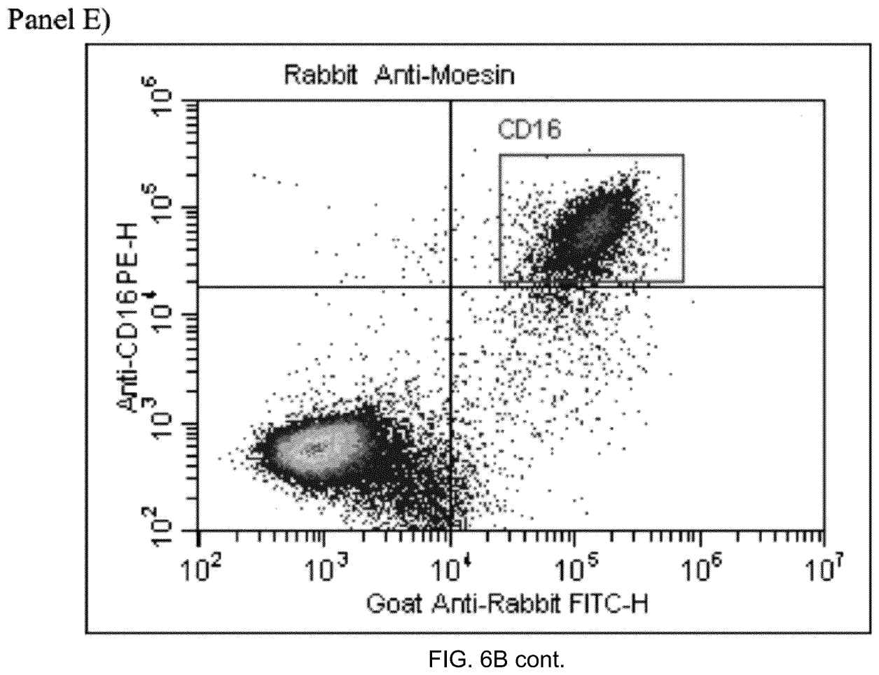

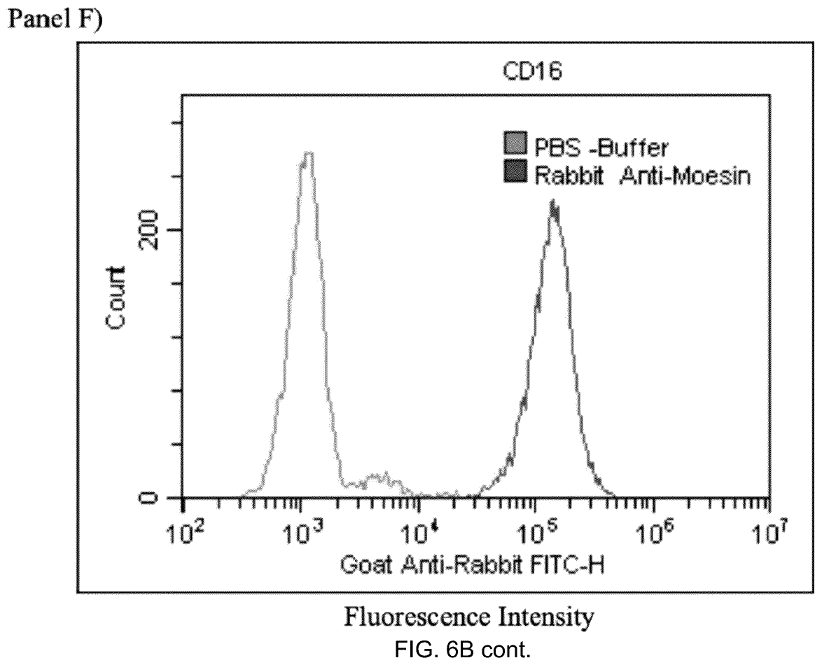

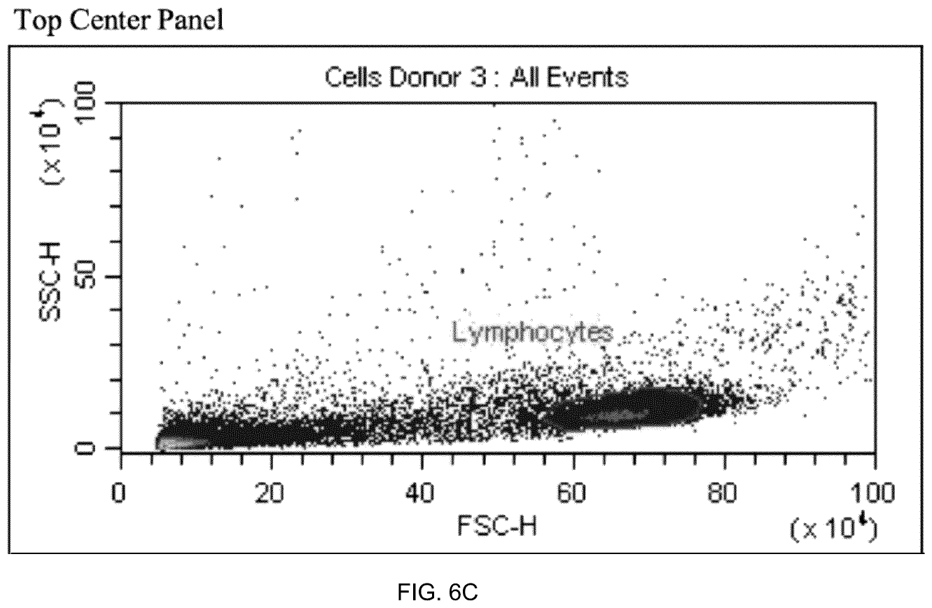

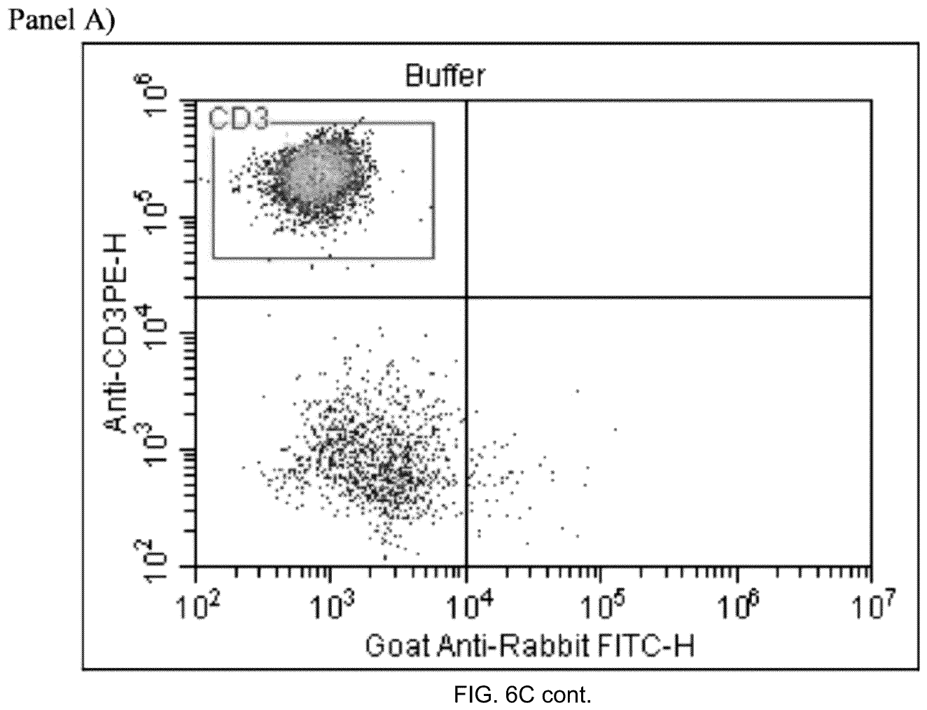

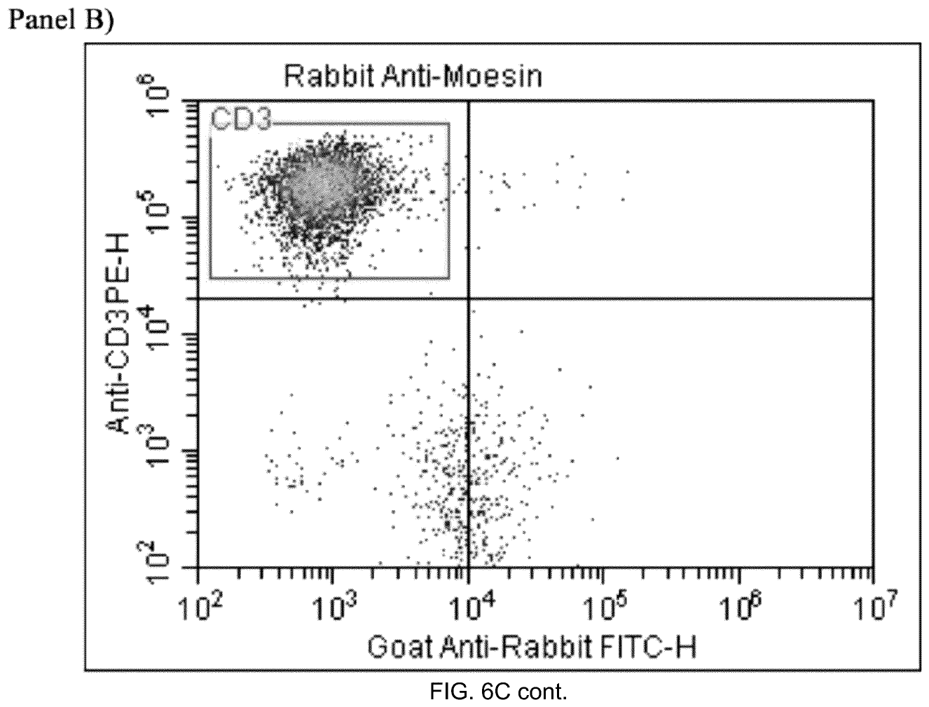

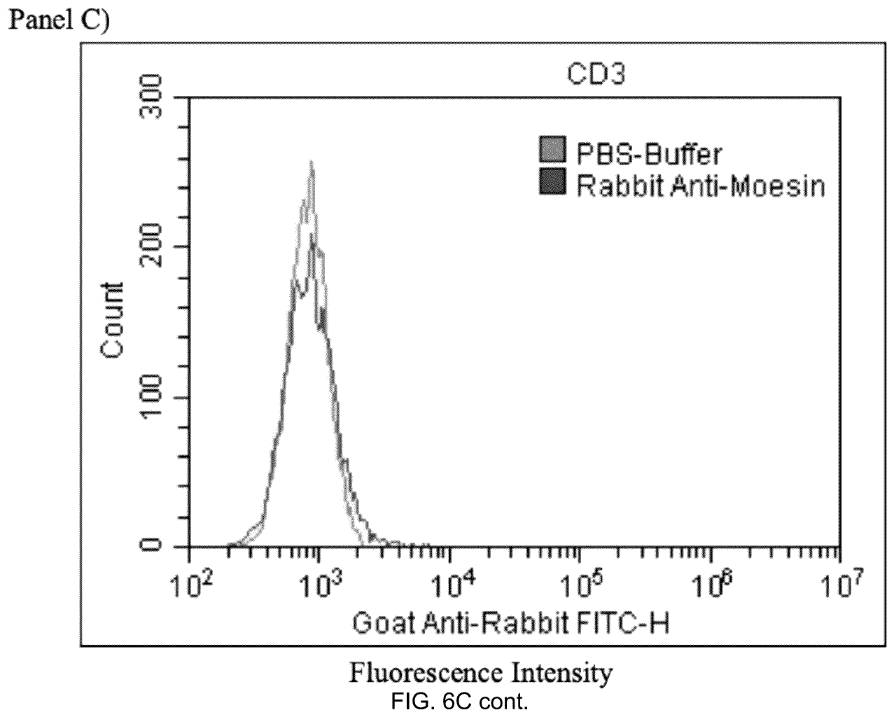

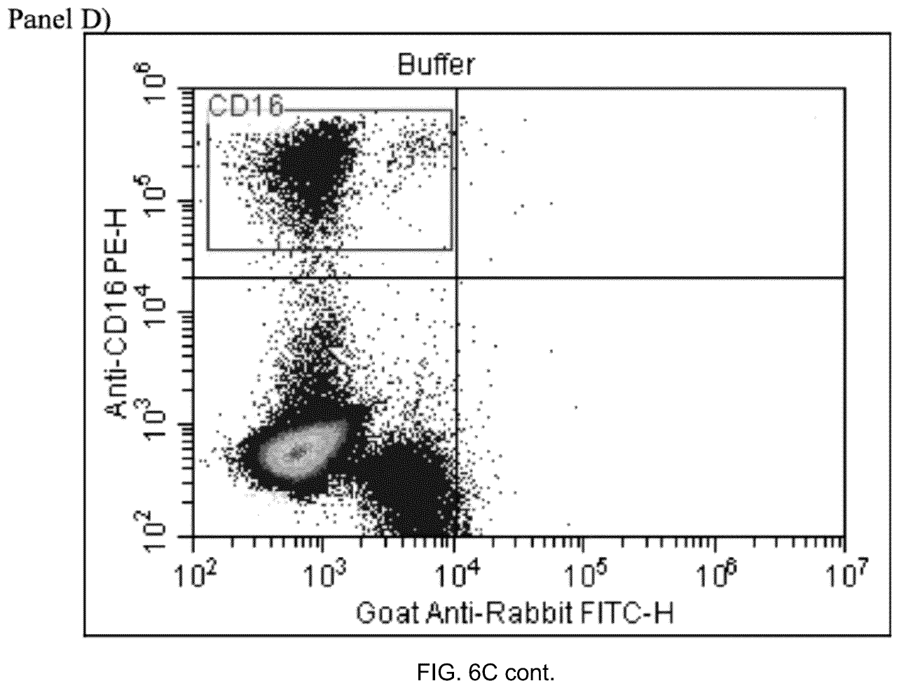

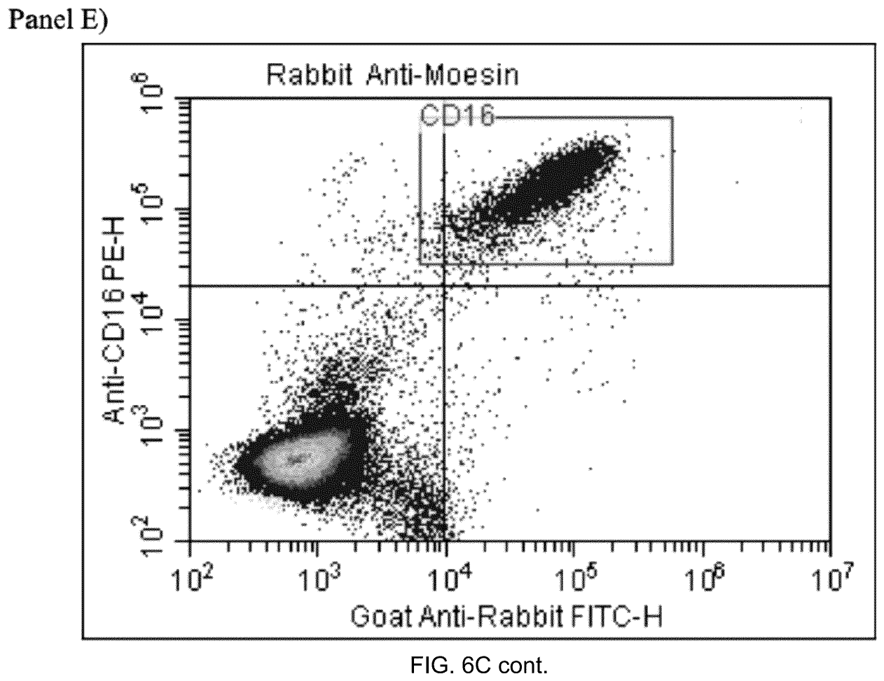

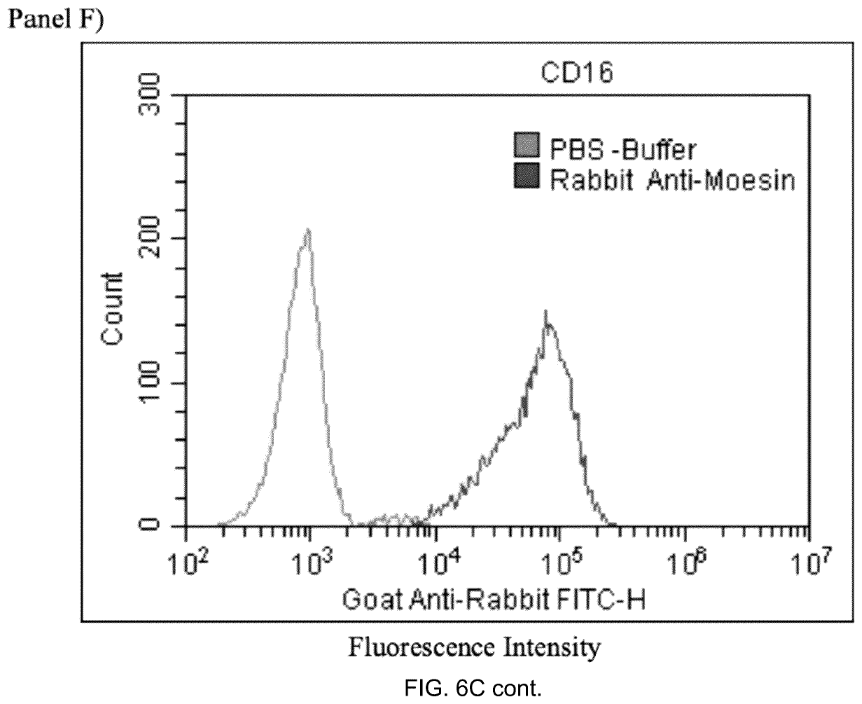

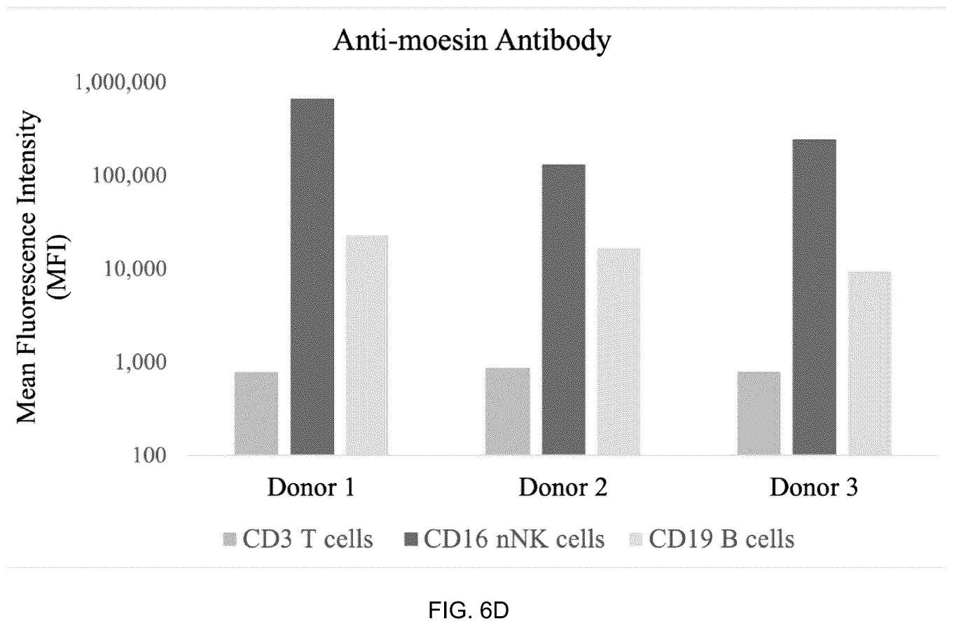

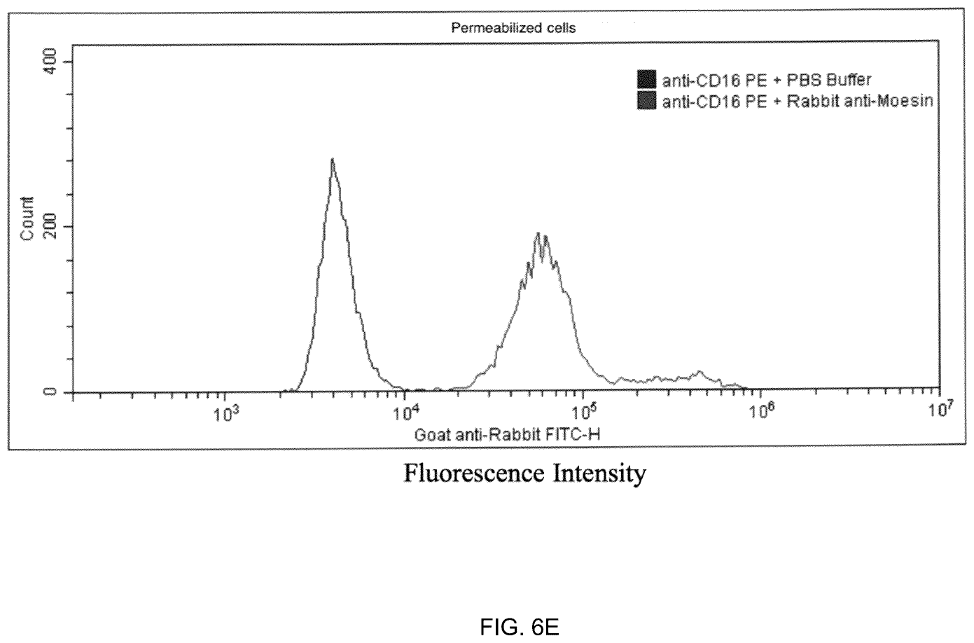

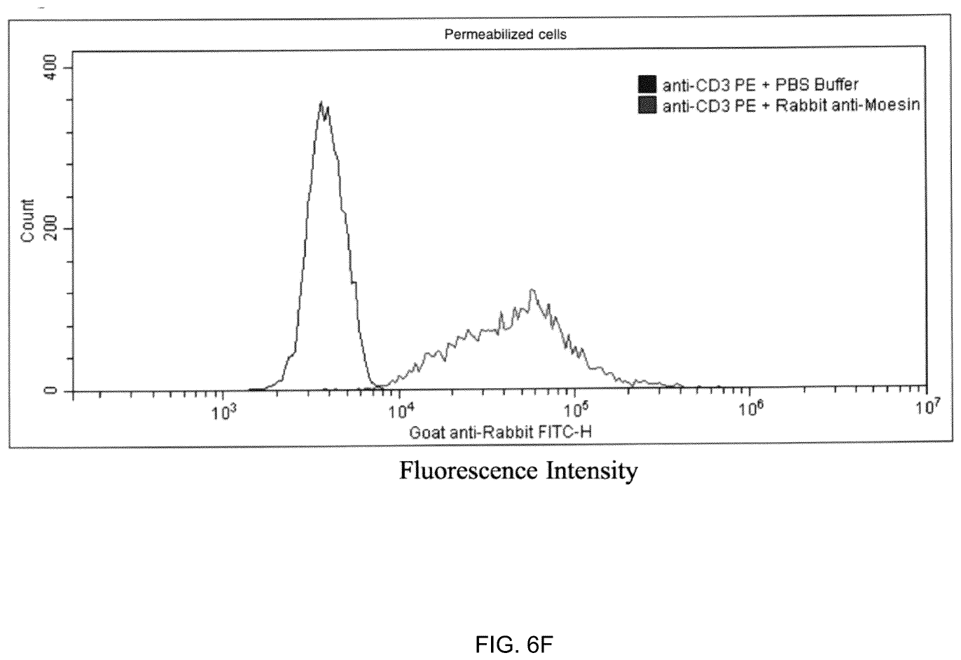

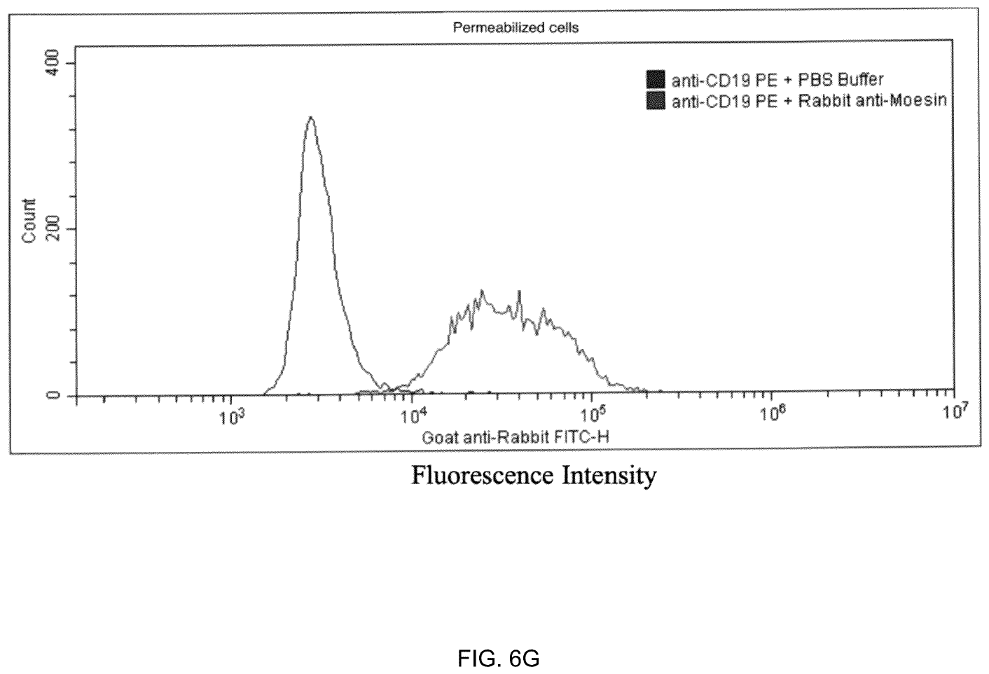

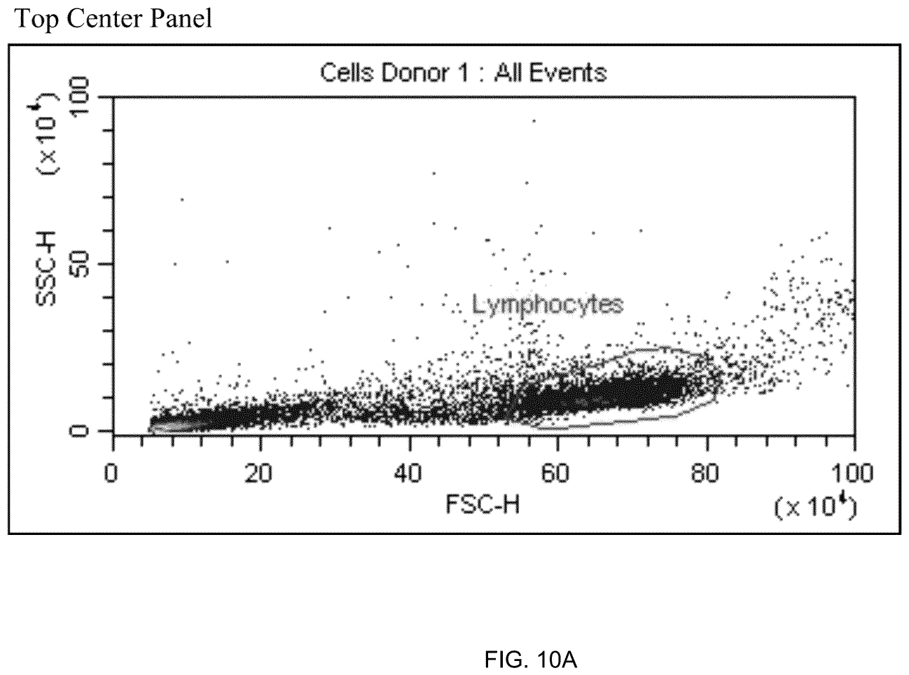

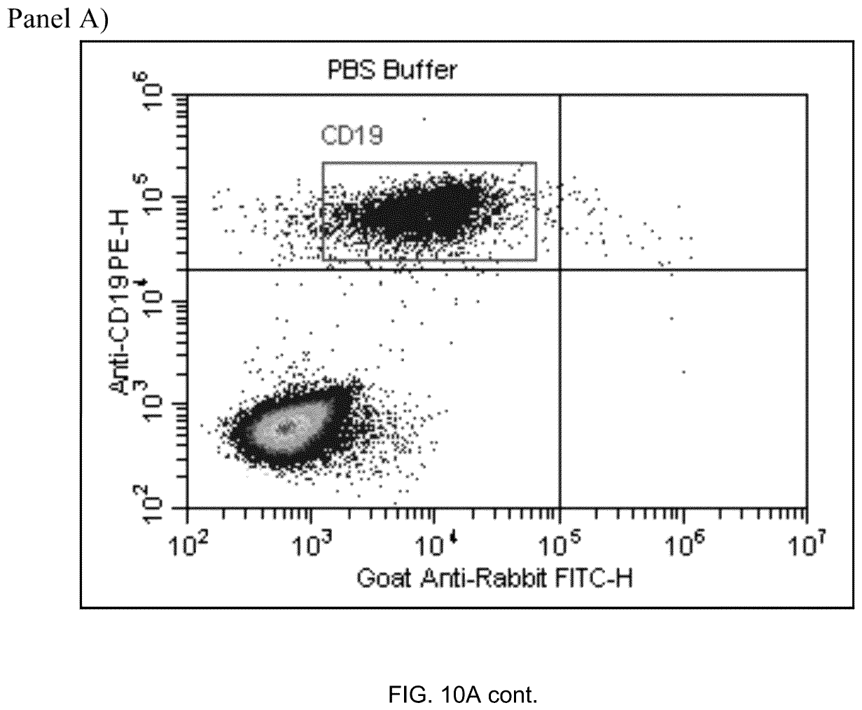

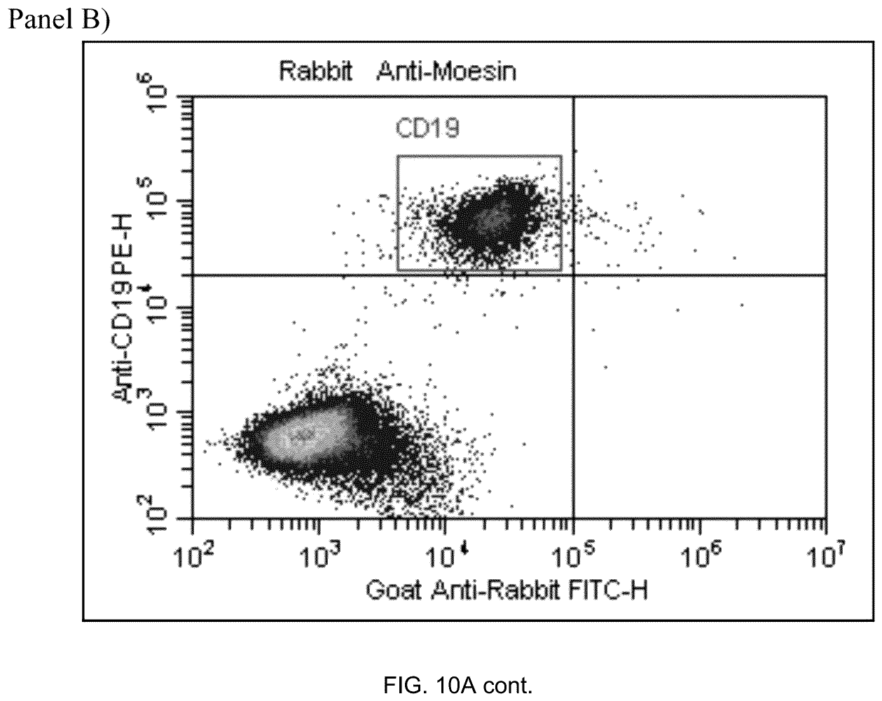

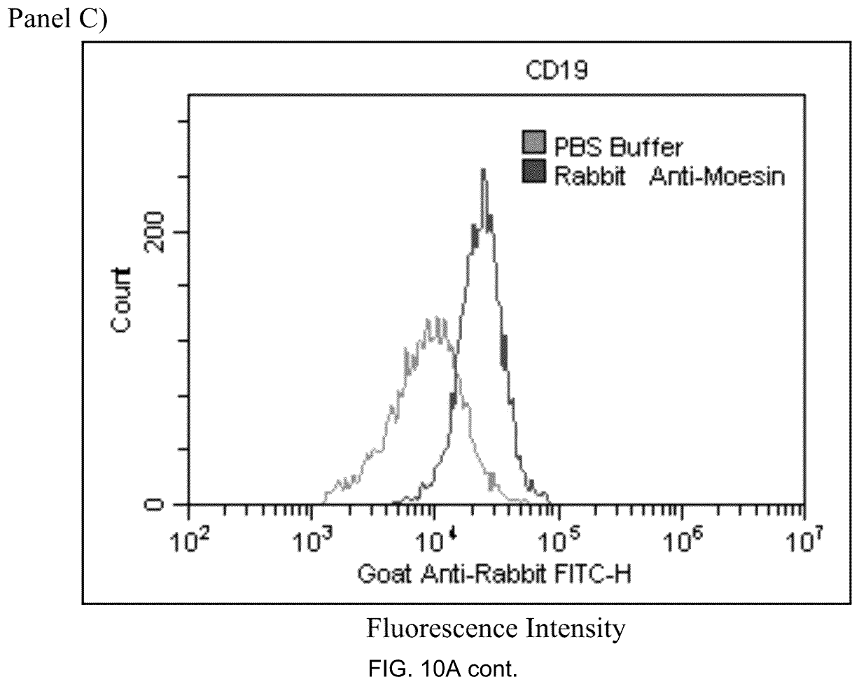

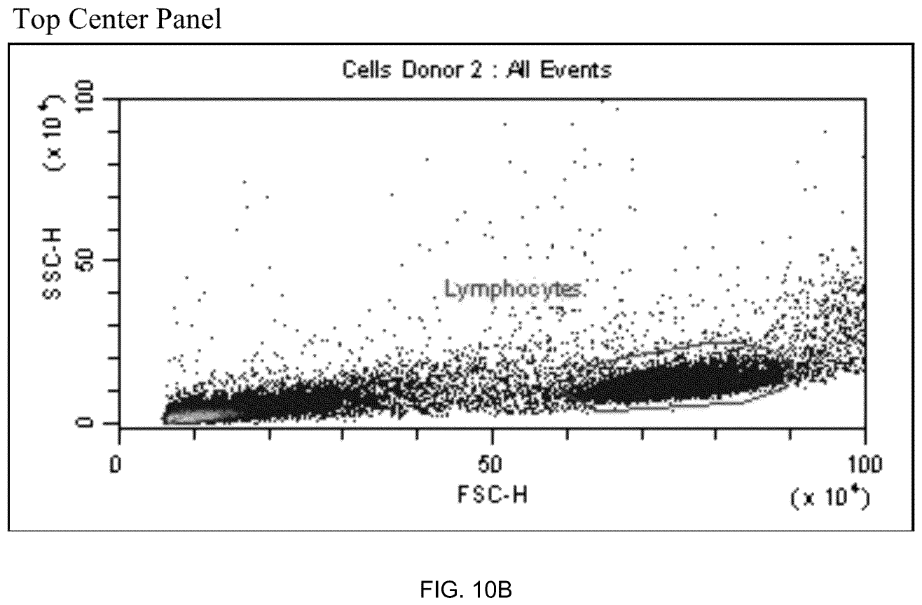

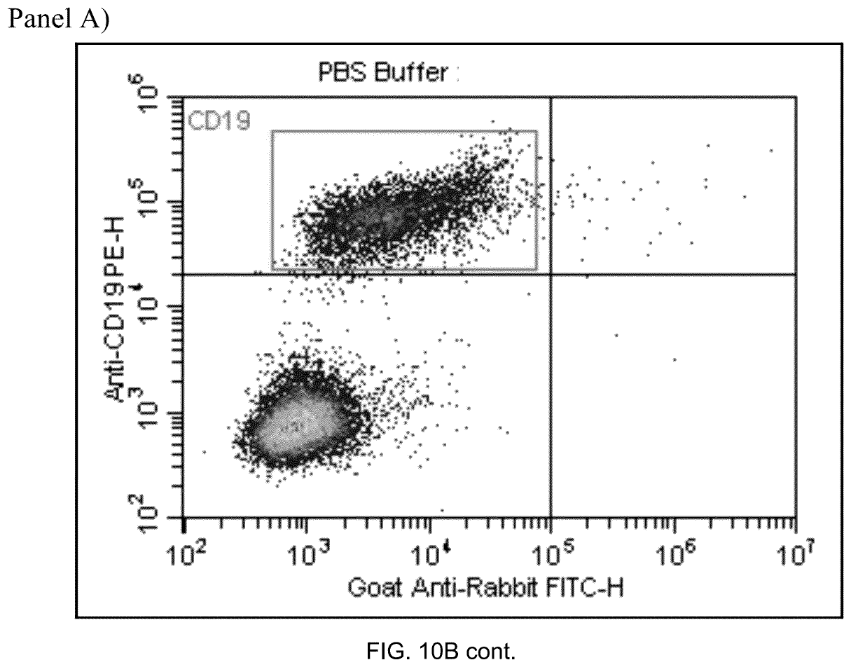

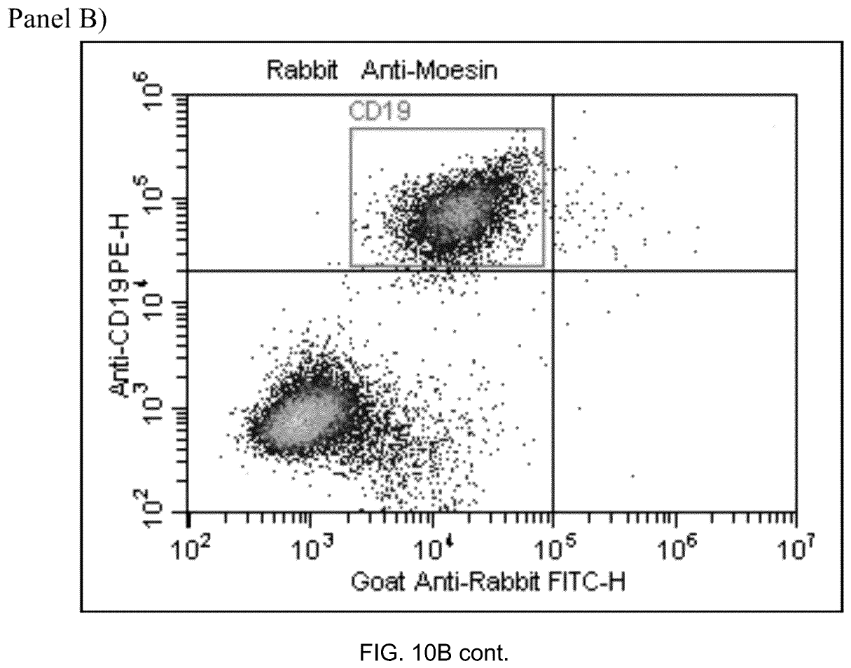

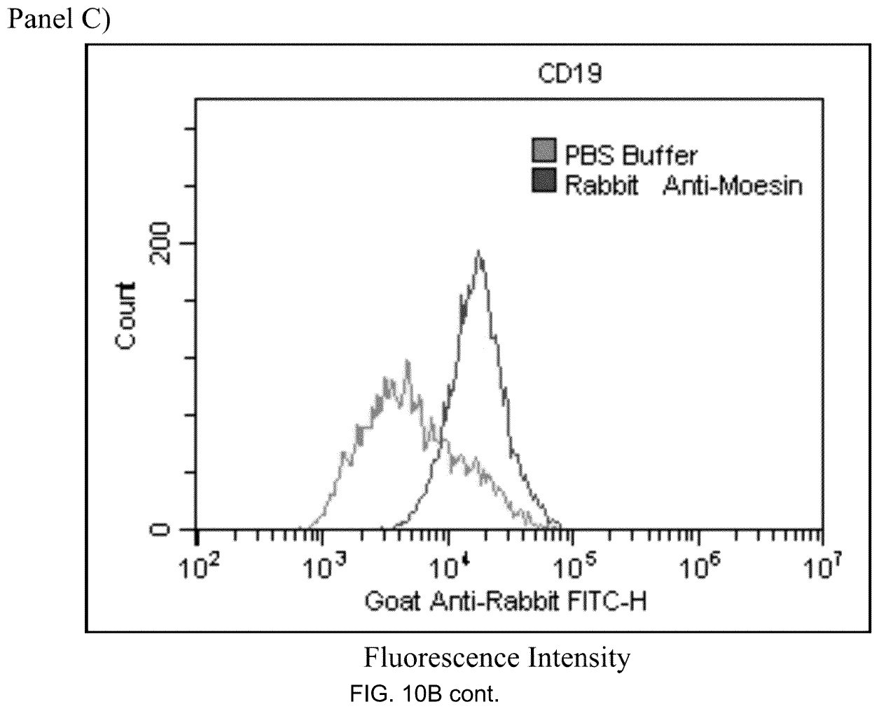

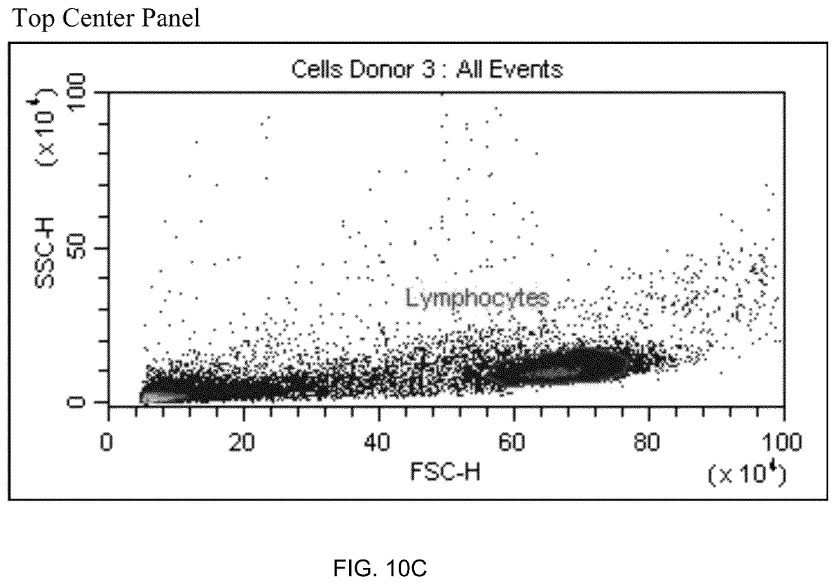

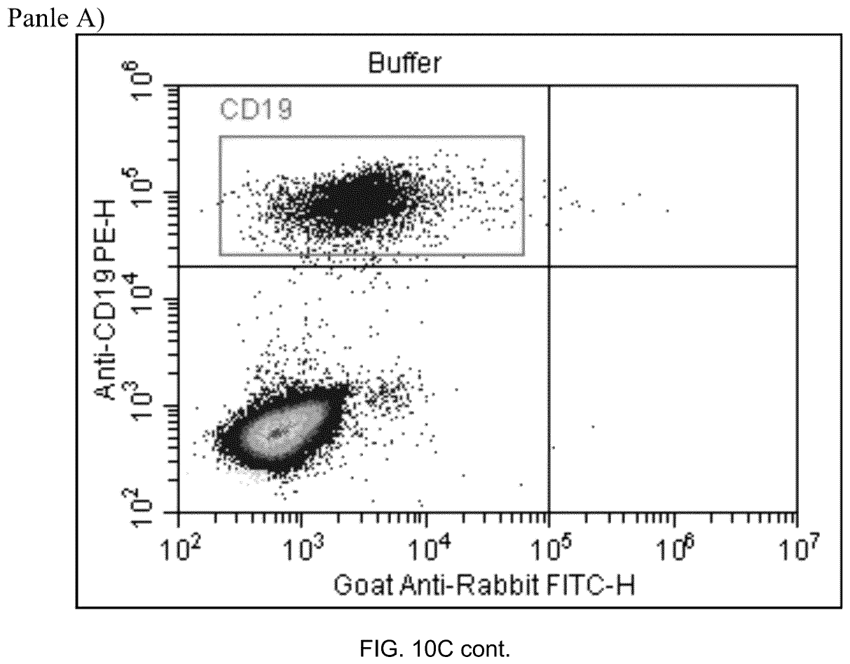

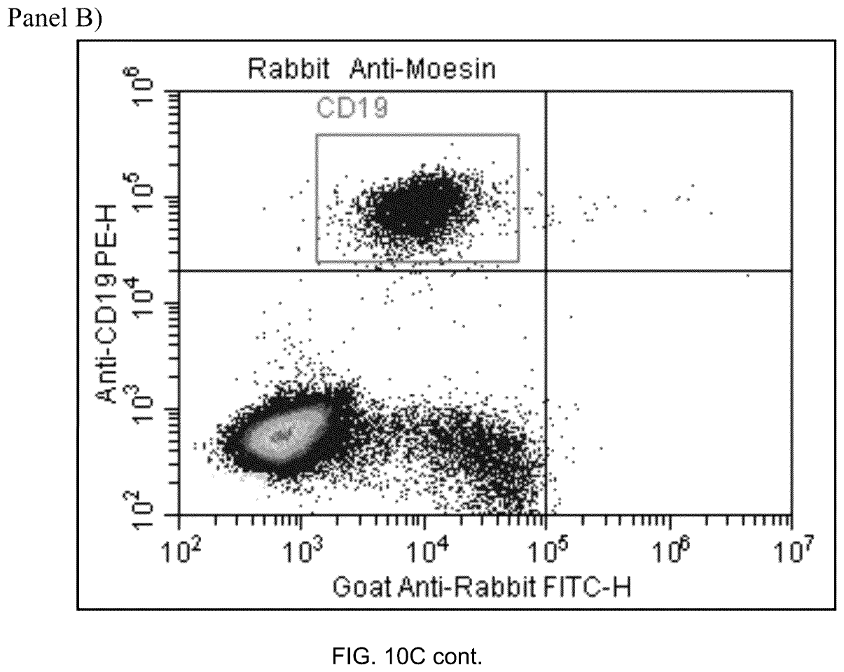

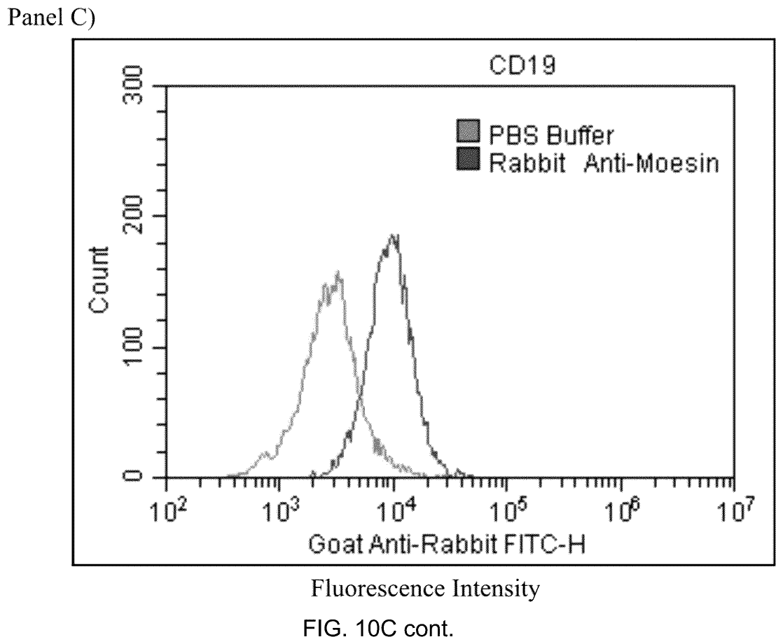

In flow cytometry assays of three healthy subjects (FIGS. 6A, 6B, 6C), moesin was detected on the exterior of the nNK cell plasma membrane with affinity purified anti-moesin antibody). Moesin was not detected on the surface of unstimulated T cells (FIGS. 6A, 6B and 6C, Panel C) and poorly expressed on B cells (FIG. 10A, 10B, 10C—Panel C). Cell surface expression of moesin on nNK cells was significantly greater than on T cells (94-fold, p=0.0087, FIG. 6D). All three cell types expressed the molecule intracellularly at similar levels in permeabilized lymphocytes (FIGS. 6E-6G).

More specifically, FIGS. 6A-6G depict reactivity with anti-moesin antibody indicates cell surface expression on nNK cells. Flow cytometry experiments were performed to determine if moesin was expressed on the surface of different lymphocyte subpopulations. Affinity purified rabbit anti-moesin antibody against amino acids 1 to 294 was used (0.3 μg) as described in Materials and Methods. Experiments comparing CD3⁺ cells with CD16⁺ lymphocytes from three subjects are shown in FIG. 6A Donor 1, FIG. 6B Donor 2, and FIG. 6C Donor 3, respectively. The top center panel of each figure shows the lymphocyte gate that was analyzed in subsequent panels. Freshly isolated peripheral blood lymphocytes were incubated with buffer as a negative control (Panel A, T cells and Panel D, NK cells of each donor) or treated with anti-moesin antibody (Panel B, T cells and Panel E, NK cells of each donor). T cells from Panel B of each donor were gated and further analyzed in Panel C. NK cells from Panel E of each donor were gated and further analyzed in Panel F. Non-permeabilized T cells were not reactive with the anti-moesin antibody (Panel C) whereas the non-permeabilized NK cells were highly reactive (Panel F). FIG. 6D shows the MFI values of the gated anti-moesin antibody treated CD3⁺ cells, treated CD16⁺ lymphocytes and CD19⁺ B cells. The average MFI of donor T cells (n=3) after subtraction of the buffer control MFI was 1,358±SD 1,200. The average MFI of donor NK cells (n=3) after buffer MFI subtraction was 128,119±SD 56,128. The mean MFI of donor B cells (n=3) after the buffer control MFI was subtracted was 11,814±SD 3.025. The null hypothesis: NK cells bind the anti-moesin antibody at a significantly higher level than T cells or B cells due to the presence of this protein on the cell surface (significant at p≤0.05). Student's unpaired t-test (one-tailed), df=4, NK cells vs T cells, p=0.0087, Student's unpaired t-test (one-tailed), df=4, NK cells vs B cells, p=0.0115, Student's unpaired t-test (one-tailed), df=4, T cells vs B cells, p=0.0026. Non-permeabilized CD19⁺ B cell flow cytometry experiments are found in FIGS. 10A-10C. Permeabilized NK cells (FIG. 6E), T cells (FIG. 6F), and B cells (FIG. 6G) all reacted strongly with anti-moesin antibody showing equivalent intracellular moesin expression.

More specifically, FIGS. 10A-10C depict expression of moesin on the surface of CD19⁺ B cells. Flow cytometry experiments were performed to determine if moesin was expressed on the surface of CD19⁺ B cells of three healthy subjects (See e.g., FIGS. 10A, 10B, 10C). Affinity purified rabbit anti-moesin antibody against amino acids 1 to 294 was used (0.3 μg) as described in Materials and Methods. The top center panel of each figure shows the lymphocyte gate that was subsequently analyzed. Panel A for each donor, gated buffer negative control, Panel B for each donor, gated anti-moesin antibody treated freshly isolated B cells, Panel C for each donor, gated negative control and gated antibody reactive cells from Panel A and B, respectively. The anti-moesin antibody reacted weakly with non-permeabilized CD19⁺ B cells.

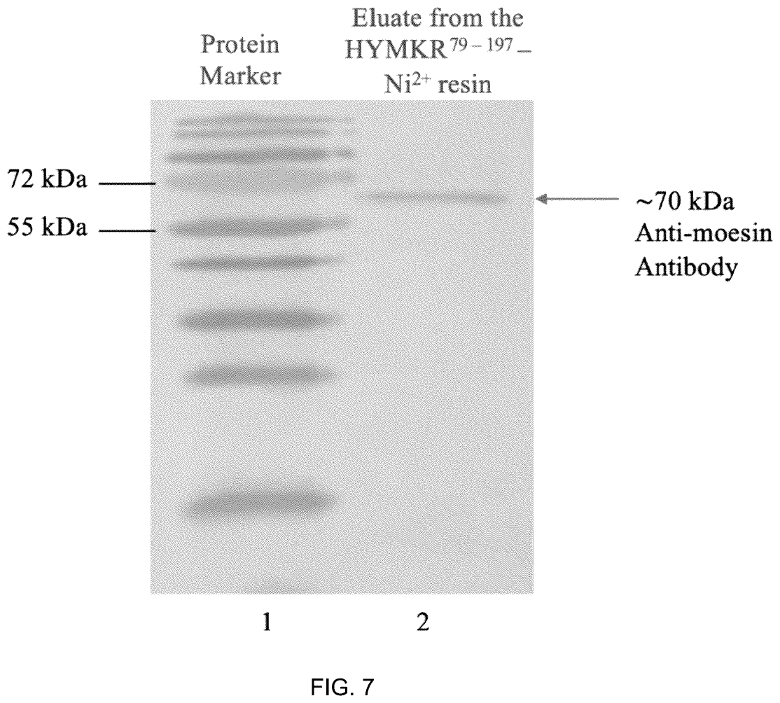

Additional experiments were performed to confirm the immunologic identity of the nNK cell plasma membrane protein that binds to the HYMKR⁷⁹⁻¹⁹⁷-Ni²⁺ resin. Western blots of the protein captured by affinity chromatography with the Ni²⁺ resin were probed with the affinity purified anti-moesin antibody. A single band was detected at approximately 70 kDa, indicating that the molecule captured by the HYMKR⁷⁹⁻¹⁹⁷-Ni²⁺ resin was indeed moesin (FIG. 7). The molecular weight of this band is similar to the surface biotinylated nNK cell protein that was captured by the HYMKR⁷⁹⁻¹⁹⁷-Ni²⁺ resin and probed with streptavidin-alkaline phosphatase (See FIG. 3B).

More specifically, FIG. 7 depicts immunological identity of the 70 kDa lymphocyte protein purified by affinity chromatography on a HYMKR⁷⁹⁻¹⁹⁷-Ni-resin. An extract of lymphocytes as a source of native moesin was applied to the HYMKR⁷⁹⁻¹⁹⁷-Ni²⁺ resin. The specifically bound protein(s) were eluted in a urea-imidazole buffer. Western blots were probed with a 1:1000 dilution of affinity purified anti-moesin antibody and then reacted with goat anti-rabbit IgG-alkaline phosphatase (1:1000). A single band of approximately 70 kDa was observed that reacted with the anti moesin antibody indicating that the protein purified by affinity chromatography on the HYMKR⁷⁹⁻¹⁹⁷-Ni²⁺ resin was moesin.

It has been demonstrated that HYMKR⁷⁹⁻¹⁹⁷ binds to the surface of nNK cells, and when bound to a solid support, pulls down a protein that contains moesin specific amino acid sequences (compared to ezrin and radixin). It is further show that anti-moesin antibody binds to the surface of nNK cells but not to T cells and binds to the 70 kDa protein isolated by affinity to the HYMKR⁷⁹⁻¹⁹⁷-Ni²⁺ resin. Overall, the above experiments demonstrate that moesin is the nNK cell surface receptor for HYMKR positive leukemia cell lines such as K562, Jurkat and Molt4, where their interaction initiates cytolysis.

### Discussion

Moesin, a member of a family of proteins that links the inner leaf of the plasma membrane to the cytoskeleton (See e.g., Fehon, R. G., A. I. McClatchey, and A. Bretscher, Organizing the cell cortex: the role of ERM proteins. Nat Rev Mol Cell Biol, 2010. 11(4): p. 276-87), is the nNK cell receptor for HYMKR/TOMM40. Only leukemia cell lines such as K562, Jurkat and Molt4 that express HYMKR/TOMM40 on their plasma membrane are susceptible to nNK cell mediated lysis by this pathway (See e.g., Das, B., et al., Preferential interaction of a novel tumor surface protein (p38.5) with naive natural killer cells. J Exp Med, 1997. 185(10): p. 1735-42; Das, B., R. Mushnitsky, and A. J. Norin, Difference in target cell recognition of naive and activated human natural killer cells: role of Haymaker (p38.5) in tumoricidal activity. Hum Immunol, 2005. 66(3): p. 241-51; and Das, B., et al., Genetic identity and differential expression of p38.5 (Haymaker) in human malignant and nonmalignant cells. Int J Cancer, 2001. 94(6): p. 800-6). Importantly, cell lines that lost expression of surface HYMKR were not susceptible to lysis by nNK cells. Cytolysis of K562 cells was blocked by pre-incubation of lymphocytes with soluble native HYMKR or recombinant HYMKR and was also blocked by pre-incubation of K562 cells with affinity purified anti-HYMKR antibodies directed against a peptide within HYMKR⁷⁹⁻¹⁹⁷ (See e.g., Das, B., et al., Preferential interaction of a novel tumor surface protein (p38.5) with naive natural killer cells. J Exp Med, 1997. 185(10): p. 1735-42; and Das, B., R. Mushnitsky, and A. J. Norin, Difference in target cell recognition of naive and activated human natural killer cells: role of Haymaker (p38.5) in tumoricidal activity. Hum Immunol, 2005. 66(3): p. 241-51).

It is now clear from the current experiments that the previously published blocking studies indicate that cell surface HYKMR was prevented from interacting with moesin on the surface of nNK cells. Though other receptor-ligand systems may be responsible for a proportion of leukemia cell line death, the interaction of moesin-HYMKR appears to be responsible for most of the killing of K562, Molt4 and Jurkat cells by nNK cells. It is noted that major death pathways have been described for NK cells involving the NCRs, p30, p44, p46 and NKG2D (See e.g., Vitale, M., et al., NKp44, a novel triggering surface molecule specifically expressed by activated natural killer cells, is involved in non-major histocompatibility complex-restricted tumor cell lysis. J Exp Med, 1998. 187(12): p. 2065-72; Pende, D., et al., Identification and molecular characterization of NKp30, a novel triggering receptor involved in natural cytotoxicity mediated by human natural killer cells. J Exp Med, 1999. 190(10): p. 1505-16; and Pende, D., et al., Role of NKG2D in tumor cell lysis mediated by human NK cells: cooperation with natural cytotoxicity receptors and capability of recognizing tumors of nonepithelial origin. Eur J Immunol, 2001. 31(4): p. 1076-86). However, these receptors appear to be restricted to activated NK cells (e.g., with Interleukin-2, IL-2) whereas the Moesin-HYMKR pathway is restricted to non-activated, nNK cells. It was previously shown that the cytolytic activity of IL-2 activated NK cells is distinct from nNK cells in that soluble recombinant HYMKR inhibited K562 lysis by nNK but did not block cytolysis of K562 by IL-2 activated NK cells (See e.g., Das, B., R. Mushnitsky, and A. J. Norin, Difference in target cell recognition of naive and activated human natural killer cells: role of Haymaker (p38.5) in tumoricidal activity. Hum Immunol, 2005. 66(3): p. 241-51).

Moesin is a member of the ERM family of proteins (ezrin, radixin, moesin). These molecules anchor the actin cytoskeleton to the inner leaf of the plasma membrane (See e.g., Fehon, R. G., A. I. McClatchey, and A. Bretscher, Organizing the cell cortex: the role of ERM proteins. Nat Rev Mol Cell Biol, 2010. 11(4): p. 276-87). ERM proteins play an important role in regulating various cellular activities, including membrane dynamics, cell migration, adhesion, survival, and reorganization of the actin cytoskeleton (See e.g., Ponuwei, G. A., A glimpse of the ERM proteins. J Biomed Sci, 2016. 23: p. 35; and Bretscher, A., K. Edwards, and R. G. Fehon, ERM proteins and merlin: integrators at the cell cortex. Nat Rev Mol Cell Biol, 2002. 3(8): p. 586-99). A role for ERM proteins in immune responses has been suggested in previous studies. Interestingly, male patients with hemizygous mutations in the moesin gene exhibit primary immunodeficiency that is referred to as X-linked moesin-associated immunodeficiency (See e.g., Lagresle-Peyrou, C., et al., X-linked primary immunodeficiency associated with hemizygous mutations in the moesin (MSN) gene. J Allergy Clin Immunol, 2016. 138(6): p. 1681-1689 e8).

A moesin-like receptor that presumably regulates IL-2 activated T cells by binding to an adhesion-modulating IL-2 peptide has been reported (See e.g., Ariel, A., et al., Cell surface-expressed moesin-like receptor regulates T cell interactions with tissue components and binds an adhesion-modulating IL-2 peptide generated by elastase. J Immunol, 2001. 166(5): p. 3052-60). In other reports, it was suggested that moesin functions as an independent LPS receptor on human monocytes and plays a role in lipopolysaccharide-induced TNF-α production (See e.g, Tohme, Z. N., S. Amar, and T. E. Van Dyke, Moesin functions as a lipopolysaccharide receptor on human monocytes. Infect Immun, 1999. 67(7): p. 3215-20; and Iontcheva, I., et al., Role for moesin in lipopolysaccharide-stimulated signal transduction. Infect Immun, 2004. 72(4): p. 2312-20).

Moesin and ezrin may function in activated T lymphocytes (See e.g., Shaffer, M. H., et al., Ezrin and moesin function together to promote T cell activation. J Immunol, 2009. 182(2): p. 1021-32). However, unlike NK cells, moesin is not expressed on the surface of quiescent T cells (this report). In the current study, we also show that moesin performs a novel function in nNK cells, namely as a receptor for cell-mediated cytotoxicity when it engages its ligand, HYMKR on leukemia cells.

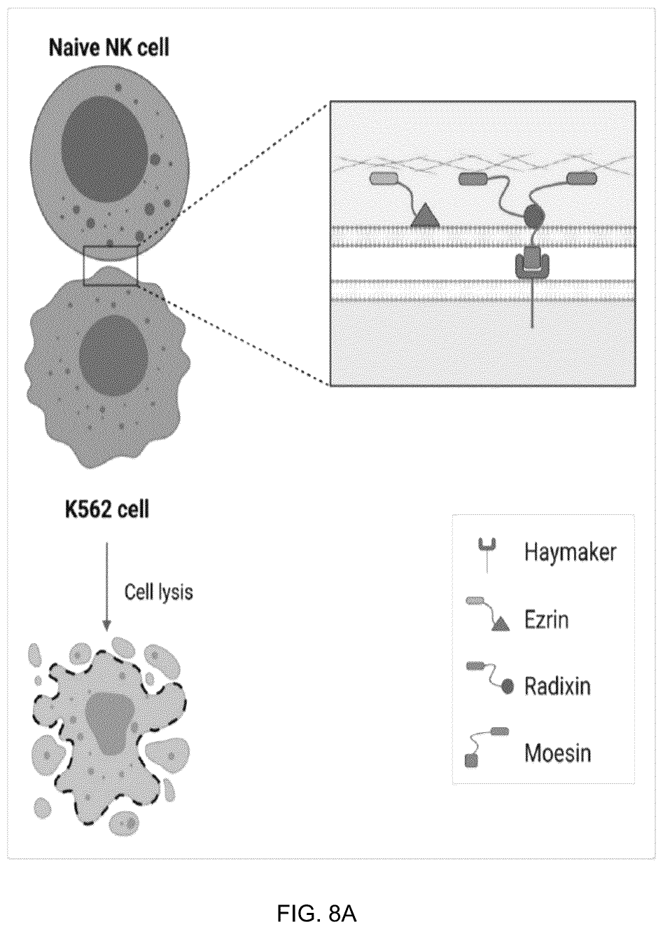

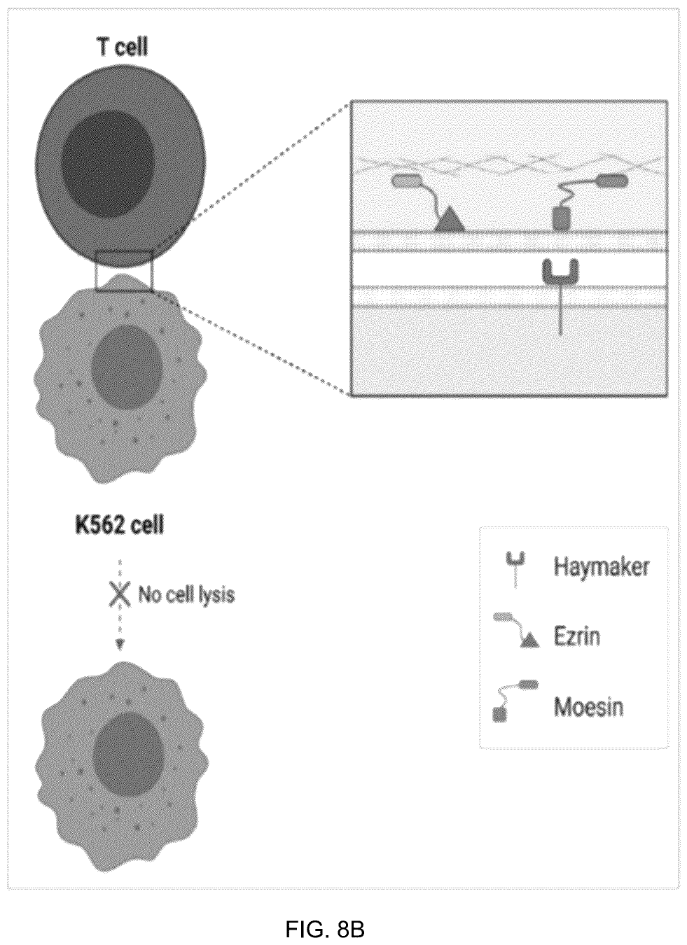

Interestingly, radixin is expressed in nNK and activated NK cells, but it is not expressed in resting or activated T cells (See e.g., Ramoni, C., et al., Differential expression and distribution of ezrin, radixin and moesin in human natural killer cells. Eur J Immunol, 2002. 32(11): p. 3059-65). The expression of radixin in nNK cells may allow for localization of moesin to the exterior of the plasma membrane, thereby permitting its interaction with HYMKR positive target cells as illustrated in FIGS. 8A and 8B.

More specifically, FIGS. 8A and 8B depict a model of surface localization of moesin in NK cells: interaction with radixin in nNK cells. (Panel A) Radixin, which is expressed in nNK cells and not in T cells, may interact with moesin thereby enabling localization of moesin to the cell surface, where it would interact with HYMKR on K562 target cells. (Panel B) The absence of radixin in T cells fails to promote surface localization of moesin.

Previously, incubation of K562 cells with an antibody against a 13-mer peptide, HYMKR¹⁸³⁻¹⁹⁵ inhibited nNK cell cytotoxicity (See e.g., Das, B., R. Mushnitsky, and A. J. Norin, Difference in target cell recognition of naive and activated human natural killer cells: role of Haymaker (p38.5) in tumoricidal activity. Hum Immunol, 2005. 66(3): p. 241-51). In contrast, the anti-13-mer antibody did not inhibit the cytolytic function of IL-2 activated NK cells (See e.g., Das, B., R. Mushnitsky, and A. J. Norin, Difference in target cell recognition of naive and activated human natural killer cells: role of Haymaker (p38.5) in tumoricidal activity. Hum Immunol, 2005. 66(3): p. 241-51). Another mitochondrial protein, the F1 β-subunit of H⁺ transporting ATP synthase, is expressed on the surface of epithelial-derived and hematopoietic-derived cancers (See e.g., Das, B., et al., A novel ligand in lymphocyte-mediated cytotoxicity: expression of the beta subunit of H+ transporting ATP synthase on the surface of tumor cell lines. J Exp Med, 1994. 180(1): p. 273-81; Moser, T. L., et al., Angiostatin binds ATP synthase on the surface of human endothelial cells. Proc Natl Acad Sci USA, 1999. 96(6): p. 2811-6; and Chang, H. Y., et al., Combination therapy targeting ectopic ATP synthase and 26S proteasome induces ER stress in breast cancer cells. Cell Death Dis, 2014. 5: p. e1540). An antibody against this molecule inhibited both nNK and IL-2 activated NK cell cytolytic activity (See e.g., Das, B., et al., A novel ligand in lymphocyte-mediated cytotoxicity: expression of the beta subunit of H+ transporting ATP synthase on the surface of tumor cell lines. J Exp Med, 1994. 180(1): p. 273-81). The F1 β-catalytic subunit of H⁺ transporting ATP synthase, may provide a source of energy at the cell surface for pumping ions across the plasma membrane, thereby inducing osmotic shock (also known as necrosis), leading to target cell lysis. Inhibition of cytolytic T cell activity by this antibody has not as yet been investigated.

Ostergaard and Clark described three death pathways used by cytolytic T lymphocytes. They include granule exocytosis-perforin/granzyme, Fas/FasL, and a Ca²⁺ dependent third pathway that does not utilize granule exocytosis or the Fas/FasL pathway (See e.g., Ostergaard, H. L. and W. R. Clark, Evidence for multiple lytic pathways used bycytotoxic Tlymphocytes. J Immunol, 1989. 143(7): p. 2120-6). More recently, Balint, S. et al. described a possible fourth pathway used by cytolytic T cells that involves autonomous supramolecular attack particles (SMAPs), which contain perforin and granzymes among many other proteins (See e.g., Balint, S., et al., Supramolecular attack particles are autonomous killing entities released from cytotoxic T cells. Science, 2020. 368(6493): p. 897-901). We propose that nNK cells use the third pathway that is initiated by the binding of Moesin to HYKMR and involves the surface activity of the F1 β-catalytic subunit of H⁺ transporting ATP synthase and Ca⁺² ions. Clearly, the immune system uses a number of different pathways to destroy abnormal cells. In this report, we identify a novel pathway initiated by the interaction of moesin on surface of nNK cells with HYMKR/TOMM40 surface positive target cells. The discovery of moesin as an nNK cell receptor in the cytolytic process may lead to a better understanding of the role of ERM proteins in innate immunity and provide new diagnostic and therapeutic opportunities. Furthermore, identification of moesin as an nNK cell receptor for HYMKR/TOMM40 will allow comprehensive investigations of this novel signaling pathway that leads to the destruction of leukemia cells.

All references are herein entirely incorporated by reference.

What is claimed is:

1. A polypeptide which is a variant of SEQ ID NO:1, fragment thereof, or fusion polypeptide, comprising: a variant of SEQ ID NO:1, or a fragment thereof, having an increased water solubility compared to SEQ ID NO:1 which binds to moesin, wherein the polypeptide comprises an affinity tag.

2. The polypeptide of claim 1, wherein the affinity tag is a polyhistidine tag or a hexahistidine tag.

3. The polypeptide of claim 1, wherein the affinity tag is bound to an amino or carboxy terminal end of the polypeptide.

4. The polypeptide of claim 1, wherein polypeptide comprises a plurality of deletions of amino acid residues corresponding to amino acid residues M1 to P78, 0198 to G361, or M1 to P78 and 0198 to G361 using SEQ ID NO:1 for numbering.

5. The polypeptide of claim 1, wherein the polypeptide comprises biotin.

6. The polypeptide of claim 1, wherein a sequence identity of the polypeptide to SEQ ID NO: 1 or SEQ ID NO: 2 is more than 90%, more than 95%, more than 96%, more than 97%, more than 98%, or more than 99%.

7. A variant of a parent HYMKR polypeptide comprising: a plurality of deletions of amino acid residues corresponding to amino acid residues M1 to P78 and 0198 to G361 using SEQ ID NO:1 for numbering, wherein the variant comprises biotin and increased water-solubility compared to the parent HYMKR polypeptide.

8. The variant of claim 7, further comprising an affinity tag.

9. The variant of claim 8, wherein the affinity tag comprises one or more positive electrically charged side chains comprising arginine, histidine, lysine, or combinations thereof.

10. The variant of claim 8, wherein the affinity tag is a polyhistidine tag or a hexahistidine tag.

11. The variant of claim 7, wherein the variant further comprises one or more additions of histidine amino acid residues corresponding to amino acid residues D198, D198 to F199, D198 to T200, D198 to A201, D198 to A202, or D198 to V203, using SEQ ID NO:1 for numbering, and wherein the variant has moesin protein binding activity and increased water solubility compared to the parent HYMKR polypeptide.

12. The variant of claim 7, wherein the parent HYMKR polypeptide is from Homo sapiens.

13. The variant of claim 7, wherein the parent HYMKR polypeptide comprises an amino acid sequence consisting of SEQ ID NO: 1.

14. The variant of claim 7, wherein the variant binds to moesin.

15. A method for preparing a polypeptide, fragment, or fusion polypeptide or fragment thereof, comprising:
a) contacting a polypeptide, fragment, variant, or fusion polypeptide precipitate obtained from bacteria cells with an aqueous urea solution in an amount sufficient to solubilize the precipitate and form a dissolved polypeptide, fragment, variant, or fusion polypeptide within an aqueous medium;
b) contacting the dissolved polypeptide, fragment, variant, or fusion polypeptide within the aqueous medium with biotin under conditions sufficient to bind biotin to the dissolved polypeptide, fragment, variant, or fusion polypeptide and form a biotinylated dissolved polypeptide, fragment, variant, or fusion polypeptide; and
c) recovering the biotinylated dissolved polypeptide, fragment, variant, or fusion polypeptide as a water-soluble biotinylated polypeptide, fragment, variant, or fusion polypeptide.

16. The method of claim 15, further comprising: purifying the water-soluble biotinylated polypeptide, fragment, variant, or fusion polypeptide.

17. The method of claim 15, further comprising, prior to (a): providing a nucleic acid sequence encoding a parent polypeptide; modifying the nucleic acid sequence to form a modified nucleic acid sequence encoding a polypeptide, fragment, variant, or fusion polypeptide; introducing the modified nucleic acid sequence into a suitable bacterial host cell; growing the bacterial host cells in a suitable growth medium under conditions leading to expression of the polypeptide, fragment, variant, or fusion polypeptide; and lysing the bacterial host cells to form a polypeptide, fragment, variant, or fusion polypeptide precipitate.

18. The method of claim 15, wherein a water-soluble biotinylated polypeptide, fragment, variant, or fusion polypeptide is a moesin binding partner.


SEQUENCE LISTING (20 sequences)

SEQ ID NO: 1 - protein, 361 aa, Homo sapiens
MGNVLAASSPPAGPPPPPAPALVGLPPPPPSPPGFTLPPLGGSLGAGTSTSRSSERTPGAATASASGAAEDGACGCLPNPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSDFTAAVTLGNPDVLVGSGILVAHYLQSITPCLALGGELVYHRRPGEEGTVMSLAGKYTLNNWLATVTLGQAGMHATYYHKASDQLQVGVEFEASTRMQDTSVSFGYQLDLPKANLLFKGSVDSNWIVGATLEKKLPPLPLTLALGAFLNHRKNKFQCGFGLTIG

SEQ ID NO: 2 - protein, 119 aa, Homo sapiens
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGS

SEQ ID NO: 3 - protein, 120 aa, Artificial Sequence
  Synthetic sequence
  MISC_FEATURE: X is one of R, H, K
  MISC_FEATURE: Xaa ia Arg, His, or Lys
  misc_feature (120)..(120): Xaa can be any naturally occurring amino acid
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSX

SEQ ID NO: 4 - protein, 121 aa, Artificial Sequence
  Synthetic sequence
  MISC_FEATURE: X is one or more of R, H, K
  MISC_FEATURE: Xaa ia Arg, His, or Lys
  misc_feature (120)..(121): Xaa can be any naturally occurring amino acid
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSXX

SEQ ID NO: 5 - protein, 122 aa, artificial sequence
  Synthetic sequence
  MISC_FEATURE: X is one or more of R, H, K
  MISC_FEATURE: Xaa ia Arg, His, or Lys
  misc_feature (120)..(122): Xaa can be any naturally occurring amino acid
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSXXX

SEQ ID NO: 6 - protein, 123 aa, artificial sequence
  Synthetic sequence
  MISC_FEATURE: X is one or more of R, H, K
  MISC_FEATURE: Xaa ia Arg, His, or Lys
  MISC_FEATURE: Xaa is one of Arg, His, Lys
  MISC_FEATURE: Xaa is one of Arg, Lys, His
  misc_feature (120)..(123): Xaa can be any naturally occurring amino acid
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSXXXX

SEQ ID NO: 7 - protein, 124 aa, Artificial Sequence
  Synthetic sequence
  MISC_FEATURE: X is one or more of R, H, K
  MISC_FEATURE: Xaa ia Arg, His, or Lys
  misc_feature (120)..(124): Xaa can be any naturally occurring amino acid
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSXXXXX

SEQ ID NO: 8 - protein, 125 aa, Artificial Sequence
  Synthetic sequence
  MISC_FEATURE: X is one or more of R, H, K
  misc_feature (120)..(125): Xaa can be any naturally occurring amino acid
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSXXXXXX

SEQ ID NO: 9 - protein, 120 aa, Artificial Sequence
  Synthetic sequence
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSH

SEQ ID NO: 10 - protein, 121 aa, Artificial Sequence
  Synthetic sequence
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSHH

SEQ ID NO: 11 - protein, 122 aa, Artificial Sequence
  Synthetic sequence
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSHHH

SEQ ID NO: 12 - protein, 123 aa, Artificial Sequence
  Synthetic sequence
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSHHHH

SEQ ID NO: 13 - protein, 124 aa, Artificial Sequence
  Synthetic sequence
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSHHHHH

SEQ ID NO: 14 - protein, 125 aa, Artificial Sequence
  Synthetic sequence
NPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSHHHHHH

SEQ ID NO: 15 - protein, 577 aa, Homo sapiens
MPKTISVRVTTMDAELEFAIQPNTTGKQLFDQVVKTIGLREVWFFGLQYQDTKGFSTWLKLNKKVTAQDVRKESPLLFKFRAKFYPEDVSEELIQDITQRLFFLQVKEGILNDDIYCPPETAVLLASYAVQSKYGDFNKEVHKSGYLAGDKLLPQRVLEQHKLNKDQWEERIQVWHEEHRGMLREDAVLEYLKIAQDLEMYGVNYFSIKNKKGSELWLGVDALGLNIYEQNDRLTPKIGFPWSEIRNISFNDKKFVIKPIDKKAPDFVFYAPRLRINKRILALCMGNHELYMRRRKPDTIEVQQMKAQAREEKHQKQMERAMLENEKKKREMAEKEKEKIEREKEELMERLKQIEEQTKKAQQELEEQTRRALELEQERKRAQSEAEKLAKERQEAEEAKEALLQASRDQKKTQEQLALEMAELTARISQLEMARQKKESEAVEWQQKAQMVQEDLEKTRAELKTAMSTPHVAEPAENEQDEQDENGAEASADLRADAMAKDRSEEERTTEAEKNERVQKHLKALTSELANARDESKKTANDMIHAENMRLGRDKYKTLRQIRQGNTKQRIDEFESM

SEQ ID NO: 16 - DNA, 44 bp, Artificial Sequence
  Synthetic sequence
ccataaccatgggtaacccgggcacattcgaggagtgccaccgt

SEQ ID NO: 17 - DNA, 36 bp, Artificial Sequence
  Synthetic sequence
ccatatctcgagagagccacgatactccccgtccac

SEQ ID NO: 18 - DNA, 357 bp, Artificial Sequence
  Synthetic Sequence
aacccgggcacattcgaggagtgccaccgtaagtgcaaggagctgtttcccattcagatggagggtgtcaagctcacagtcaacaaagggttgagtaaccattttcaggtcaaccacacagtagccctcagcacaatcggggagtccaactaccacttcggggtcacatatgtggggacaaagcagctgagtcccacagaggcgttccctgtactggtgggtgacatggacaacagtggcagtctcaacgctcaggtcattcaccagctgggccccggtctcaggtccaagatggccatccagacccagcagtcgaagtttgtgaactggcaggtggacggggagtatcgtggctct

SEQ ID NO: 19 - DNA, 357 bp, Artificial Sequence
  Synthetic sequence
aacccgggcacattcgaggagtgccaccggaagtgcaaggagctgtttcccattcagatggagggtgtcaagctcacagtcaacaaagggttgagtaaccattttcaggtcaaccacacagtagccctcagcacaatcggggagtccaactaccacttcggggtcacatatgtggggacaaagcagctgagtcccacagaggcgttccctgtactggtgggtgacatggacaacagtggcagtctcaacgctcaggtcattcaccagctgggccccggtctcaggtccaagatggccatccagacccagcagtcgaagtttgtgaactggcaggtggacggggagtatcggggctct

SEQ ID NO: 20 - DNA, 1563 bp, Homo sapiens
gcaggcgaccatggggaacgtgttggctgccagctcgccgcccgcagggccgccaccgccgcctgcgccggccctcgtggggctgccgccacctccgccctcgccgccgggcttcacgctgccgccgctgggaggcagcctgggcgccggcaccagtacgagtcgaagttcggaacggacccccggggctgcaaccgccagcgcctcaggggccgccgaggatggggcctgcggctgcctgcccaacccgggcacattcgaggagtgccaccggaagtgcaaggagctgtttcccattcagatggagggtgtcaagctcacagtcaacaaagggttgagtaaccattttcaggtcaaccacacagtagccctcagcacaatcggggagtccaactaccacttcggggtcacatatgtggggacaaagcagctgagtcccacagaggcgttccctgtactggtgggtgacatggacaacagtggcagtctcaacgctcaggtcattcaccagctgggccccggtctcaggtccaagatggccatccagacccagcagtcgaagtttgtgaactggcaggtggacggggagtatcggggctctgacttcacagcagccgtcaccctggggaacccagacgtcctcgtgggttcaggaatcctcgtagcccactacctccagagcatcacgccttgcctggccctgggtggagagctggtctaccaccggcggcctggagaggagggcactgtcatgtctctagctgggaaatacacattgaacaactggttggcaacggtaacgttgggccaggcgggcatgcacgcaacatactaccacaaagccagtgaccagctgcaggtgggtgtggagtttgaggccagcacaaggatgcaggacaccagcgtctccttcgggtaccagctggacctgcccaaggccaacctcctcttcaaaggctctgtggatagcaactggatcgtgggtgccacgctggagaagaagctcccacccctgcccctgacactggcccttggggccttcctgaatcaccgcaagaacaagtttcagtgtggctttggcctcaccatcggctgagccctcctggcccccgccttccacgcccttccgattccacctccacctccacctccccctgccacagaggggagacctgagcccccctcccttccctccccccttgggggtcggggggggacattggaaaggagggaccccgccaccccagcagctgaggaggggattctggaactgaatggcgcttcgggattctgagtagcaggggcagcatgcccagtgggcctggggtcccgggagggattccggaattgaggggcacgcaggattctgagcaccaggggcagaggcggccagacaacctcagggaggagtgtcctggcgtccccatcctccaaagggcctgggcccgccccgagggggcagcgagaggagcttccccatccccggtcagtccaccctgccccgtccactttcccatctcctcggtataaatcatgtttataagttatggaagaaccgggaca